SEQUENCE LISTING (181 sequences)

SEQ ID NO: 1 - protein, 10 aa, Artificial Sequence
  Synthetic sequence
GFSLXXXGXH

SEQ ID NO: 2 - protein, 16 aa, Artificial Sequence
  Synthetic sequence
VIWXGGXTXXNAXFXS

SEQ ID NO: 3 - protein, 10 aa, Artificial Sequence
  Synthetic sequence
RASXSVXXXX

SEQ ID NO: 4 - protein, 11 aa, Artificial Sequence
  Synthetic sequence
XASQDXXXXLX

SEQ ID NO: 5 - protein, 7 aa, Artificial Sequence
  Synthetic sequence
RAXRLXD

SEQ ID NO: 6 - protein, 7 aa, Artificial Sequence
  Synthetic sequence
XXSXXXS

SEQ ID NO: 7 - protein, 9 aa, Artificial Sequence
  Synthetic sequence
XQXXXXPXT

SEQ ID NO: 8 - protein, 9 aa, Artificial Sequence
  Synthetic sequence
LXXDXFPXT

SEQ ID NO: 9 - protein, 5 aa, Artificial Sequence
  Synthetic sequence
GGGGS

SEQ ID NO: 10 - protein, 20 aa, Artificial Sequence
  Synthetic sequence
GGGGSGGGGSGGGGSGGGGS

SEQ ID NO: 11 - protein, 6 aa, Artificial Sequence
  Synthetic sequence
SGGGGS

SEQ ID NO: 12 - protein, 244 aa, Artificial Sequence
  Synthetic sequence
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 13 - protein, 12 aa, Artificial Sequence
  Synthetic sequence
GFSLXXXGXXXX

SEQ ID NO: 14 - protein, 11 aa, Artificial Sequence
  Synthetic sequence
GXXXXXXXXXX

SEQ ID NO: 15 - protein, 17 aa, Artificial Sequence
  Synthetic sequence
XXXXXXXXXXYXXXXXX

SEQ ID NO: 16 - protein, 4 aa, Artificial Sequence
  Synthetic sequence
XYYX

SEQ ID NO: 17 - protein, 7 aa, Artificial Sequence
  Synthetic sequence
XXSXXXS

SEQ ID NO: 18 - protein, 9 aa, Artificial Sequence
  Synthetic sequence
XQXXXXPXT

SEQ ID NO: 19 - protein, 9 aa, Artificial Sequence
  Synthetic sequence
LXXDXFPXT

SEQ ID NO: 20 - protein, 111 aa, Artificial Sequence
  Synthetic sequence
EIVLTQSPATLSLSPGERATLSCRASESVDNYGISFMHWYQQKPGQAPRLLIYRASNLESGIPARFSGSGSGTDFTLTISSLEPEDFAVYYCQQSNKDPYTFGQGTKVEIK

SEQ ID NO: 21 - protein, 111 aa, Artificial Sequence
  Synthetic sequence
DIVLTQSPATLSLSPGERATLSCRASESVDNYGISFMHWYQQKPGQAPRLLIYRASNLESGIPARFSGSGSGTDFTLTISSLEPEDFAVYYCQQSNKDPYTFGQGTKVEIK

SEQ ID NO: 22 - protein, 111 aa, Artificial Sequence
  Synthetic sequence
DIVLTQSPATLSLSPGERATISCRASESVDNYGISFMHWYQQKPGQAPRLLIYRASNLESGIPARFSGSGSGTDFTLTISSLEPEDFAVFYCQQSNKDPYTFGQGTKVEIK

SEQ ID NO: 23 - protein, 111 aa, Artificial Sequence
  Synthetic sequence
DIVLTQSPATLSLSPGERATISCRASESVDNYGISFMHWYQQKPGQAPRLLIYRASNLESGIPARFSGSGSRTDFTLTISSLEPEDFAVFYCQQSNKDPYTFGQGTKVEIK

SEQ ID NO: 24 - protein, 118 aa, Artificial Sequence
  Synthetic sequence
EVQLVESGGGLVQPGGSLRLSCAASGYSITSGYYWNWVRQAPGKGLEWISYISYDGSNNYNPSLKNRFTISRDSAKNSLYLHMHSLRAEDTAVYYCARDAYSYGMDYWGQGTTVTVSS

SEQ ID NO: 25 - protein, 118 aa, Artificial Sequence
  Synthetic sequence
EVQLVESGGGLVQPGGSLRLSCAVSGYSITSGYYWNWVRQAPGKGLEGISYISYDGSNNYNPSLKNRFTISRDSAKNSFYLHMHSLRAEDTAVYYCARDAYSYGMDYWGQGTTVTVSS

SEQ ID NO: 26 - protein, 118 aa, Artificial Sequence
  Synthetic sequence
EVQLVESGGGLVQPGGSLRLSCAVSGYSITSGYYWNWIRQAPGKGLEGIGYISYDGSNNYNPSLKNRITISRDSAKNSFYLHMHSLRAEDTAVYYCARDAYSYGMDYWGQGTTVTVSS

SEQ ID NO: 27 - protein, 118 aa, Artificial Sequence
  Synthetic sequence
DVQLVESGGGLVQPGGSLRLSCAVTGYSITSGYYWNWIRQAPGKGLEGIGYISYDGSNNYNPSLKNRITISRDSAKNSFYLHMHSLRAEDTAVYYCARDAYSYGMDYWGQGTTVTVSS

SEQ ID NO: 28 - protein, 118 aa, Artificial Sequence
  Synthetic sequence
DVQLVESGGGLVQPGGSLRLSCAVTGYSITSGYYWNWIRQAPGKGLEGMGYISYDGSNNYNPSLKNRITISRDSAKNSFYLHLHSLRAEDTAVYYCARDAYSYGMDYWGQGTTVTVSS

SEQ ID NO: 29 - protein, 218 aa, Artificial Sequence
  Synthetic sequence
EIVLTQSPATLSLSPGERATLSCRASESVDNYGISFMHWYQQKPGQAPRLLIYRASNLESGIPARFSGSGSGTDFTLTISSLEPEDFAVYYCQQSNKDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 447 aa, Artificial Sequence
  Synthetic sequence
EVQLVESGGGLVQPGGSLRLSCAVSGYSITSGYYWNWVRQAPGKGLEGISYISYDGSNNYNPSLKNRFTISRDSAKNSFYLHMHSLRAEDTAVYYCARDAYSYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 218 aa, Artificial Sequence
  Synthetic sequence
EIVLTQSPATLSLSPGERATLSCRASESVDNYGISFMHWYQQKPGQAPRLLIYKASNLESGIPARFSGSGSGTDFTLTISSLEPEDFAVYYCQQSNKDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 218 aa, Artificial Sequence
  Synthetic sequence
EIVLTQSPATLSLSPGERATLSCRASESVDNYGISFMHWYQQKPGQAPRLLIYRASSLESGIPARFSGSGSGTDFTLTISSLEPEDFAVYYCQQSNKDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 218 aa, Artificial Sequence
  Synthetic sequence
EIVLTQSPATLSLSPGERATLSCRASESVDNYGISFMHWYQQKPGQAPRLLIYKASSLESGIPARFSGSGSGTDFTLTISSLEPEDFAVYYCQQSNKDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 218 aa, Artificial Sequence
  Synthetic sequence
AIQLTQSPSSLSASVGDRVTITCRASESVDNYGISFMHWYQQKPGKAPKLLIYKASSLESGVPSRFSGSGSGTDFTLTISSLQPEDFATYYCQQSNKDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 218 aa, Artificial Sequence
  Synthetic sequence
DIQLTQSPSSLSASVGDRVTITCRASESVDNYGISFMHWYRQKPGKAPKLLIYKASSLESGVPSRFSGRGSGTDFTLTISSLQPEDFATYYCQQSNKDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 218 aa, Artificial Sequence
  Synthetic sequence
DIRLTQSPSSLSASVGQRVTISCRASESVDNYGISFMHWYRQKPGKAPKLLIYKASSLESGVPSRFSGRGSGTDFTLTISSLQPEDFATYYCQQSNKDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 447 aa, Artificial Sequence
  Synthetic sequence
EVQLVESGGGLVQPRGSLRLSCAVSGYSITSGYYWNWIRQAPGKGLEWVSYISYDGSNNYNPSLKNRFTISRDNSKNTFYLQMNNLRAEDTAAYYCARDAYSYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 447 aa, Artificial Sequence
  Synthetic sequence
EVQLQESGGGLVKPRGSLRLSCAVSGYSITSGYYWNWIRQAPGKGLEWVSYISYDGSNNYNPSLKNRFSISRDNSKNTFYLKMNRLRAEDSAAYYCARDAYSYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 112 aa, Artificial Sequence
  Synthetic sequence
DIVMTQSPLSLPVTPGEPASISCRSSQNIVHSNGNTYLEWYLQKPGQSPQLLIYKVSNRFSGVPDRFSGSGSGTDFTLKISRVEAEDVGVYYCFQGSHFPWTFGQGTKVEIK

SEQ ID NO: 40 - protein, 112 aa, Artificial Sequence
  Synthetic sequence
DVVMTQSPLSLPVTPGEPASISCRSSQNIVHSNGNTYLEWYLQKPGQSPQLLIYKVSNRFSGVPDRFSGSGSGTDFTLKISRVEAEDVGVYYCFQGSHFPWTFGQGTKVEIK

SEQ ID NO: 41 - protein, 118 aa, Artificial Sequence
  Synthetic sequence
QVQLVESGGGLVKPGGSLRLSCAASGFTFSDYYMYWIRQAPGKGLEWVSTIGDGTSYTHYPDSVQGRFTISRDNAKNSLYLQMNSLRAEDTAVYYCARDLGTGPFAYWGQGTLVTVSS

SEQ ID NO: 42 - protein, 118 aa, Artificial Sequence
  Synthetic sequence
QVQLVESGGGLVKPGGSLRLSCAVSGFTFSDYYMYWIRQAPGKGLEWVSTIGDGTSYTHYPDSVQGRFTISRDNAKNSLYLQMNSLRAEDTAVYYCARDLGTGPFAYWGQGTLVTVSS

SEQ ID NO: 43 - protein, 118 aa, Artificial Sequence
  Synthetic sequence
EVQLVESGGGLVKPGGSLRLSCAVSGFTFSDYYMYWIRQAPGKGLEWVSTIGDGTSYTHYPDSVQGRFTISRDYAKNSLYLQMNSLRAEDTAVYYCARDLGTGPFAYWGQGTLVTVSS

SEQ ID NO: 44 - protein, 118 aa, Artificial Sequence
  Synthetic sequence
EVKLVESGGGLVKPGGSLRLSCAVSGFTFSDYYMYWIRQAPGKGLEWVSTIGDGTSYTHYPDSVQGRFTISRDYAKNSLYLQMNSLRAEDTAVYYCARDLGTGPFAYWGQGTLVTVSS

SEQ ID NO: 45 - protein, 118 aa, Artificial Sequence
  Synthetic sequence
EVKLVESGGGLVKPGGSLRLSCAVSGFTFSDYYMYWIRQAPGKGLEWVSTIGDGTSYTHYPDSVQGRFTISRDYATNNLYLQMNSLRAEDTAVYYCARDLGTGPFAYWGQGTLVTVSS

SEQ ID NO: 46 - protein, 122 aa, Mouse
QVQLKQSGPGLVQPSQSLSITCTVSGFSLSTYGVHWVRQFPGKGLEWLGVIWRGGNTNYNAAFMSRLTISKDNSKSQVFFKMNSLQAKDTAIYYCVRNQIYDGYYDYAMDYWGQGTSVTVSS

SEQ ID NO: 47 - protein, 19 aa, Mouse
MAVLGLLFCLVTFPSCVLS

SEQ ID NO: 48 - DNA, 423 bp, Mouse
atggctgtcctggggctgctcttctgcctggtgacattcccaagctgtgtcctgtcccaggtgcagctgaagcagtcaggacctggcctagtgcagccctcacagagcctgtccatcacctgcacagtctctggtttctcattatctacctatggtgtccactgggttcgccagtttccaggaaagggtctggagtggctgggagtgatatggagaggtggaaacacaaactataatgcagctttcatgtccagactgaccatcagcaaggacaattccaagagtcaagttttctttaaaatgaacagtctgcaagctaaagacacagccatatattattgtgtcagaaaccagatctatgatggttactacgactatgctatggactactggggtcagggaacctcagtcaccgtctcctca

SEQ ID NO: 49 - protein, 107 aa, Mouse
DIKMTQSPSSMYASLGERVTITCKASQDINTYLNWLQQKPGKSPKTLIYRANRLVDGVPSRFSGRGSGQDYSLTISSLEYEDVGIYYCLHYDAFPWTFGGGTKLEIK

SEQ ID NO: 50 - protein, 20 aa, Mouse
MRAPAQFFGFLLLWFPGIKC

SEQ ID NO: 51 - DNA, 381 bp, Mouse
atgagggcccctgctcagttttttggcttcttgttgctctggtttccaggtatcaaatgtgacatcaagatgacccagtctccatcttccatgtatgcatctctaggagagagagtcactatcacttgcaaggcgagtcaggacattaatacctatttaaactggctccagcagaaaccagggaaatctcctaagaccctgatctatcgtgcaaacagattggtagatggggtcccatcaaggttcagtggccgtggatctgggcaagattattctctcaccatcagcagcctggaatatgaagatgtgggaatttattattgtctacactatgatgcatttccgtggacgttcggcggaggcaccaagctggaaatcaaa

SEQ ID NO: 52 - protein, 116 aa, Mouse
EVQLQQSGPELVKPGASVKISCKASGYSFTGYFMNWMRQSHGKSLEWIGRINPYNGDSFYNQKFKDKATLTVDKSSTTAHMELLSLTSEDSAVYYCGRGYDAMDYWGQGTSVTVSS

SEQ ID NO: 53 - protein, 19 aa, Mouse
MGWSCVMLFLLSVTVGVFS

SEQ ID NO: 54 - DNA, 405 bp, Mouse
atgggatggagctgtgtaatgctctttctcctgtcagtaactgtaggtgtgttttctgaggttcagctgcagcagtctggacctgagctggtgaagcctggggcttcagtgaagatatcctgcaaggcttctggttactcatttactggctactttatgaactggatgaggcagagccatggaaagagccttgagtggattggacgtattaatccttacaatggtgattctttctacaaccagaagttcaaggacaaggccacattgactgtagacaaatcctctaccacagcccacatggagctcctgagcctgacatctgaggactctgcagtctattattgtggaagaggatacgacgctatggactactggggtcaaggaacctcagtcaccgtctcctca

SEQ ID NO: 55 - protein, 107 aa, Mouse
DIQMTQTTSSLSASLGDRVTISCRASQDISNFLTWYQQKPDGTVKLLIYYTSKLHSGVPSRFSGSGSGTDYSLTISNLEPGDIATYYCQQVSKFPWTFGGGAKLEIK

SEQ ID NO: 56 - protein, 20 aa, Mouse
MVSTAQFLGLLLLCFQGTRC

SEQ ID NO: 57 - DNA, 381 bp, Mouse
atggtgtccacagctcagttccttggtctcctgttgctctgttttcaaggtaccagatgtgatatccagatgacacagactacatcctccctgtctgcctctctgggagacagagtcaccattagttgcagggcaagtcaggacattagcaattttttaacctggtatcagcagaaaccagatggaactgttaaactcctgatctactacacatcaaaattacactcaggagtcccatcaaggttcagtggcagtgggtctgggacagattattctctcaccattagcaacctggaaccgggtgatattgccacttactattgccaacaggttagtaagtttccgtggacgttcggtggaggcgccaagctggaaatcaaa

SEQ ID NO: 58 - protein, 114 aa, Mouse
QVQLQQSGPELVKPGASVQISCKASGYVFSSSWMNWVKQRPGRGLEWIGRIYPGDGDTDYTGKFKGKATLTADKSSNTAYMQLSSLTSVDSAVYFCASGYFDFWGQGTPLTVSS

SEQ ID NO: 59 - protein, 19 aa, Mouse
MGWSCIMFFLLSITAGVHC

SEQ ID NO: 60 - DNA, 399 bp, Mouse
atgggatggagctgtatcatgttcttcctcctgtcaataactgcaggtgtccattgccaggtccagctgcagcagtctggacctgagctggtgaagcctggggcctcagtgcagatttcctgcaaagcttctggctacgttttcagtagttcttggatgaactgggtgaagcagaggcctggacggggtcttgagtggattgggcggatttatcctggagatggagatactgactacactgggaagttcaagggcaaggccacactgactgcagacaaatcctccaacacagcctacatgcagctcagcagcctgacctctgtggactctgcggtctatttctgtgcaagtgggtactttgacttctggggccaaggcacccctctcaccgtctcctca

SEQ ID NO: 61 - protein, 107 aa, Mouse
DITMTQSPSSMYASLGERVTITCKASQDMNNYLRWFQQKPGKSPQTLIFRANRLVDGVPSRFSGSGSGQDYSLTISSLEFEDMGIYYCLQHDKFPPTFGGGTKLEIK

SEQ ID NO: 62 - protein, 20 aa, Mouse
MRAPAQFLGILLLWFPGIKC

SEQ ID NO: 63 - DNA, 381 bp, Mouse
atgagggcccctgctcagtttcttggcatcttgttgctctggtttccaggtatcaaatgtgacatcacgatgacccagtctccatcttccatgtatgcatctctaggagagagagtcactatcacttgcaaggcgagtcaggacatgaataactatttaaggtggttccagcagaaaccagggaagtctcctcagaccctgatctttcgtgcaaacagattggtcgatggggtcccatcaaggttcagtggcagtggatctgggcaagattattctctcaccatcagcagcctggaatttgaagatatgggaatttattattgtctacagcatgataaatttcctccgacgttcggtggaggcaccaagctggaaatcaaa

SEQ ID NO: 64 - protein, 118 aa, Mouse
EVKLVESGGGLVKPGGSLKLSCAVSGFTFSDYYMYWIRQTPEKRLEWVATIGDGTSYTHYPDSVQGRFTISRDYATNNLYLQMTSLRSEDTALYYCARDLGTGPFAYWGQGTLVTVSA

SEQ ID NO: 65 - protein, 19 aa, Mouse
MDFGLSWVFLVLVLKGVQC

SEQ ID NO: 66 - DNA, 411 bp, Mouse
atggacttcgggttgagctgggttttccttgtccttgttttaaaaggtgtccagtgtgaagtgaagctggtggagtctggaggaggcttagtgaagcctggagggtccctgaaactctcctgtgcagtctctggattcactttcagtgactattatatgtattggattcgccagactccggaaaagcggctggagtgggtcgcaaccattggtgatggtactagttacacccactatccagacagtgtgcaggggcgattcaccatctccagagactatgccacgaacaacctgtacctgcaaatgactagtctgaggtctgaagacacagccttatattactgtgcaagagatcttggaaccgggccttttgcttactggggccaggggactctggtcactgtctctgca

SEQ ID NO: 67 - protein, 112 aa, Mouse
DVLMTQTPRSLPVSLGDQASISCRSSQNIVHSNGNTYLEWYLQKPGQSPKLLIYKVSNRFSGVPDRFSGSGSGTDFTLKISRVEAEDLGVYYCFQGSHFPWTFGGGTKLEIK

SEQ ID NO: 68 - protein, 19 aa, Mouse
MKLPVRLLVLMFWIPASSS

SEQ ID NO: 69 - DNA, 393 bp, Mouse
atgaagttgcctgttaggctgttggtgctgatgttctggattcctgcttccagcagtgacgttttgatgacccaaactccacgctccctgcctgtcagtcttggagatcaagcctccatctcttgcagatctagtcagaacattgttcatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacattttccttggacattcggtggaggcaccaagctggagatcaaa

SEQ ID NO: 70 - protein, 118 aa, Mouse
DVQLQESGPGLVKPSQSLSLTCSVTGYSITSGYYWNWIRQFPGNKLEGMGYISYDGSNNYNPSLKNRISITRDSSKNQFFLKLNSVTAEDSGTYYCARDAYSYGMDYWGQGTSVTVSS

SEQ ID NO: 71 - protein, 18 aa, Mouse
MMVLSLLYLLTAIPGILS

SEQ ID NO: 72 - DNA, 411 bp, Mouse; identical to SEQ ID NO: 66

SEQ ID NO: 73 - protein, 111 aa, Mouse
DIVLTQSPASLAVSLGQRATISCRASESVDNYGISFMHWYQQKPGQPPKLLIYRASNLESGIPARFSGSGSRTDFTLTINPVETDDVATFYCQQSNKDPYTFGGGTKLEIK

SEQ ID NO: 74 - protein, 20 aa, Mouse
METDTLLLWVLLLWVPGSTG

SEQ ID NO: 75 - DNA, 393 bp, Mouse
atggagacagacacactcctgctatgggtgctgctgctctgggttccaggttccacaggtgacattgtgctgacccaatctccagcttctttggctgtgtctctagggcagagggccaccatctcctgcagagccagcgaaagtgttgataattatggcattagttttatgcactggtaccagcagaaaccaggacagccacccaaactcctcatctatcgtgcatccaacctagaatctgggatccctgccaggttcagtggcagtgggtctaggacagacttcaccctcaccattaatcctgtggagactgatgatgttgcaaccttttactgtcagcaaagtaataaggatccgtacacgttcggaggggggaccaagctggaaataaaa

SEQ ID NO: 76 - protein, 121 aa, Mouse
QVQLKQSGPGLVQPSQNLSITCTVSGFSLTNYGIHWIRQPPGKGLEWLGVIWSGGSTDHNAAFISRLSISKDNSKSQVFFTMNSLEVDDTAIYYCARNRAYYRYEGGMDYWGQGTSVTVSS

SEQ ID NO: 77 - protein, 19 aa, Mouse
MAVLGLLFCLVTFPSCVLS

SEQ ID NO: 78 - DNA, 420 bp, Mouse
atggctgtcctggggctgctcttctgcctggtgacattcccaagctgtgtcctatcccaggtgcagctgaaacagtcaggacctggcctcgtgcagccctcacagaacctgtccatcacctgcacagtctctggtttctcattaactaactatggtatacactggattcgccagcctccaggaaagggtctggagtggctgggagtgatatggagtggtggaagcacagaccataatgctgctttcatatccagactgagcatcagcaaggacaactccaagagccaagttttctttacaatgaacagtctggaagttgatgacacagccatatactactgtgccagaaatagagcctactataggtacgaggggggtatggactattggggtcaaggaacctcagtcaccgtctcctca

SEQ ID NO: 79 - protein, 107 aa, Mouse
DIKMTQSPSSMYASLGERVTITCKASQDINTYLNWFQQKPGKSPMTLIYRADRLLDGVPSRFSGSGSGQDYSLTISSLEDEDMGIYYCQQYDDFPLTFGAGTKLELK

SEQ ID NO: 80 - protein, 20 aa, Mouse
MVSSAQFLGILLLWFPGIKC

SEQ ID NO: 81 - DNA, 381 bp, Mouse
atggtatcctcagctcagttccttggaatcttgttgctctggtttccaggtatcaaatgtgacatcaagatgacccagtctccatcttccatgtatgcatctctaggagagagagtcactatcacttgcaaggcgagtcaggacattaatacctatttaaactggttccagcagaaaccagggaaatctcctatgaccctgatctatcgtgcagacagattgttagatggggtcccatcaaggttcagtggcagtggatctgggcaagattattctctcaccatcagcagcctggaggatgaggatatgggaatttactattgtcaacagtatgatgactttcctctcacgttcggtgctgggaccaagctggagctgaaa

SEQ ID NO: 82 - protein, 121 aa, Mouse
QVQLKQSGPGLVQPSQSLSITCTVSGFSLTDYGIHWIRQPPGKGLEWLGVIWSGGSTDHNAVFTSRLNISKDNSKSQVFFKMNSLEPDDTAMYYCARNRAYYRYEGGMDYWGQGTSVTVSS

SEQ ID NO: 83 - protein, 19 aa, Mouse
MAVLALLFCLVTFPSCVLS

SEQ ID NO: 84 - DNA, 420 bp, Mouse
atggctgtcttagcgctgctcttctgcctggtgacattcccaagctgtgtcctatcccaggtgcagctgaagcagtcaggacctggcctcgtgcagccctcacagagcctgtccatcacctgcacagtctctggtttctcattaactgactatggtatacactggattcgccagcctccaggaaagggtctggagtggctgggagtgatatggagtggtggaagcacagaccataatgctgtcttcacatccagactgaatatcagcaaggacaactccaagagtcaagttttctttaaaatgaacagtctggaacctgatgacacagccatgtactactgtgccagaaatagagcctactataggtacgaggggggtatggactactggggtcaaggaacctcagtcaccgtctcctca

SEQ ID NO: 85 - protein, 107 aa, Mouse; identical to SEQ ID NO: 79

SEQ ID NO: 86 - protein, 20 aa, Mouse
MVSSAQFLGILLLWFPGIKC

SEQ ID NO: 87 - DNA, 381 bp, Mouse
atggtatcctcagctcagttccttggaatcttgttgctctggtttccaggtatcaaatgtgacatcaagatgacccagtctccatcttccatgtatgcatctctaggagagagagtcactatcacttgcaaggcgagtcaggacattaatacctatttaaactggttccagcagaaaccagggaaatctcctatgaccctgatctatcgtgcagacagattgttagatggggtcccatcaaggttcagtggcagtggatctgggcaagattattctctcaccatcagcagcctggaggatgaagatatgggaatttactattgtcaacagtatgatgactttcctctcacgttcggtgctgggaccaagctggagctgaaa

SEQ ID NO: 88 - protein, 123 aa, Mouse
QVTLKESGPGILQPSQTLSLTCSFSGFSLSTSGMGVSWIRQPSGKGLEWLAHIYWDDDKRYNPSLRSRLTISKDTSRNQVFLKITSVDTADTATYYCARREGYYGSSFDFDVWGAGTTVTVSS

SEQ ID NO: 89 - protein, 19 aa, Mouse
MGRLTFSFLLLIVPAYVLS

SEQ ID NO: 90 - DNA, 426 bp, Mouse
atgggcagacttacattctcattcctgctgctgattgtccctgcatatgtcctttcccaggttaccctgaaagagtctggccctgggatattgcagccctcccagaccctcagtctgacttgttctttctctgggttttcactgagcacttctgggatgggtgtgagctggattcgtcagccttcaggaaagggtctggagtggctggcacacatttactgggatgatgacaagcgctataacccatccctgaggagccggctcacaatctccaaggatacctccagaaaccaggtattcctcaagatcaccagtgtggacactgcagatactgccacatactactgtgctcgaagagagggttactacggtagtagcttcgacttcgatgtctggggcgcagggaccacggtcaccgtctcctct

SEQ ID NO: 91 - protein, 106 aa, Mouse
QIVLSQSPAILSASPGEKVTMTCRASSSVSYMIWYQQKPGSSPKPWIYATSSLASGVPTRFSGSGSGTSYSLTISRVEAADAATYYCQQWSYNPLTFGAGTKLELK

SEQ ID NO: 92 - protein, 22 aa, Mouse
MDLQVQIFSFLLISASVKMSRG

SEQ ID NO: 93 - DNA, 384 bp, Mouse
atggatttacaggtgcagattttcagcttcctgctaatcagtgcttcagtcaaaatgtccagaggacaaattgttctctcccagtctccagcaatcctgtctgcatctccaggggagaaggtcacaatgacttgcagggccagctcaagtgtgagttacatgatctggtaccaacagaagccaggatcctcccccaaaccctggatttatgccacatccagcctggcttctggagtccctactcgcttcagtggcagtgggtctgggacctcttactctctcacaatcagcagagtggaggctgcagatgctgccacttattactgccagcagtggagttataacccgctcacgttcggtgctgggaccaagctggagctgaaa

SEQ ID NO: 94 - protein, 10 aa, Artificial Sequence
  Synthetic sequence
GFSLSTYGVH

SEQ ID NO: 95 - protein, 16 aa, Artificial Sequence
  Synthetic sequence
VIWRGGNTNYNAAFMS

SEQ ID NO: 96 - protein, 14 aa, Artificial Sequence
  Synthetic sequence
NQIYDGYYDYAMDY

SEQ ID NO: 97 - protein, 10 aa, Artificial Sequence
  Synthetic
GFSLTDYGIH

SEQ ID NO: 98 - protein, 16 aa, Artificial Sequence
  Synthetic sequence
VIWSGGSTDHNAVFTS

SEQ ID NO: 99 - protein, 13 aa, Artificial Sequence
  Synthetic sequence
NRAYYRYEGGMDY

SEQ ID NO: 100 - protein, 10 aa, Artificial Sequence
  Synthetic sequence
GFSLTNYGIH

SEQ ID NO: 101 - protein, 16 aa, Artificial Sequence
  Synthetic sequence
VIWSGGSTDHNAAFIS

SEQ ID NO: 102 - protein, 13 aa, Artificial Sequence
  Synthetic sequence
NRAYYRYEGGMDY

SEQ ID NO: 103 - protein, 12 aa, Artificial Sequence
  Synthetic sequence
GFSLSTSGMGVS

SEQ ID NO: 104 - protein, 13 aa, Artificial Sequence
  Synthetic sequence
REGYYGSSFDFDV

SEQ ID NO: 105 - protein, 12 aa, Artificial Sequence
  Synthetic sequence
GFSLXXXGXXXX

SEQ ID NO: 106 - protein, 16 aa, Artificial Sequence
  Synthetic sequence
VIWXGGXTXXNAXFXS

SEQ ID NO: 107 - protein, 9 aa, Artificial Sequence
  Synthetic sequence
DAYSYGMDY

SEQ ID NO: 108 - protein, 7 aa, Artificial Sequence
  Synthetic sequence
GYDAMDY

SEQ ID NO: 109 - protein, 10 aa, Artificial Sequence
  Synthetic sequence
GYSFTGYFMN

SEQ ID NO: 110 - protein, 17 aa, Artificial Sequence
  Synthetic sequence
RINPYNGDSFYNQKFKD

SEQ ID NO: 111 - protein, 10 aa, Artificial Sequence
  Synthetic sequence
GFTFSDYYMY

SEQ ID NO: 112 - protein, 17 aa, Artificial Sequence
  Synthetic sequence
TIGDGTSYTHYPDSVQG

SEQ ID NO: 113 - protein, 7 aa, Artificial Sequence
  Synthetic sequence
GTGPFAY

SEQ ID NO: 114 - protein, 10 aa, Artificial Sequence
  Synthetic sequence
GYVFSSSWMN

SEQ ID NO: 115 - protein, 17 aa, Artificial Sequence
  Synthetic sequence
RIYPGDGDTDYTGKFKG

SEQ ID NO: 116 - protein, 5 aa, Artificial Sequence
  Synthetic sequence
GYFDF

SEQ ID NO: 117 - protein, 11 aa, Artificial Sequence
  Synthetic sequence
GYSITSGYYWN

SEQ ID NO: 118 - protein, 17 aa, Artificial Sequence
  Synthetic sequence
GYISYDGSNNYNPSLKN

SEQ ID NO: 119 - protein, 13 aa, Artificial Sequence
  Synthetic sequence
HIYWDDDKRYNPS

SEQ ID NO: 120 - protein, 11 aa, Artificial Sequence
  Synthetic sequence
GXXXXXXXXXX

SEQ ID NO: 121 - protein, 11 aa, Artificial Sequence
  Synthetic sequence
KASQDINTYLN

SEQ ID NO: 122 - protein, 7 aa, Artificial Sequence
  Synthetic sequence
RANRLVD

SEQ ID NO: 123 - protein, 7 aa, Artificial Sequence
  Synthetic sequence
RADRLLD

SEQ ID NO: 124 - protein, 9 aa, Artificial Sequence
  Synthetic sequence
QQYDDFPLT

SEQ ID NO: 125 - protein, 11 aa, Artificial Sequence
  Synthetic sequence
RASQDISNFLT

SEQ ID NO: 126 - protein, 7 aa, Artificial Sequence
  Synthetic sequence
RANRLVD

SEQ ID NO: 127 - protein, 9 aa, Artificial Sequence
  Synthetic sequence
QQVSKFPWT

SEQ ID NO: 128 - protein, 11 aa, Artificial Sequence
  Synthetic sequence
KASQDMNNYLR

SEQ ID NO: 129 - protein, 7 aa, Artificial Sequence
  Synthetic sequence
RAXRLXD

SEQ ID NO: 130 - protein, 9 aa, Artificial Sequence
  Synthetic sequence
FQGSHFPWT

SEQ ID NO: 131 - protein, 11 aa, Artificial Sequence
  Synthetic sequence
XASQDXXXXLX

SEQ ID NO: 132 - protein, 9 aa, Artificial Sequence
  Synthetic sequence
QQSNKDPYT

SEQ ID NO: 133 - protein, 9 aa, Artificial Sequence
  Synthetic sequence
QQWSYNPLT

SEQ ID NO: 134 - protein, 15 aa, Artificial Sequence
  Synthetic sequence
RASESVDNYGISFMH

SEQ ID NO: 135 - protein, 7 aa, Artificial Sequence
  Synthetic sequence
YTSKLHS

SEQ ID NO: 136 - protein, 9 aa, Artificial Sequence
  Synthetic sequence
LHYDAFPWT

SEQ ID NO: 137 - protein, 10 aa, Artificial Sequence
  Synthetic sequence
RASSSVSYMI

SEQ ID NO: 138 - protein, 7 aa, Artificial Sequence
  Synthetic sequence
KVSNRFS

SEQ ID NO: 139 - protein, 9 aa, Artificial Sequence
  Synthetic sequence
LQHDKFPPT

SEQ ID NO: 140 - protein, 10 aa, Artificial Sequence
  Synthetic sequence
RASXSVXXXX

SEQ ID NO: 141 - protein, 7 aa, Artificial Sequence
  Synthetic sequence
RASNLES

SEQ ID NO: 142 - protein, 7 aa, Artificial Sequence
  Synthetic sequence
KASNLES

SEQ ID NO: 143 - protein, 7 aa, Artificial Sequence
  Synthetic sequence
RASSLES

SEQ ID NO: 144 - protein, 7 aa, Artificial Sequence
  Synthetic sequence
KASSLES

SEQ ID NO: 145 - protein, 7 aa, Artificial Sequence
  Synthetic sequence
ATSSLAS

SEQ ID NO: 146 - protein, 16 aa, Artificial Sequence
  Synthetic sequence
RSSQNIVHSNGNTYLE

SEQ ID NO: 147 - protein, 7 aa, Artificial Sequence
  Synthetic sequence
XXSXXXS

SEQ ID NO: 148 - protein, 9 aa, Artificial Sequence
  Synthetic sequence
LXXDXFPXT

SEQ ID NO: 149 - protein, 111 aa, Mouse; identical to SEQ ID NO: 73

SEQ ID NO: 150 - protein, 118 aa, Mouse; identical to SEQ ID NO: 70

SEQ ID NO: 151 - protein, 99 aa, Mouse
DIVLTQSPASLAVSLGQRATISCRASESVDNYGISFMHWYQQKPGQPPKLLIYRASNLESGIPARFSGSGSRTDFTLTINPVETDDVATFYCQQSNKDP

SEQ ID NO: 152 - protein, 94 aa, Artificial Sequence
  Synthetic sequence
DIVLTQSPASLAVSLGQRATISCRASESVDYGSFMHWYQQKPGQPPKLLIYRASNLESGIPARFSGSGSRTDFTLTINPVEDDVATYCQQSNDP

SEQ ID NO: 153 - protein, 99 aa, Mouse
DIVLTQSPASLAVSLGQRATISCRASESVDSYGNSFMHWYQQKPGQPPKLLIYRASNLESGIPARFSGSGSRTDFTLTINPVEADDVATYYCQQSNEDP

SEQ ID NO: 154 - protein, 98 aa, Mouse
DVQLQESGPGLVKPSQSLSLTCSVTGYSITSGYYWNWIRQFPGNKLEGMGYISYDGSNNYNPSLKNRISITRDSSKNQFFLKLNSVTAEDSGTYYCAR

SEQ ID NO: 155 - protein, 79 aa, Artificial Sequence
  Synthetic sequence
VQLQESGPLVKPSQLSLTCSVTGSITSYWNWIRFPGNKLEMGYISYGSYNPSLKRISITRDSKNQLLNSVTEDTYYCAR

SEQ ID NO: 156 - protein, 97 aa, Mouse
EVQLQESGPSLVKPSQTLSLTCSVTGDSITSDYWNWIRKFPGNKLEYMGYISYSGSTYYNPSLKSRISITRDTSKNQYYLQLNSVTSEDTATYYCAR

SEQ ID NO: 157 - protein, 99 aa, Homo sapiens; identical to SEQ ID NO: 151

SEQ ID NO: 158 - protein, 65 aa, Artificial Sequence
  Synthetic sequence
DIVTQSPSLAVSLGRATICSSVWYQQKPGQPPKLLIYASESGPRFSGSGSTDFTLTIDVAYCQQP

SEQ ID NO: 159 - protein, 101 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 160 - protein, 97 aa, Homo sapiens
VQLQESGPGLVKPSQSLSLTCSVTGYSITSGYYWNWIRQFPGNKLEGMGYISYDGSNNYNPSLKNRISITRDSSKNQFFLKLNSVTAEDSGTYYCAR

SEQ ID NO: 161 - protein, 66 aa, Artificial Sequence
  Synthetic sequence
VQLQESGPGLVKPSLSLTCVGYSISWWIRQPGLEGYIYGSYNPSLKRDSKNQFLKLSVTADYYCAR

SEQ ID NO: 162 - protein, 97 aa, Homo sapiens
VQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCAR

SEQ ID NO: 163 - protein, 88 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYC

SEQ ID NO: 164 - protein, 125 aa, Homo sapiens
EVQLVESGGGLVQPGGSLRLSCAASGFTFSNYEMNWVRQAPGKGLEWISYISNGDNTIYYADSVKGRFTISRDSAKNSLYLHMHSLRAEDTAVYYCARGDYGGNGYFYYYAMDVWGQGTTVTVSS

SEQ ID NO: 165 - protein, 112 aa, Mouse; identical to SEQ ID NO: 67

SEQ ID NO: 166 - protein, 118 aa, Mouse; identical to SEQ ID NO: 64

SEQ ID NO: 167 - protein, 100 aa, Mouse
DVLMTQTPRSLPVSLGDQASISCRSSQNIVHSNGNTYLEWYLQKPGQSPKLLIYKVSNRFSGVPDRFSGSGSGTDFTLKISRVEAEDLGVYYCFQGSHFP

SEQ ID NO: 168 - protein, 97 aa, Artificial Sequence
  Synthetic sequence
DVLMTQTPSLPVSLGDQASISCRSSQIVHSNGNTYLEWYLQKPGQSPKLLIYKVSNRFSGVPDRFSGSGSGTDFTLKISRVEAEDLGVYYCFQGSHP

SEQ ID NO: 169 - protein, 100 aa, Mouse
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVP

SEQ ID NO: 170 - protein, 98 aa, Mouse
EVKLVESGGGLVKPGGSLKLSCAVSGFTFSDYYMYWIRQTPEKRLEWVATIGDGTSYTHYPDSVQGRFTISRDYATNNLYLQMTSLRSEDTALYYCAR

SEQ ID NO: 171 - protein, 85 aa, Artificial Sequence
  Synthetic sequence
EVKLVESGGGLVKPGGSLKLSCASGFTFSYMWRQTPEKRLEWVATIGSYTYPDSVGRFTISRDANNLYLQMSLRSEDTALYYCAR

SEQ ID NO: 172 - protein, 98 aa, Mouse
EVKLVESGGGLVKPGGSLKLSCAASGFTFSSYGMSWVRQTPEKRLEWVATISGGGSYTYYPDSVKGRFTISRDNAKNNLYLQMSSLRSEDTALYYCAR

SEQ ID NO: 173 - protein, 76 aa, Artificial Sequence
  Synthetic sequence
DMTQPSLPVGASISCRSSQHSNGYLWYLQKPGQSPLLIYSNRSGVPDRFSGSGSGTDFTLKISRVEAEDGVYYCQP

SEQ ID NO: 174 - protein, 100 aa, Mouse
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 175 - protein, 72 aa, Artificial Sequence
  Synthetic sequence
EVLVESGGGLVKPGGSLLSCASGFTFSYMWRQPKLEWVISYYDSVGRFTISRDANLYLQMSLREDTAYYCAR

SEQ ID NO: 176 - protein, 98 aa, Mouse
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 177 - protein, 93 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYC

SEQ ID NO: 178 - protein, 10 aa, Artificial Sequence
  Synthetic sequence
FGQGTKVEIK

SEQ ID NO: 179 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 180 - protein, 11 aa, Artificial Sequence
  Synthetic sequence
WGQGTLVTVSS

SEQ ID NO: 181 - protein, 10 aa, Artificial Sequence
  Synthetic sequence
FGQGTKVEIK

(19) United States
(12) Patent Application Publication
(10) Pub. No.: US 2014/0079716 A1
(43) Pub. Date: Mar. 20, 2014

(54) ANTI-LYMPHOTOXIN ANTIBODIES

(71) Applicant: BIOGEN IDEC MA INC., (US)

(72) Inventors: Ann M. RANGER, Northborough, MA (US); Ellen GARBER, Cambridge, MA (US); Justin A. CARAVELLA, Cambridge, MA (US); Alexey A. LUGOVSKOY, Woburn, MA (US); Joseph ARNDT, Peabody, MA (US); Frederick R. TAYLOR, Milton, MA (US); Giovanna ANTOGNETTI, Foxboro, MA (US); Eric DAY, Cambridge, MA (US)

(73) Assignee: BIOGEN IDEC MA INC., Cambridge, MA (US)

(21) Appl. No.: 13/830,500

(22) Filed: Mar. 14, 2013

Related U.S. Application Data
(63) Continuation of application No. 13/141,297, filed on Jun. 21, 2011.
(60) Provisional application No. 61/142,182, filed on Dec. 31, 2008.

(51) Int. Cl. C07K 14/525

(52) U.S. Cl. CPC C07K 14/5255
USPC 424/158.1; 530/389.2; 536/23.53; 435/320.1; 435/252.3; 435/69.6; 435/252.33; 435/252.31; 435/254.2; 435/254.21; 435/254.23; 435/335

(57) ABSTRACT

The instant invention is based, at least in part on the identification of a new class of antibodies that result, e.g., in improved LT blocking capabilities. Methods of making the subject binding molecules and methods of using the binding molecules of the invention to antagonize LTβR signaling are also provided.

### RELATED APPLICATIONS

This application is a continuation of U.S. patent application Ser. No. 13/141,297, filed on Jun. 21, 2011 which is a U.S.C. §371 national stage filing of PCT Application No. PCT/US2009/069967 filed on Dec. 31, 2009, which claims priority to, and the benefit of, U.S. Provisional Application No. 61/142,182 filed Dec. 31, 2008. The contents of the aforementioned applications are hereby incorporated by reference.

### BACKGROUND OF THE INVENTION

Lymphotoxin (LT) is a cytokine related to TNF and which is found in human systems in both secreted and membrane bound forms. The secreted form is a trimer of a single protein, LT-α, whereas the surface form of LT is a complex of two related molecules, LT-α and LT-β. The predominant form is a heterotrimer having the composition α1β2, however, α2β1 heterotrimers also exist. The only known cell-surface receptors for the LTα homotrimer are the two TNF receptors, p55, p75, and HVEM. In contrast, the LT α1β2 heterotrimer does not bind to these TNF receptors, but rather to LTβ receptor (LTβR). The binding of LT to LTβR plays an important role in lymphoneogenesis and inflammation. The development of antibodies that potently and specifically block the binding of LT to LTβR would be of tremendous benefit in modulating LTβR-mediated responses in patients.

### SUMMARY OF THE INVENTION

LT α1β2 is a unique member of the TNF ligand family because it is a heterotrimer of two different chains LTα and LTβ, rather than a homotrimer of a single chain as found for other LT family members. The receptors for this family of molecules are found to bind in the clefts between the trimer chains and, if the ligand is a homotrimer, all three clefts are identical and a single antibody that binds in a cleft would be expected to block all three binding sites simultaneously. In contrast, the LTα1β2 heterotrimer presents three different clefts (that can be designated β-β, β-α, and α-β) and, until the instant invention, it was not clear that a single antibody could bind to the heterotrimer and block all sites of receptor binding effectively and, thereby, block biological activity completely. It is noteworthy that the instant antibodies do not bind to LTα3 (or bind to LTα3, but not in such a way as to block TNFα receptor binding) and have improved function as compared to anti-LT α1β2 antibodies of the prior art.

For example, in one embodiment, the instant antibodies more potently block the binding of LT to LTβR and/or more potently block one or more biological effects of LT-signaling via LTβR than the antibodies of the prior art (as used herein, the term LT refers to LT α1β2 unless otherwise indicated). For instance, in one embodiment, these antibodies result in greater than 70% blockade of LT-induced cytokine production. In another embodiment, these antibodies result in greater than 80% blockade of LT-induced cytokine production. In one embodiment, these antibodies result in greater than 90% blockade of LT-induced cytokine production. In one embodiment, these antibodies result in greater than 95% blockade of LT-induced cytokine production. In another embodiment, such antibodies have an IC50 for inhibition of LT binding and/or LT-induced cytokine production of less than approximately 0.05 ug/ml. In one embodiment, such antibodies have an IC50 for inhibition of LT binding and/or LT-induced cytokine production of less than approximately 100 nM. In one embodiment, such antibodies have an IC50 for inhibition of LT binding and/or LT-induced cytokine production of less than approximately 30 nM. In one embodiment, such antibodies have an IC50 for inhibition of LT binding and/or LT-induced cytokine production of less than approximately 10 nM. In one embodiment, such antibodies have an IC50 for inhibition of LT binding and/or LT-induced cytokine production of less than approximately 3 nM. A panel of such antibodies has been developed and the epitopes to which several of these antibodies bind have been mapped. In preferred embodiments, the antibodies of the instant invention also bind to epitopes of LT of non-human primates, e.g., cynomologous monkeys. The structure of the variable regions of these antibodies has also been elucidated. The CDRs from this panel of antibodies (e.g., Chothia or Kabat CDRs) can be used to generate binding molecules (e.g., humanized antibodies, modified antibodies, single chain binding molecules) that bind to LT and block LT-induced signaling. Accordingly, the instant invention is directed to binding molecules which comprise one or more binding sites (e.g., variable heavy and variable light regions) specific for LT, which block the binding of LT to LTβR, and which have improved functional properties when compared to the antibodies of the prior art.

In one aspect, the invention pertains to an isolated binding molecule that binds to lymphotoxin (LT) and blocks an LT-induced biological activity in a cell by at least about 70% under conditions in which a reference antibody, B9, (Produced by the cell line B9.C9.1, deposited with the ATCC under Accession number HB11962) blocks the LT-induced biological activity in a cell by about 50%, or a molecule comprising an antigen binding region thereof.

In another aspect, the invention pertains to an isolated binding molecule that binds to lymphotoxin (LT) and blocks an LT-induced biological activity in a cell at an IC50 of less than 100 nM or a molecule comprising an antigen binding region thereof.

In another aspect, the invention pertains to an isolated binding molecule that binds to lymphotoxin (LT) and blocks LTβR-Ig binding to a cell by at least 85% or a molecule comprising an antigen binding region thereof.

In another aspect, the invention pertains to an isolated binding molecule or molecule comprising an antigen binding region thereof, wherein the LT-induced biological activity is IL-8 release.

In one embodiment, the binding molecule comprises a human amino acid sequence.

In one embodiment, the binding molecule comprises an antigen binding region thereof comprises the human amino acid sequence comprises an antibody constant region sequence or fragment thereof.

In one embodiment, the invention pertains to binding molecule, wherein the human constant region is an IgG1 constant region that has been altered to reduce binding to at least one Fc receptor.

In one embodiment, the invention pertains to a binding molecule, wherein the human constant region is an IgG1 constant region that has been altered to enhance binding to at least one Fc receptor.

In one embodiment, the invention pertains to binding molecule which is humanized.

In one embodiment, the LT-induced biological activity is blocked by at least about 80%. In one embodiment, the LT-induced biological activity is blocked by at least about 90%. In one embodiment, LTBR-Ig-binding is blocked by at least about 90%.

In one embodiment, a binding molecule blocks an LT-induced biological activity in a cell at an IC50 of less than 30 nM or a molecule comprising an antigen binding region thereof.

In one embodiment, a binding molecule blocks an LT-induced biological activity in a cell at an IC50 of less than 10 nM or a molecule comprising an antigen binding region thereof.

In another embodiment, a binding molecule of the invention blocks an LT-induced biological activity in a cell at an IC50 of less than 3 nM or a molecule comprising an antigen binding region thereof.

In one embodiment, the binding molecule binds to two sites on LT leaving no site for LTβR binding.

In one embodiment, a binding molecule is a full length antibody. In one embodiment, a binding molecule is an scFv molecule.

In another embodiment, the invention pertains to a binding molecule that specifically binds to an epitope of LT, wherein the binding to the LT epitope by the antibody is competitively blocked in a dose-dependent manner by the 102 antibody.

In one embodiment, amino acids 193 and 194 of LTβ are critical for binding of the antibody.

In another embodiment, the invention pertains to a binding molecule that specifically binds to an epitope of LT, wherein the binding to the LT epitope by the antibody is competitively blocked in a dose-dependent manner by the AOD9 antibody.

In another embodiment, the invention pertains to a binding molecule that specifically binds to an epitope of LT, wherein the binding to the LT epitope by the antibody is competitively blocked in a dose-dependent manner by the 101/103 antibody.

In another embodiment, the invention pertains to a binding molecule that specifically binds to an epitope of LT, wherein the binding to the LT epitope by the antibody is competitively blocked in a dose-dependent manner by the 105 antibody.

In one embodiment, amino acids 96, 97, 98, 106, 107, and 108 of LTβ are critical for binding of the antibody.

In another embodiment, the invention pertains to a binding molecule that specifically binds to an epitope of LT, wherein the binding to the LT epitope by the antibody is competitively blocked in a dose-dependent manner by the 9B4 antibody.

In one embodiment, amino acids 96, 97, and 98 of LTβ are critical for binding of the antibody.

In another embodiment, the invention pertains to a binding molecule that specifically binds to an epitope of LT, wherein the binding to the LT epitope by the antibody is competitively blocked in a dose-dependent manner by the A1D5 antibody.

In one embodiment, amino acid 172 of LTβ is critical for binding of the antibody.

In another embodiment, the invention pertains to a binding molecule that specifically binds to an epitope of LT, wherein the binding to the LT epitope by the antibody is competitively blocked in a dose-dependent manner by the 107 antibody.

In another embodiment, the invention pertains to a binding molecule that specifically binds to an epitope of LT amino acids 151 and 153 of LTβ are critical for binding of the antibody.

In one embodiment, the invention pertains to an isolated antibody that specifically binds to an epitope of LT, wherein the binding to the LT epitope by the antibody is competitively blocked in a dose-dependent manner by the 108 antibody.

In one embodiment, the binding molecule comprises a human amino acid sequence.

In one embodiment, the human amino acid sequence is an antibody constant region sequence.

In one embodiment, the antibody is humanized.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the light and heavy chain CDRs are derived from an antibody selected from the group consisting of AOD9, 108, 107, A1D5, 102,101/103, 9B4 and 105.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the AOD9 antibody.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 108 antibody.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 107 antibody.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the A1D5 antibody.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 102 antibody.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 101/103 antibody.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 105 antibody.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 9B4 antibody.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein CDRH1 comprises the sequence GFSLX₁X₂Y/SGX₃H (SEQ ID NO: 1) wherein X is any amino acid.

In another aspect, the invention pertains to a lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein CDRH2 comprises the sequence VIWX₁GGX₂TX₃X₄NAX₅FX₆S (SEQ ID NO: 2), wherein X is any amino acid.

In another aspect, the invention pertains to a lymphotoxin binding molecule comprising a light chain variable region comprising heavy chain CDRs CDRH1, CDRH2 and CDRH3 and light chain variable region comprising light chain CDRs CDRL1, CDRL2, and CDRL3, wherein CDRL1 comprises the sequence RASX₁SVX₂X₃X₄X₅ (SEQ ID NO: 3) or X₁ASQDX₂X₃X₄X₅LX₆ (SEQ ID NO: 4) wherein X is any amino acid.

In another aspect, the invention pertains to a lymphotoxin binding molecule comprising a light chain variable region comprising heavy chain CDRs CDRH1, CDRH2 and CDRH3 and light chain variable region comprising light chain CDRs CDRL1, CDRL2, and CDRL3, wherein CDRL2 comprises the sequence RAX₁RLX₂D (SEQ ID NO: 5) wherein X is any amino acid.

In another aspect, the invention pertains to a lymphotoxin binding molecule comprising a light chain variable region comprising heavy chain CDRs CDRH1, CDRH2 and CDRH3 and light chain variable region comprising light chain CDRs CDRL1, CDRL2, and CDRL3, wherein CDRL2 comprises the sequence X₁X₂SX₃X₄X₅S (SEQ ID NO: 6) wherein X is any amino acid.

In another aspect, the invention pertains to a lymphotoxin binding molecule comprising a light chain variable region comprising heavy chain CDRs CDRH1, CDRH2 and CDRH3 and light chain variable region comprising light chain CDRs CDRL1, CDRL2, and CDRL3, wherein CDRL3 comprises the sequence X₁QX₂X₃X₄X₅PX₆T (SEQ ID NO: 7) wherein X is any amino acid.

In another aspect, the invention pertains to a lymphotoxin binding molecule comprising a light chain variable region comprising heavy chain CDRs CDRH1, CDRH2 and CDRH3 and light chain variable region comprising light chain CDRs CDRL1, CDRL2, and CDRL3, wherein CDRL3 comprises the sequence LX₁X₂DX₄FPX₆T (SEQ ID NO: 8) wherein X is any amino acid.

In another aspect, the invention pertains to a lymphotoxin binding molecule comprising a light chain variable region comprising heavy chain CDRs CDRH1, CDRH2 and CDRH3 of a 105 antibody variant and light chain variable region comprising light chain CDRs CDRL1, CDRL2, and CDRL3 of a 105 variant.

In one embodiment, the invention pertains to a binding molecule which has a solubility of greater than 100 or 120 mg/ml.

In one embodiment, the binding molecule comprises the light chain variable region of the 105 variant version L10.

In one embodiment, the binding molecule comprises the heavy chain variable region of the 105 variant version H1.

In one embodiment, the binding molecule comprises the heavy chain variable region of the 105 variant version H1 or the CDRs thereof and the light chain variable region of the 105 variant L10 or the CDRs thereof.

In one embodiment, the invention pertains to a composition comprising a binding molecule of the invention and a carrier.

In one embodiment, the invention pertains to a method of treating a subject that would benefit from treatment with an anti-LT binding molecule comprising administering the molecule to a subject such that treatment occurs.

In one embodiment, the subject is suffering from a disorder characterized by inflammation.

In one embodiment, the inflammatory disorder is selected from group consisting of rheumatoid arthritis, multiple sclerosis, Crohn's disease, ulcerative colitis, a transplant, lupus, inflammatory liver disease, psoriasis, Sjorgren's syndrome, multiple sclerosis (e.g., SPMS), viral-induced hepatitis, autoimmune hepatitis, type I diabetes, atherosclerosis, and viral shock syndrome.

In one embodiment, the inflammatory disorder is rheumatoid arthritis.

In one embodiment, the subject is suffering from cancer. In one embodiment, the cancer is selected from the group consisting of multiple myeloma and indolent follicular lymphoma.

In one aspect the invention pertains to a nucleic acid molecule encoding a binding molecule of the invention. In one embodiment, the nucleic acid molecule is in a vector.

In one embodiment, the invention pertains to a host cell comprising the vector.

In one embodiment, the invention pertains to a method of producing the antibody or binding molecule, comprising (i) culturing the host cell of claim 66 such that the antibody or binding molecule is secreted in host cell culture media (ii) isolating the antibody or binding molecule from the media.

In another aspect, the invention pertains to the use of a composition comprising a binding molecule of the invention in the manufacture of a medicament.

In another embodiment, the medicament is for the treatment of a disorder associated with inflammation.

### BRIEF DESCRIPTION OF THE DRAWINGS

FIGS. 1A, 1B and 1C show inhibition curves using an IL-8 release assay for anti-LT antibodies. In panel A, the open diamonds represent the 102 antibody, the open squares represent the 105 antibody, the closed triangles represent the A0D9 antibody, the open triangles represent the B9 antibody, the closed circles represent the C37 antibody, and the open circles represent the B27 antibody. In panel B the closed triangles represent the 105 antibody and the open triangles represent the 107 antibody. Panel C represents the inhibition curve for the 9B4 antibody.

FIGS. 2A-2G provide histological results showing status of MOMA-1+ macrophages from chimerized (huSCID) mice injected with MOPC-21 (murine IgG1 antibody used as isotype control): FIG. 2B), mLTBR-mIgG1 (FIG. 2C), antibody BBF6 (mIgG1) (FIG. 2D); antibody B9 (mIgG1) (FIG. 2E); antibody LT102 (FIG. 2F), antibody LT105 (FIG. 2G). Wild type C57BL/6 sections are also shown in FIG. 2A.

FIGS. 3A-3G provide histological results showing reduction in HEVs with blockade of human LTα1 β2. MOPC-21 (murine IgG1 antibody used as isotype control): FIG. 3B), mLTBR-mIgG1 (FIG. 3C), antibody BBF6 (mIgG1) (FIG. 3D); antibody B9 (mIgG1) (FIG. 3E); antibody LT102 (FIG. 3F), antibody LT105 (FIG. 3G). Wild type C57BL/6 sections are also shown in FIG. 3A.

FIGS. 4A-4B. Panel A provides a graph showing that antibodies LT102 and LT105 exhibit superior potency in a blocking assay which measures blocking of LTβRIg (or Fc) to cells which express LT. In panel A the closed squares represent LTβR-Ig, the open circles represent the 102 antibody, the open squares represent the 105 antibody, the open triangles represent the B9 antibody, the open diamonds represent the C37 antibody and the closed circles represent the B27 antibody. Panel B shows similar superior potency for blocking of LTβRIg (or Fc) by the antibody 9B4.

FIG. 5 provides data from an LTβRIg blocking assay (as in FIG. 4) showing that antibodies 102 (open triangles), 105 (closed circles), A1D5 (open diamonds), 107 (solid triangles), A0D9b (open circles), and 103 (solid diamonds) all block more effectively than B9 (open polygons) B27 (open reverse triangles), and C37 (open squares). LTbR is shown in solid squares.

FIG. 6 shows a schematic of the LT α1β2 heterotrimer including the three different clefts (αβ, βα, and ββ), including the two B subunits and the single A subunit.

### DETAILED DESCRIPTION OF THE INVENTION

### I. Definitions

It is to be noted that the term “a” or “an” entity refers to one or more of that entity; for example, “an LT binding molecule,” is understood to represent one or more LT binding molecules. (As used herein, the term LT refers to LT α1β2 unless otherwise indicated). As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be isolated or purified from a natural biological source or produced by recombinant technology, but is not necessarily translated from a designated nucleic acid sequence. It may be generated using methods known in the art, including by chemical synthesis.

A polypeptide of the invention comprises at least one binding site specific for LT as described in more detail herein. Accordingly, the subject polypeptides are also referred to herein as “binding molecules.” In one embodiment, a binding molecule of the invention is an anti-LT antibody or modified antibody.

In one embodiment, a polypeptide of the invention is isolated. An “isolated” polypeptide or a fragment, variant, or derivative thereof refers to a polypeptide that is not in its natural milieu. In one embodiment, no particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

As used herein the term “derived from” a designated protein refers to the origin of the polypeptide. In one embodiment, the polypeptide or amino acid sequence which is derived from a particular starting polypeptide is a variable region sequence (e.g. a VH and/or VL) or sequence related thereto (e.g. a CDR or framework region derived therefrom). In one embodiment, the amino acid sequence which is derived from a particular starting polypeptide is not contiguous. For example, in one embodiment, one, two, three, four, five, or six CDRs (e.g., Chothia or Kabat CDRs) are derived from a starting anti-LT antibody for use in a binding molecule of the invention. In one embodiment, the polypeptide or amino acid sequence that is derived from a particular starting polypeptide or amino acid sequence has an amino acid sequence that is essentially identical to that of the starting sequence or a portion thereof, wherein the portion consists of at least 3-5 amino acids, 5-10 amino acids, at least 10-20 amino acids, at least 20-30 amino acids, or at least 30-50 amino acids, or which is otherwise identifiable to one of ordinary skill in the art as having its origin in the starting sequence.

Also included as polypeptides of the present invention are fragments, derivatives, analogs, or variants of the foregoing polypeptides, and combinations thereof. The terms “fragment,” “variant,” “derivative” and “analog” when referring to binding molecules of the present invention include polypeptides which retain at least some of the binding properties of the corresponding molecule. Fragments of polypeptides of the present invention include proteolytic fragments, as well as deletion fragments, in addition to specific antibody fragments discussed elsewhere herein. Variants of binding molecules of the present invention include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Thus, an amino acid residue in a polypeptide may be replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of the polypeptide.

In one embodiment, the polypeptides of the invention are antibody molecules or modified antibody molecules that comprise at least one anti-LT antibody binding site comprising six CDRs (i.e., three light chain CDRs derived from an antibody that binds to LT and three heavy chain CDRs derived from the same or a different antibody that binds to LT). In one embodiment, a binding molecule of the invention comprises one binding site comprising a light chain variable region derived from an antibody that binds to LT and a heavy chain variable region derived from an antibody that binds to LT. In one embodiment, a binding molecule of the invention comprises at least two binding sites. In one embodiment, the binding molecule comprises two binding sites. In one embodiment, the binding molecule comprises more than two binding sites. In one embodiment, the invention pertains to these isolated LT binding molecules or the nucleic acid molecules which encode them.

In one embodiment, the binding molecules of the invention are monomers. In another embodiment, the binding molecules of the invention are multimers. For example, in one embodiment, the binding molecules of the invention are dimers. In one embodiment, the dimers of the invention are homodimers, comprising two identical monomeric subunits. In another embodiment, the dimers of the invention are heterodimers, comprising two non-identical monomeric subunits. The subunits of the dimer may comprise one or more polypeptide chains. For example, in one embodiment, the dimers comprise at least two polypeptide chains. In one embodiment, the dimers comprise two polypeptide chains. In another embodiment, the dimers comprise four polypeptide chains (e.g., as in the case of antibody molecules).

In one embodiment, the binding molecules of the invention are monovalent, i.e., comprise one LT target binding site (e.g., as in the case of a scFv molecule). In one embodiment, the binding molecules of the invention are multivalent, i.e., comprise more than one target binding site. In another embodiment, the binding molecules comprise at least two binding sites. In one embodiment, the binding molecules comprise two binding sites (e.g., as in the case of an antibody). In one embodiment, the binding molecules comprise three binding sites. In another embodiment, the binding molecules comprise four binding sites. In another embodiment, the binding molecules comprise greater than four binding sites.

As used herein the term “valency” refers to the number of potential binding sites in a binding molecule. A binding molecule may be “monovalent” and have a single binding site or a binding molecule may be “multivalent” (e.g., bivalent, trivalent, tetravalent, or greater valency). Each binding site specifically binds one target molecule or specific site on a target molecule (e.g., an epitope). When a binding molecule comprises more than one target binding site (i.e. a multivalent binding molecule), each target binding site may specifically bind the same or different molecules (e.g., may bind to different LT molecules or to different epitopes on the same molecule).

As used herein, the term “binding moiety”, “binding site”, or “binding domain” refers to the portion of an antibody variable region that specifically binds to LT. In one embodiment, the binding site comprises three light chain CDRs derived from an antibody that binds to LT and three heavy chain CDRs derived from an antibody that binds to LT.

The term “binding specificity” or “specificity” refers to the ability of a binding molecule to specifically bind (e.g., immunoreacts with) a given target molecule or epitope. In certain embodiments, the binding molecules of the invention comprise two or more binding specificities (i.e., they bind two or more different epitopes present on one or more different antigens at the same time). A binding molecule may be “mono specific” and have a single binding specificity or a binding molecule may be “multispecific” (e.g., bispecific or trispecific or of greater multispecificity) and have two or more binding specificities. In exemplary embodiments, the binding molecules of the invention are “bispecific” and comprise two binding specificities. Thus, whether an LT binding molecule is “monospecific” or “multispecific,” e.g., “bispecific,” refers to the number of different epitopes with which a binding molecule reacts. In exemplary embodiments, multispecific binding molecules of the invention may be specific for different epitopes on one or more LT molecule. A given binding molecule of the invention may be monovalent or multivalent for a particular binding specificity.

Binding molecules disclosed herein may be described or specified in terms of the epitope(s) or portion(s) of an antigen, e.g., an LT target polypeptide) that they recognize or to which they specifically bind. The portion of a target polypeptide which specifically interacts with the binding site or moiety of a binding molecule is an “epitope,” or an “antigenic determinant.” A target polypeptide may comprise a single epitope, but typically comprises at least two epitopes, and can include a number of epitopes, depending on the size, conformation, and type of antigen. Furthermore, it should be noted that an “epitope” on a target polypeptide may be or may include non-polypeptide elements, e.g., an “epitope” may include a carbohydrate side chain. The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since CDRs can recognize an antigenic peptide or polypeptide in its tertiary form, the amino acids comprising an epitope need not be contiguous, and in some cases, may not even be on the same peptide chain. In the present invention, peptide or polypeptide epitope recognized by an anti-LT antibodies of the present invention contains a sequence of at least 4, at least 5, at least 6, at least 7, more preferably at least 8, at least 9, at least 10, at least 15, at least 20, at least 25, or between about 15 to about 30 contiguous or non-contiguous amino acids of LT. In one embodiment, a binding molecule of the invention binds bivalently to an LT heterotrimer. In one embodiment, a binding molecule of the invention binds to an LT heterotrimer such that the binding of the LTβR ligand by the heterotrimer is blocked, e.g., such that no binding sites for the LTβR ligand remain.

By “specifically binds,” it is generally meant that a binding molecule binds to an epitope via a binding site of the binding molecule (e.g., antigen binding domain), and that the binding entails some complementarity between that binding site and the epitope. According to this definition, a binding molecule is said to “specifically bind” to an epitope when it binds to that epitope, via the binding site, more readily than it would bind to an unrelated epitope. Where a binding molecule is multispecific, the binding molecule may specifically bind to a second epitope (ie., unrelated to the first epitope) via another binding site (e.g., antigen binding domain) of the binding molecule.

By “preferentially binds,” it is meant that the binding molecule specifically binds to an epitope via a binding site more readily than it would bind to a related, similar, homologous, or analogous epitope. Thus, an antibody which “preferentially binds” to a given epitope would more likely bind to that epitope than to a related epitope, even though such a binding molecule may cross-react with the related epitope.

As used herein, the term “cross-reactivity” refers to the ability of a binding molecule, specific for one antigen or antibody, to react with a second antigen and is a measure of relatedness between two different antigenic substances. Thus, an antibody is cross reactive if it binds to an epitope other than the one that induced its formation. The cross reactive epitope generally contains many of the same complementary structural features as the inducing epitope.

For example, certain binding molecules have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain antigen or epitope, if it does not bind any other analog, ortholog, or homolog of that antigen or epitope.

As used herein, the term “affinity” refers to a measure of the strength of the binding of an individual epitope with the binding site of a binding molecule. See, e.g., Harlow et al., Antibodies: A Laboratory Manual, (Cold Spring Harbor Laboratory Press, 2nd ed. 1988) at pages 27-28. Preferred binding affinities include those with a dissociation constant or Kd less t In one embodiment, a binding molecule of the invention specifically binds to LT with an affinity of less than 5×10⁻²M, 10⁻²M, 5×10⁻³M, 10⁻³M, 5×10⁻⁴M, 10⁻⁴M, 5×10⁻⁵M, 10⁻⁵M, 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹² M, 10⁻¹²M, 5×10⁻¹³M, 10⁻¹³M, 5×10⁻¹⁴M, 10⁻¹⁴M, 5×10⁻¹⁵M, or 10⁻¹⁵M. In one embodiment, a binding molecule of the invention binds to a high affinity site on an LT heterotrimer with an affinity of less than 100×10⁻⁹.

As used herein, the term “avidity” refers to the overall stability of the complex between a population of binding molecules (e.g. antibodies) and an antigen, that is, the functional combining strength of a binding molecule mixture with the antigen. See, e.g., Harlow at pages 29-34. Avidity is related to both the affinity of individual binding molecules in the population with specific epitopes, and also the valencies of the binding molecules and the antigen. For example, the interaction between a bivalent monoclonal antibody and an antigen with a highly repeating epitope structure, such as a polymer, would be one of high avidity.

As used herein the term “potency” refers to the concentration of a binding molecule which is found to give a certain level of efficacy in a particular assay. For example, in one embodiment, the subject binding molecules block a biological activity of LTβR by at least about 70%, at least 80%, or at least 90%; block LTbR binding by at least 80%, at least 90%, at least 95%, and/or block an LT-induced biological activity in a cell at an IC50 of less than 500 nM, less than 100 nM, less than 30 nM, less than 10 nM, less than 3 nM.

A binding site of a binding molecule of the invention comprises an antigen binding site of an antibody molecule. An antigen binding site is formed by variable regions that vary from one polypeptide to another. In one embodiment, the polypeptides of the invention comprise at least two antigen binding sites. As used herein, the term “antigen binding site” includes a site that specifically binds (immunoreacts with) an antigen (e.g., a cell surface or soluble form of an antigen). An antigen binding site includes an immunoglobulin heavy chain and light chain variable region and the binding site formed by these variable regions determines the specificity of the antibody. In one embodiment, an antigen binding site of the invention comprises at least one heavy or light chain CDR of an anti-LT antibody molecule. In another embodiment, an antigen binding site of the invention comprises at least two CDRs from one or more anti-LT antibody molecules. In another embodiment, an antigen binding site of the invention comprises at least three CDRs from one or more anti-LT antibody molecules. In another embodiment, an antigen binding site of the invention comprises at least four CDRs from one or more anti-LT antibody molecules. In another embodiment, an antigen binding site of the invention comprises at least five CDRs from one or more anti-LT antibody molecules. In another embodiment, an antigen binding site of the invention comprises at least six CDRs (three heavy and three light) from one or more antibody molecules that bind to LT.

Preferred binding molecules of the invention comprise framework and/or constant region amino acid sequences derived from a human amino acid sequence. However, binding polypeptides may comprise framework and/or constant region sequences derived from another mammalian species. For example, binding molecules comprising murine sequences may be appropriate for certain applications. In one embodiment, a primate framework region (e.g., non-human primate), heavy chain portion, and/or hinge portion may be included in the subject binding molecules. In one embodiment, one or more non-human (e.g., murine) amino acids may be present in the framework region of a binding polypeptide, e.g., a human or non-human primate framework amino acid sequence may comprise one or more amino acid back mutations in which the corresponding murine amino acid residue is present and/or may comprise one or mutations to a different amino acid residue not found in the starting murine antibody (e.g., other mutations which optimize binding or biophysical properties). Preferred binding molecules of the invention are less immunogenic in humans than are murine antibodies comprising the same CDRs.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, β, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention.

Light chains are classified as either kappa or lambda (κ, λ). Each heavy chain class may be bound with either a kappa or lambda light chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs), of an antibody (e.g., in some instances a CH3 domain) combine to form the variable region that defines a three dimensional antigen binding site. This quaternary antibody structure forms the antigen binding site present at the end of each arm of the Y. In one embodiment, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

As used herein the term “variable region CDR amino acid residues” includes amino acids in a CDR or complementarity determining region as identified using sequence or structure based methods. As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other and one of ordinary skill in the art could readily identify the CDRs of the anti-LT antibodies described herein using any of these definitions. The amino acid residues which encompass the CDRs as defined by each of the above cited references are set forth in below for comparison. Preferably, the term “CDR” is a CDR as defined by Kabat based on sequence comparisons.

CDR Definitions

CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein the term “variable region framework (FR) amino acid residues” refers to those amino acids in the framework region of an Ig chain or portion thereof. The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable region and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments, the CDRs are as defined by Kabat. In another embodiment, the CDRs are as defined by Chothia.

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of the variable region of an LTβR antibody or antigen-binding fragment, variant, or derivative thereof of the present invention are according to the Kabat numbering system.

As used herein, the term “Fc region” refers to the portion of an immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge domain, a CH2 domain, and a CH3 domain. Fc regions of antibody molecules are dimeric. Binding molecules of the invention may comprise a complete Fc region or one or more Fc moieties. In one embodiment, an Fc region of a binding molecule may be chimeric. For example, an Fc domain of a polypeptide may comprise a CH1 domain derived from an IgG1 molecule and a hinge region derived from an IgG3 molecule. In another example, an Fc region can comprise a hinge region derived, in part, from an IgG1 molecule and, in part, from an IgG3 molecule. In another example, an Fc region can comprise a chimeric hinge derived, in part, from an IgG1 molecule and, in part, from an IgG4 molecule. In one embodiment, a dimeric Fc region of the invention may comprise one polypeptide chain. In another embodiment, a dimeric Fc region of the invention may comprise two polypeptide chains, e.g., as in the case of an antibody molecule.

In one embodiment, a binding molecule of the invention comprises at least one constant region, e.g., a heavy chain constant region and/or a light chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Exemplary modifications include additions, deletions or substitutions of one or more amino acids in one or more domains. Such changes may be included to optimize effector function, half-life, etc.

Amino acid positions in a heavy chain constant region, including amino acid positions in the CH1, hinge, CH2, and CH3 domains, are numbered herein according to the EU index numbering system (see Kabat et al., in “Sequences of Proteins of Immunological Interest”, U.S. Dept. Health and Human Services, 5^{th} edition, 1991). In contrast, amino acid positions in a light chain constant region (e.g. CL domains) are numbered herein according to the Kabat index numbering system (see Kabat et al., ibid).

Exemplary binding molecules include or may comprise, for example, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Binding molecules of the invention which comprise an Ig heavy chain may be of any type (e.g., IgG, IgE, IgM, IgD, IgA, and IgY), class (e.g., IgG1, IgG2, IgG3, IgG4, IgA1 and IgA2) or subclass of immunoglobulin molecule.

Binding molecules may comprise the variable region(s) alone or in combination with the entirety or a portion of the following: hinge region, CH1, CH2, and CH3 domains. Also included in the invention are antigen-binding fragments comprising a combination of variable region(s) with a hinge region, CH1, CH2, and CH3 domains.

The term “fragment” refers to a part or portion of a polypeptide (e.g., an antibody or an antibody chain) comprising fewer amino acid residues than an intact or complete polypeptide. The term “antigen-binding fragment” refers to a polypeptide fragment of an immunoglobulin or antibody that binds antigen or competes with intact antibody (i.e., with the intact antibody from which they were derived) for antigen binding (i.e., specific binding). As used herein, the term “antigen binding fragment” of an antibody molecule includes antigen-binding fragments of antibodies, for example, an antibody light chain (VL), an antibody heavy chain (VH), a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein, the term “CH1 domain” includes the first (most amino terminal) constant region domain of an immunoglobulin heavy chain that extends, e.g., from about EU positions 118-215. The CH1 domain is adjacent to the V_{H} domain and amino terminal to the hinge region of an immunoglobulin heavy chain molecule, and does not form a part of the Fc region of an immunoglobulin heavy chain. In one embodiment, a binding molecule of the invention comprises a CH1 domain derived from an immunoglobulin heavy chain molecule (e.g., a human IgG1 or IgG4 molecule).

As used herein, the term “CH2 domain” includes the portion of a heavy chain immunoglobulin molecule that extends, e.g., from about EU positions 231-340. The CH2 domain is unique in that it is not closely paired with another domain. Rather, two N-linked branched carbohydrate chains are interposed between the two CH2 domains of an intact native IgG molecule. In one embodiment, a binding molecule of the invention comprises a CH2 domain derived from an IgG1 molecule (e.g. a human IgG1 molecule). In another embodiment, an altered polypeptide of the invention comprises a CH2 domain derived from an IgG4 molecule (e.g., a human IgG4 molecule). In an exemplary embodiment, a polypeptide of the invention comprises a CH2 domain (EU positions 231-340), or a portion thereof.

As used herein, the term “CH3 domain” includes the portion of a heavy chain immunoglobulin molecule that extends approximately 110 residues from N-terminus of the CH2 domain, e.g., from about position 341-446b (EU numbering system). The CH3 domain typically forms the C-terminal portion of the antibody. In some immunoglobulins, however, additional domains may extend from CH3 domain to form the C-terminal portion of the molecule (e.g. the CH4 domain in the μ chain of IgM and the ε chain of IgE). In one embodiment, a binding molecule of the invention comprises a CH3 domain derived from an IgG1 molecule (e.g., a human IgG1 molecule). In another embodiment, a binding molecule of the invention comprises a CH3 domain derived from an IgG4 molecule (e.g., a human IgG4 molecule).

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein, the term “chimeric antibody” refers to an antibody wherein the binding site or moiety (e.g., the variable region) is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein the term “scFv molecule” includes binding molecules which consist essentially of one light chain variable domain (VL) or portion thereof, and one heavy chain variable domain (VH) or portion thereof, wherein each variable domain (or portion thereof) is derived from the same or different antibodies. scFv molecules preferably comprise an scFv linker interposed between the VH domain and the VL domain. scFv molecules are known in the art and are described, e.g., in U.S. Pat. No. 5,892,019, Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837. The VL and VH domains of an scFv molecule are derived from one or more antibody molecules. It will also be understood by one of ordinary skill in the art that the variable regions of the scFv molecules of the invention may be modified such that they vary in amino acid sequence from the antibody molecule from which they were derived. For example, in one embodiment, nucleotide or amino acid substitutions leading to conservative substitutions or changes at amino acid residues may be made (e.g., in CDR and/or framework residues). Alternatively or in addition, mutations may be made to CDR amino acid residues to optimize antigen binding using art recognized techniques. The binding molecules of the invention maintain the ability to bind to LT antigen.

A “scFv linker” as used herein refers to a moiety interposed between the VL and VH domains of the scFv. scFv linkers preferably maintain the scFv molecule in a antigen binding conformation. In one embodiment, an scFv linker comprises or consists of an scFv linker peptide. In certain embodiments, an scFv linker peptide comprises or consists of a gly-ser connecting peptide. In other embodiments, an scFv linker comprises a disulfide bond.

As used herein, the term “gly-ser connecting peptide” refers to a peptide that consists of glycine and serine residues. An exemplary gly/ser connecting peptide comprises the amino acid sequence (Gly₄ Ser)ₙ (SEQ ID NO: 9). In one embodiment, n=1. In one embodiment, n=2. In another embodiment, n=3. In a preferred embodiment, n=4, i.e., (Gly₄ Ser)₄ (SEQ ID NO: 10). In another embodiment, n=5. In yet another embodiment, n=6. Another exemplary gly/ser connecting peptide comprises the amino acid sequence Ser(Gly₄Ser)ₙ (SEQ ID NO: 11). In one embodiment, n=1. In one embodiment, n=2. In a preferred embodiment, n=3. In another embodiment, n=4. In another embodiment, n=5. In yet another embodiment, n=6.

In one embodiment, a binding molecule of the invention is an engineered antibody molecule. As used herein, the term “engineered antibody” or “modified antibody” refers to a binding molecule comprising an anti-LT antibody binding site, but which is not a traditional bivalent, four chain, antibody molecule.

In one embodiment, such a molecule comprises a variable region in which the variable domain in either the heavy and light chain or both is altered by at least partial replacement of one or more CDRs (e.g., Kabat or Chothia CDRs) from an antibody of known specificity and, if necessary, by partial framework region replacement and sequence changing. In one embodiment, the CDRs may be derived from an antibody of the same class or even subclass as the antibody from which the framework regions are derived. In one embodiment, the CDRs are derived from an antibody of different class and preferably from an antibody from a different species. An engineered antibody in which one or more “donor” CDRs from a non-human antibody of known specificity are grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. In one embodiment such a “humanized” antibody may comprise additional changes, e.g., mutations of framework region amino acid sequences (such as backmutations to donor amino acid, mutation to germline amino acid, or other substitution). Given the explanations set forth herein and known in the art (e.g., U.S. Pat. Nos. 5,585,089, 5,693,761, 5,693,762, and 6,180,370) it will be well within the competence of those skilled in the art, either by carrying out routine experimentation or by trial and error testing to obtain a functional engineered or humanized antibody.

The term “polynucleotide” includes an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid molecule” includes one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an LT binding molecule contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid molecule which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an LT binding molecule or fragment, variant, or derivative thereof. Heterologous coding regions include without limitation specialized elements or motifs, such as a secretory signal peptide or a heterologous functional domain.

As used herein the term “engineered” with reference to nucleic acid or polypeptide molecules refers to such molecules manipulated by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

As used herein, the terms “treat” or “treatment” refer to both therapeutic treatment and prophylactic or preventative measures, wherein the object is to prevent or slow down (lessen) an undesired physiological change or disorder, such as the development or spread of inflammation. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

As used herein, phrases such as “a subject that would benefit from administration of a binding molecule” and “an animal in need of treatment” includes subjects, such as mammalian subjects, that would benefit from administration of a binding molecule used, e.g., for detection of an antigen recognized by a binding molecule (e.g., for a diagnostic procedure) and/or from treatment, i.e., palliation or prevention of a disease such as an inflammatory disease or cancer, with a binding molecule which specifically binds LT. As described in more detail herein, the binding molecule can be used in unconjugated form or can be conjugated, e.g., to a drug, prodrug, or an isotope.

As used herein the term “disorder characterized by inflammation” refers to a disorder cause or characterized by an inflammatory response in a subject. Inflammatory disorders can be acute or chronic. Exemplary inflammatory disorders include rheumatoid arthritis, multiple sclerosis, Crohn's disease, ulcerative colitis, a transplant, lupus, inflammatory liver disease, psoriasis, Sjorgren's syndrome, multiple sclerosis (e.g., SPMS), viral-induced hepatitis, autoimmune hepatitis, type I diabetes, atherosclerosis, and viral shock syndrome, and individuals about to undergo transplantation or which have undergone transplantation.

### II. Anti-LT Binding Molecules

A panel of novel anti-LT binding molecules has been developed. The anti-LT binding molecules of the invention display improved functional properties as compared to the antibodies of the prior art. In another embodiment, the anti-LT binding molecules of the invention have unique structural properties compared to the anti-LT antibodies of the prior art.

In one embodiment, the invention pertains to an antibody AOD9, 108, 107, 105, 9B4, A1D5, 102, or 101/103 antibody described herein (also referred to herein as LT antibodies (e.g., LT105); the CDRs of these antibodies; the variable region sequences of these antibodies; the CDR sequences of variant forms of these antibodies; the variable regions sequences of variant forms of these antibodies; and binding molecules comprising these CDRs and/or variable regions. Nucleic acid molecules encoding these binding molecules are also provided for. In certain embodiments, the invention pertains to mature forms of molecules lacking signal sequences. The functional and structural characteristics or the subject antibodies and other aspects of the invention are set forth in more detail below.

A. Increased Inhibition of LT-Induced Signaling

LT-induced signaling (upon binding to LTβR) induces inflammatory responses and is also involved in normal development of lymphoid tissue. The binding molecules of the invention compete with the LTβR for binding to lymphotoxin, thereby inhibiting LT-mediated signaling and reducing the LT mediated biological response in a cell. A variety of assays may be used to demonstrate the blocking effects of a binding molecule of the invention.

For instance, in one embodiment, the ability of a binding molecule of the invention to inhibit the binding of LT (e.g., an LT heterotrimer) to LTβR can be measured. In one embodiment, the physiological, monomeric LTβ receptor (LTβR) can be used. In a preferred embodiment, a dimeric form of the LTβ receptor, e.g., an LTBR-Ig fusion protein (Fc fusion protein such as has been described in the art) can be used in the blocking studies using methods known in the art or described here. For example, biotin labeled LTβR will bind to lymphotoxin on II-23 cells treated with phorbol ester (PMA) which express LTα1β2 on their surface. The phorbol ester treated cells are are incubated with a binding molecule in competition with biotin labeled LTβR-Ig, the cells are washed to remove unbound LTβR-Ig, and the bound LTβR-Ig, is detected with streptavidin-PE. Thus, the ability of the binding molecule to block the binding of biotin tagged LTβR-Ig fusion protein to the surface LT (as compared to an appropriate control, e.g., the absence of the binding molecule) can be measured, e.g., using FACS analysis.

In another embodiment, the ability of a binding molecule to inhibit the production of a cytokine (e.g., IL-8) by LTβR expressing cells (e.g., A375 cells) is measured. In this assay LTβR expressing cells are contacted with LTα1β2 and a binding molecule and the ability of the binding molecule to inhibit IL-8 release by the cells (as compared to an appropriate control, e.g., the absence of the binding molecule) is measured, e.g., using an ELISA assay.

In one embodiment, a binding molecule of the invention achieves greater than 70% inhibition LTβR-Ig binding and/or inhibition of one or more LT biological activities, e.g., cytokine (such as IL-8) production. In one embodiment, a binding molecule of the invention achieves greater than 80% inhibition of LTβR-Ig binding and/or inhibition of one or more LT biological activities. In one embodiment, a binding molecule of the invention achieves greater than 90% inhibition of LTβR-Ig binding and/or inhibition of one or more LT biological activities. In one embodiment, a binding molecule of the invention achieves greater than 95% inhibition of LTβR-Ig binding and/or inhibition of one or more LT biological activities. In one embodiment, a binding molecule of the invention achieves complete (i.e., 100%) inhibition of LTβR-Ig binding and/or inhibition of one or more LT biological activities.

In one embodiment, the invention pertains to an isolated binding molecule that binds to lymphotoxin α1β2 and inhibits an LT α1β2-induced biological activity in a cell by at least about 70% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In another embodiment, an isolated binding molecule of the invention blocks an LT α1β2-induced biological activity in a cell by at least about 80% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In another embodiment, an isolated binding molecule of the invention blocks an LT α1β2-induced biological activity in a cell by at least about 85% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In another embodiment, an isolated binding molecule of the invention blocks an LT α1β2-induced biological activity in a cell by at least about 90% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In another embodiment, an isolated binding molecule of the invention blocks an LT α1β2-induced biological activity in a cell by at least about 95% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In another embodiment, an isolated binding molecule of the invention bocks an LT α1β2-induced biological activity in a cell by at least about 98% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In another embodiment, an isolated binding molecule of the invention bocks an LT α1β2-induced biological activity in a cell by at least about 100% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In one embodiment, the biological activity is IL-8 release.

In one embodiment, the invention pertains to an isolated binding molecule that binds to lymphotoxin β and inhibits an LTβR binding (or, as set forth above, dimeric LTBR-Ig binding) to a cell by at least about 70%. In another embodiment, the invention pertains to an isolated binding molecule that binds to lymphotoxin β and inhibits an LTβR (or LTBR-Ig) binding to a cell by at least about 80%. In another embodiment, the invention pertains to an isolated binding molecule that binds to lymphotoxin β and inhibits LTβR (or LTBR-Ig) binding to a cell by at least about 90%. In another embodiment, the invention pertains to an isolated binding molecule that binds to lymphotoxin β and inhibits LTβR (or LTBR-Ig) binding to a cell by at least about 95%. In another embodiment, the invention pertains to an isolated binding molecule that binds to lymphotoxin β and inhibits LTβR (or LTBR-Ig) binding to a cell by at least about 98%. In another embodiment, an isolated binding molecule of the invention pertains to an isolated binding molecule that binds to lymphotoxin β and inhibits LTβR binding to a cell by at least about 100% (or LTBR-Ig).

B. Increased Potency and/or Affinity

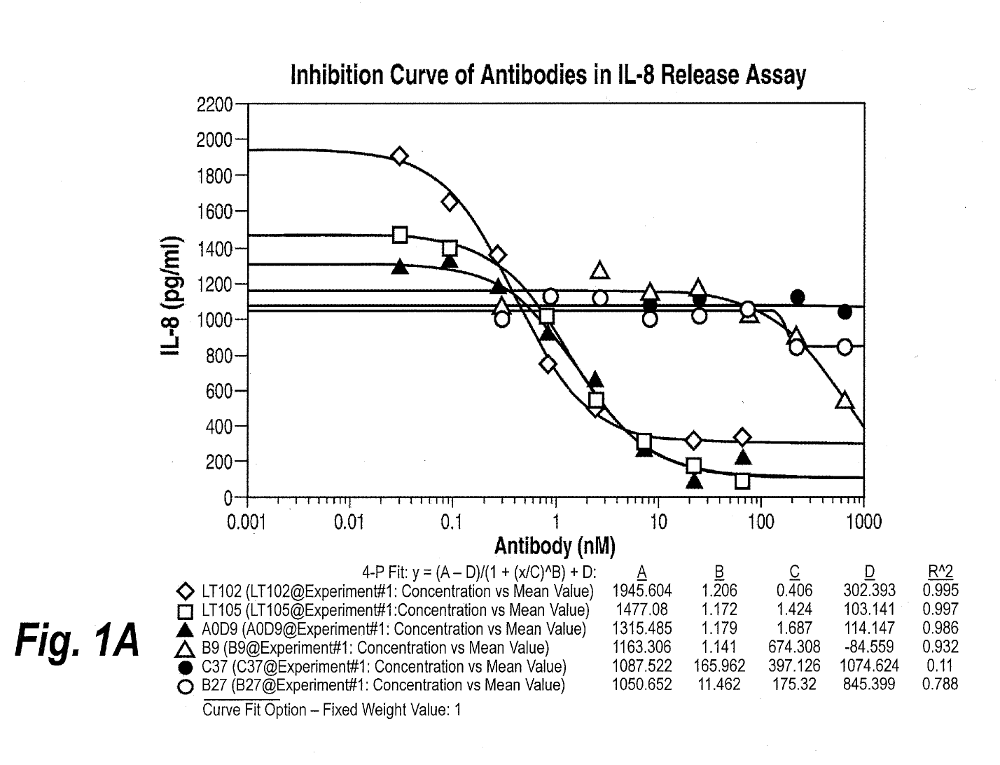

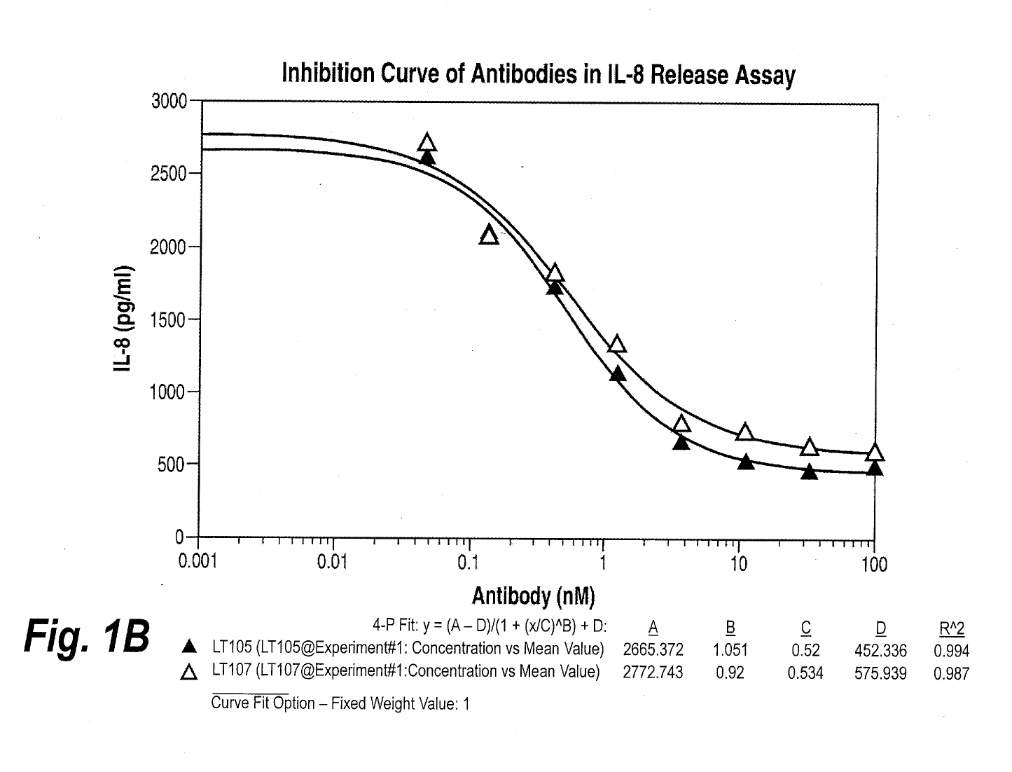

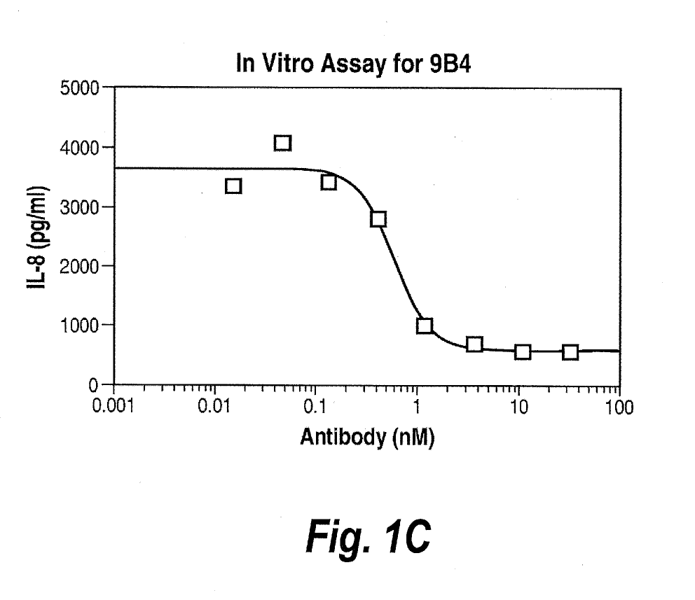

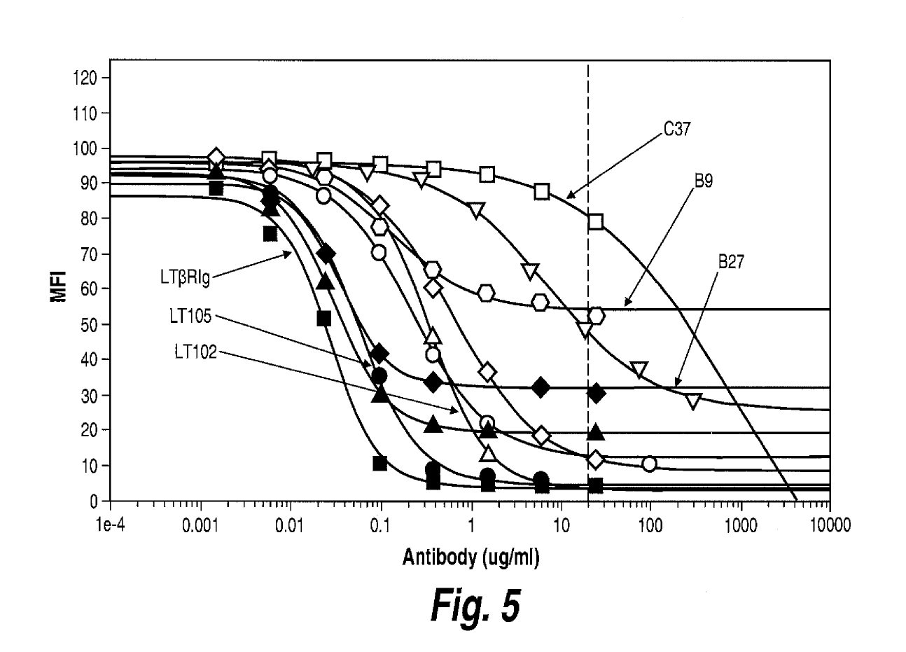

In one embodiment, the binding molecules of the invention inhibit LT binding to LTβR and/or an LT-induced biological activity at a lower concentration than the prior art antibodies. This can be easily seen when the concentration which inhibits an LT-induced biological activity (e.g., IL-8 release) by 50% (IC50) of antibodies comprising the LT binding sites of the invention is compared with antibodies comprising the prior art LT binding sites. The prior art antibodies require as much as 3 orders of magnitude more antibody to achieve 50% inhibition of LT binding to LTβR (see FIGS. 1, 4 and 5) and some do not achieve 50% inhibition at all. For these antibodies a “theoretical IC50” may be used for comparison. In calculating the IC50 values, the antibody concentration present during the pre-incubation step with antigen (LT) was used (rather than the final concentration of antibody after addition of cells and buffer).

In one embodiment, a binding molecule of the invention has an IC50 for inhibition of LTβR or LTβR-Ig binding or has an IC50 for inhibition of one or more LT biological activities of less than approximately 500 nM. In another embodiment, a binding molecule of the invention has an IC50 for inhibition of LTβR or LTβR-Ig binding or has an IC50 for inhibition of one or more LT biological activities of less than approximately 100 nM. In another embodiment, a binding molecule of the invention has an IC50 for inhibition of LTβR or LTβR-Ig binding or has an IC50 for inhibition of one or more LT biological activities of less than approximately 30 nM. In another embodiment, a binding molecule of the invention has an IC50 for inhibition of LTβR or LTβR-Ig binding or has an IC50 for inhibition of one or more LT biological activities of less than approximately 10 nM. In another embodiment, a binding molecule of the invention has an IC50 for inhibition of LTβR or LTβR-Ig binding or has an IC50 for inhibition of one or more LT biological activities of less than approximately 3 nM

In one embodiment, binding molecules of the invention have more than one of these improved properties, i.e., achieve greater than 70%, 80%, 90%, 95%, or 98% inhibition LTβR or LTβR-Ig binding or inhibition of one or more LT biological activities and an IC50 for inhibition of less than approximately 500 nM, 100 nM, 30 nM, 10 nM, or 3 nM.

In one embodiment, a binding molecule of the invention binds to LTα1β2 with an EC50 of less than approximately 0.3 nM. In another embodiment, a binding molecule of the invention binds to LTα1β2 with an EC50 of less than approximately 0.1 nM. In another embodiment, a binding molecule of the invention binds to LTα1β2 with an EC50 of less than approximately 0.03 nM.

In one embodiment, a binding molecule of the invention a binding molecule of the invention inhibits one or more LT biological activities (e.g., IL-8 release) by at least 90% with an IC50 of 100 nM or less. In one embodiment, a binding molecule of the invention a binding molecule of the invention inhibits one or more LT biological activities (e.g., IL-8 release) by at least 90% with an IC50 of 30 nM or less. In one embodiment, a binding molecule of the invention a binding molecule of the invention inhibits one or more LT biological activities (e.g., IL-8 release) by at least 90% with an IC50 of 10 nM or less. In one embodiment, a binding molecule of the invention a binding molecule of the invention inhibits one or more LT biological activities (e.g., IL-8 release) by at least 90% with an IC50 of 3 nM or less. In one embodiment, the subject a binding molecule of the invention also inhibits LTβR or LTβR-Ig binding by at least 70% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In one embodiment, the subject a binding molecule of the invention also inhibits LTβR or LTβR-Ig binding by at least 80% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In one embodiment, the subject a binding molecule of the invention also inhibits LTβR or LTβR-Ig binding by at least 90% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In one embodiment, the subject a binding molecule of the invention also inhibits LTβR or LTβR-Ig binding by at least 95% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%). In one embodiment, the subject a binding molecule of the invention also inhibits LTβR or LTβR-Ig binding by at least 100% (e.g., under conditions in which a reference antibody, B9, (Produced by the cell line B9.C9.1, deposited with the ATCC under Accession number HB11962 or a molecule comprising an antigen binding region thereof, inhibits the LT α1β2-induced biological activity in a cell by about 50%).

C. Binding to a Novel Region of LT

The binding molecules of the instant invention do not bind to LTα3 (or, as in the case of 103), if they do bind to LTα3, do not bind in such a way as to block the binding of LTα3 to TNFR. In addition, the binding molecules of the invention all block the binding of LT to LTβR or LTβR-Ig. In one embodiment, an anti-LT binding molecule of the invention competes for binding to LT with an anti-LT antibody of the invention. Accordingly, in certain embodiments, a binding moiety employed in the compositions of the invention may bind to the same epitope as a reference antibody in a competition assay, e.g., an AOD9, 108, 107, 105, 9B4, A1D5, 102, or 101/103 antibody described herein For example, a binding moiety may be derived from an antibody which cross-blocks (i.e., competes for binding with) an ant-LT antibody of the invention or otherwise interferes with the binding of the antibody.

A binding molecule is said to “competitively inhibit” or “competitively block” binding of the ligand if it specifically or preferentially binds to the epitope to the extent that binding of the ligand (e.g. LT) to LTβR or LTβR-Ig is inhibited or blocked (e.g. sterically blocked) in a manner that is dependent on the concentration of the ligand. For example, when measured biochemically, competitive inhibition at a given concentration of binding molecule can be overcome by increasing the concentration of ligand in which case the ligand will outcompete the binding molecule for binding to the target molecule (e.g., LTβR). Without being bound to any particular theory, competition is thought to occur when the epitope to which the binding molecule binds is located at or near the binding site of the ligand, thereby preventing binding of the ligand. Competitive inhibition may be determined by methods well known in the art and/or described in the Examples, including, for example, competition ELISA assays. In one embodiment, a binding molecule of the invention competitively inhibits binding of an anti-LT antibody selected from the group consisting of AOD9, 108, 107, 105, 9B4, A1D5, 102, or 101/103 to LT (or competes with one of the antibodies ability to reduce the binding of LT to LTβR or to downmodulate LT-mediated signaling) by at least 90%, at least 80%, or at least 70%.

In one embodiment, a binding molecule of the invention competitively inhibits binding of the AOD9 antibody to LT. In one embodiment, a binding molecule of the invention competitively inhibits binding of the 108 antibody to LT. In one embodiment, a binding molecule of the invention competitively inhibits binding of the 107 antibody to LT.

In one embodiment, a binding molecule of the invention competitively inhibits binding of the 105 or 9B4 antibody to LT. In one embodiment, a binding molecule of the invention competitively inhibits binding of the A1D5 antibody to LT. In one embodiment, a binding molecule of the invention competitively inhibits binding of the 102 antibody to LT. In one embodiment, a binding molecule of the invention competitively inhibits binding of the 101/103 antibody to LT.

Other antibodies which bind to a competitive epitope of LT may be identified using art-recognized methods and their variable regions characterized. Such antibodies may be used as binding molecules or their variable regions may be used as binding sites and incorporated into a binding molecule of the invention. For example, the CDRs of such antibodies may be incorporated into a binding molecule of the invention. For example, once antibodies to various fragments of, or to the full-length LT without the signal sequence, have been produced, determining which amino acids, or epitope, of LT to which the antibody or antigen binding fragment binds can be determined by epitope mapping protocols as known in the art (e.g. double antibody-sandwich ELISA as described in “Chapter 11—Immunology,” Current Protocols in Molecular Biology, Ed. Ausubel et al., v.2, John Wiley & Sons, Inc. (1996)). Additional epitope mapping protocols may be found in Morris, G. Epitope Mapping Protocols, New Jersey: Humana Press (1996), which are both incorporated herein by reference in their entireties. Epitope mapping can also be performed by commercially available means (i.e. ProtoPROBE, Inc. (Milwaukee, Wis.)).

In yet another embodiment, a binding molecule of the invention may comprise a binding site that binds to certain amino acid residues of LT or certain amino acids of LT may be critical for its binding. The amino acid positions in LT disclosed below refer to the position of the amino acid in the mature form of the protein. For the sequence of the mature LTβ protein, see Genbank entries GI:292277 and 4505035 and Browning J. et al., Cell 72:847-856 (1993), all of which are hereby incorporated by reference in their entirety.

In one embodiment, the invention pertains to an isolated binding molecule that specifically binds to an epitope of LT, wherein the binding to the LT epitope by the binding molecule is competitively blocked in a dose-dependent manner by the 102 antibody. In another embodiment, amino acids 193 (R) and 194 (R) of LTβ (as set forth in SEQ ID NO: 12, below) are critical for binding of the binding molecule. The sequence of LTβ is set forth below:

| | |
|---|---|
| 1 | MGALGLEGRG GRLQGRGSLL LAVAGATSLV TLLLAVPITV |
| | LAVLALVPQD |
| 51 | QGGLVTETAD PGAQAQQGLG FQKLPEEEPE TDLSPGLPAA |
| | HLIGAPLKGQ |
| 101 | GLGWETTKEQ AFLTSGTQFS DAEGLALPQD GLYYLYCLVG |
| | YRGRAPPGGG |
| 151 | DPQGRSVTLR SSLYRAGGAY GPGTPELLLE GAETVTPVLD |
| | PARRQGYGPL |
| 201 | WYTSVGFGGL VQLRRGERVY VNISHPDMVD FARGKTFFGA |
| | VMVG |

In one embodiment, the invention pertains to an isolated binding molecule that specifically binds to an epitope of LT, wherein the binding to the LT epitope by the binding molecule is competitively blocked in a dose-dependent manner by AOD9 antibody. In another embodiment, amino acids 151 (D) and 153 (Q) of LTβ (as set forth in SEQ ID NO: 12) are critical for binding of the binding molecule.

In one embodiment, the invention pertains to an isolated binding molecule that specifically binds to an epitope of LT, wherein the binding to the LT epitope by the binding molecule is competitively blocked in a dose-dependent manner by 101/103 antibody.

In one embodiment, the invention pertains to an isolated binding molecule that specifically binds to an epitope of LT, wherein the binding to the LT epitope by the binding molecule is competitively blocked in a dose-dependent manner by the 105 or the 9B4 antibody. In one embodiment, amino acids 96 (P), 97 (L), 98 (K) of LTβ are critical for binding of the binding molecule.

In one embodiment, the invention pertains to an isolated binding molecule that specifically binds to an epitope of LT, wherein the binding to the LT epitope by the binding molecule is competitively blocked in a dose-dependent manner by the 105 antibody. In one embodiment, amino acids 96 (P), 97 (L), 98 (K), 106 (T), 107 (T), and 108 (K) of LTβ (as set forth in SEQ ID NO:12) are critical for binding of the binding molecule.

In one embodiment, the invention pertains to an isolated binding molecule that specifically binds to an epitope of LT, wherein the binding to the LT epitope by the binding molecule is competitively blocked in a dose-dependent manner by A1D5 antibody. In one embodiment, amino acid 172 (P) (as set forth in SEQ ID NO: 12) of LTβ is critical for binding of the binding molecule.

In one embodiment, the invention pertains to an isolated binding molecule that specifically binds to an epitope of LT, wherein the binding to the LT epitope by the binding molecule is competitively blocked in a dose-dependent manner by the 107 antibody. In one embodiment, amino acids 151 (D) and 153 (Q) of LTβ (as set forth in SEQ ID NO: 12) are critical for binding of the binding molecule.

In one embodiment, the invention pertains to an isolated binding molecule that specifically binds to an epitope of LT, wherein the binding to the LT epitope by the binding molecule is competitively blocked in a dose-dependent manner by the 108 antibody.

D. Novel Structure

In yet another embodiment, an anti-LT binding molecules of the invention comprise an anti-LT binding site that shares certain structural features, e.g., amino acid sequence identity with an anti-LT binding site as described herein.

The CDR sequences of a panel of antibodies having the claimed functional activities are set fort in Tables 1 and 2 below.

In one embodiment, the invention pertains to a lymphotoxin (LT) binding molecule comprising a heavy chain variable region comprising heavy chain CDRs CDRH1, CDRH2 and CDRH3 and light chain variable region comprising light chain CDRs CDRL1, CDRL2, and CDRL3 wherein the light and heavy chain CDRs are derived from an antibody selected from the group consisting of AOD9, 108, 107, A1D5, 102, 101/103, 9B4, and 105.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the AOD9 antibody.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 108 antibody.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 107 antibody.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the A1D5 antibody.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 102 antibody.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 101/103 antibody.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 105 antibody.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein the CDRs are derived from the 9B4 antibody.

Analysis of the CDRs class of antibodies isolated according to the instant examples has facilitated the development of consensus CDR amino acid sequences. In one embodiment, a binding molecule of the invention comprises one or more consensus CDR sequences a described herein (see, e.g., Table 1 and 2). For example, embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein CDRH1 comprises the sequence GFSLX₁X₂Y/SGX₃H/G X₄X₅ (SEQ ID NO: 13), wherein X is any amino acid. In another embodiment, X₁ is selected from the group consisting of S or T; X₂ is selected from the group consisting of T, D, or N. In another embodiment, X₃ is selected from the group consisting of V, M or I, X₄ is absent or V, and X₅ is absent or S In one embodiment, 7/10 or 7/12 of the amino acids sequences of CDRH1 are identical to those in the consensus sequence. In one embodiment, the remaining 5 CDRs are derived from the A0D9 antibody, the 108 antibody, the 9B4 antibody, or the 107 antibody, or combinations thereof.

In another embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein CDRH1 comprises the sequence GX₁X₂X₃X₄X₅X₆X₇X₈X₉X₁₀ (SEQ ID NO: 14), and wherein X₁ is selected from the group consisting of Y or F; X₂ is selected from the group consisting of S, T, or V; X₃ is selected from the group consisting of F or I; X₄ is selected from the group consisting of T or S; X₅ is selected from the group consisting of G, D, or S; X₆ is selected from the group consisting of Y, S, or G; X₇ is selected from the group consisting of F, Y, or W; X₈ is selected from the group consisting of M or Y; X₉ is selected from the group consisting of N, Y or W; and X₁₀ is selected from the group consisting of absent or N. In one embodiment, the remaining 5 CDRs are derived from the A1D5, A105, 102 or the 101/103 antibody.

In another embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein CDRH2 comprises the sequence VIWX₁GGX₂TX₃X₄NAX₅FX₆S (SEQ ID NO: 2). In one embodiment, X is any amino acid. In another embodiment, X₁ is selected from the group consisting of R or S; X₂ is selected from the group consisting of N or S; X₃ is selected from the group consisting of N or D; X₄ is selected from the group consisting of Y or H; X₅ is selected from the group consisting of A or V; and X₆ is selected from the group consisting of M, T, or I. In one embodiment, the remaining 5 CDRs of the binding molecule are derived from the A0D9 antibody, the 108 antibody, or the 107 antibody.

In another embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein CDRH2 comprises the sequence X₁X₂X₃X₄X₅X₆X₇X₈X₉X₁₀YX₁₁X₁₂X₁₃X₁₄X₁₅X₁₆ (SEQ ID NO: 15), and wherein X₁ is selected from the group consisting of R, T, G, or absent; X₂ is selected from the group consisting of I, H, or Y; X₃ is selected from the group consisting of N, G, Y, or I; X₄ is selected from the group consisting of P, D, Y, or S; X₅ is selected from the group consisting of Y, W, or G; X₆ is selected from the group consisting of N, T, or D; X₇ is selected from the group consisting of G, D or S; X₈ is selected from the group consisting of D, Y, or S; X₉ is selected from the group consisting of S, T, K, or N; X₁₀ is selected from the group consisting of F, H, D, R, or N; X₁₁ is selected from the group consisting of N, P, or T; X₁₂ is selected from the group consisting of Q, D, G, or P; X₁₀ is selected from the group consisting of K or S; X₁₄ is selected from the group consisting of F, V, or L; X₁₅ is selected from the group consisting of K or Q; and X₁₆ is selected from the group consisting of D, G, or N. In one embodiment, the remaining 5 CDRs are derived from the A1D5, 102, the 9B4, 105 or the 101/103 antibodies or combinations thereof.

In one embodiment, the invention pertains to an LT binding molecule comprising a heavy chain variable region comprising heavy chain CDRs CDRH1, CDRH2 and CDRH3 and light chain variable region comprising light chain CDRs CDRL1, CDRL2, and CDRL3, wherein CDRH3 comprises the sequence G/AYYG/A (SEQ ID NO: 16). In one embodiment, the remaining 5 CDRs are derived from the A0D9, the 107, 108, the 9B4 antibodies or combinations thereof.

In one embodiment, the invention pertains to an LT binding molecule comprising a light chain variable region comprising heavy chain CDRs CDRH1, CDRH2 and CDRH3 and light chain variable region comprising light chain CDRs CDRL1, CDRL2, and CDRL3, wherein CDRL1 comprises the sequence or X₁ASQDX₂X₃X₄X₅LX₆ (SEQ ID NO: 4) wherein X is any amino acid. In one embodiment, X₁ is selected from the group consisting of K or R; X₂ is selected from the group consisting of I or M; X₃ is selected from the group consisting of N or S; X₄ is selected from the group consisting of T or N; X₅ is selected from the group consisting of Y or F; X₆ is selected from the group consisting of N, T, or R. In one embodiment, the remaining 5 CDRs are derived from the A0D9 antibody, the 108 antibody, the 107 antibody, the A1D5 antibody, or the 101/103 antibody.

In one embodiment, the invention pertains to an LT binding molecule comprising a light chain variable region comprising heavy chain CDRs CDRH1, CDRH2 and CDRH3 and light chain variable region comprising light chain CDRs CDRL1, CDRL2, and CDRL3, wherein CDRL1 comprises the sequence or RASX₁SV X₂X₃X₄X₅ (SEQ ID NO: 3) wherein X is any amino acid. In one embodiment, X₁ is selected from the group consisting of E or S; X₂ is selected from the group consisting of D or S; X₃ is selected from the group consisting of N or Y; X₄ is selected from the group consisting of Y or M; X₅ is selected from the group consisting of G or I. In one embodiment, the remaining 5 CDRs are derived from the 105 antibody or the 9B4 antibody or combinations thereof.

In one embodiment, the invention pertains to an LT binding molecule comprising a light chain variable region comprising heavy chain CDRs CDRH1, CDRH2 and CDRH3 and light chain variable region comprising light chain CDRs CDRL1, CDRL2, and CDRL3, wherein CDRL2 comprises the sequence RAX₁RLX₂D (SEQ ID NO: 5) wherein X is any amino acid. In one embodiment, X₁ is selected from the group consisting of N or D; X₂ is selected from the group consisting of V or L. In one embodiment, the remaining 5 CDRs are derived from the A0D9 antibody, the 108 antibody, the 107 antibody, or the 101/103 antibody, or combinations thereof.

In another embodiment, CDRL2 comprises the sequence X₁X₂SX₃X₄X₅S(SEQ ID NO: 17), wherein X₁ is selected from the group consisting of Y, R, A, or K; X₂ is selected from the group consisting of T, A, or V; X₃ is selected from the group consisting of K, S, or N; X₄ is selected from the group consisting of L or R; X₅ is selected from the group consisting of H, E, A, or F. In one embodiment, the remaining 5 CDRs are derived from the A1D5 antibody, the 102 antibody, the 105 antibody, the 105A antibody, the 105B antibody, the 105C antibody, or the 9B4 antibody.

In another embodiment, the invention is directed to an LT binding molecule comprising a light chain variable region comprising heavy chain CDRs CDRH1, CDRH2 and CDRH3 and light chain variable region comprising light chain CDRs CDRL1, CDRL2, and CDRL3, wherein CDRL3 comprises the sequence X₁QX₂X₃X₄X₅PX₆T (SEQ ID NO: 18), wherein X₁ is selected from the group consisting of Q or F; X₂ is selected from the group consisting of Y, V, G, W, or S; X₃ is selected from the group consisting of D, S, or N; X₄=D, H, Y, or K; X₅ is selected from the group consisting of F, N, or D; and X₆=W, L, or Y. In one embodiment, the remaining 5 CDRs are derived from the 108, 107, A1D5, 102, 9B4, or 105 antibodies or combinations thereof.

In another embodiment, CDRL3 comprises the sequence LX₁X₂DX₃FPX₄T (SEQ ID NO: 19), wherein X₁ is selected from the group consisting of H or Q; X₂ is selected from the group consisting of H or Y; X₃ is selected from the group consisting of A or K; X₄ is selected from the group consisting of W or P. In one embodiment, the remaining 5 CDRs are derived from the A0D9 or 101/103 antibodies or combinations thereof.

In another embodiment, the present invention provides an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 sequences of the antibodies described herein (e.g., Kabat CDRs or Chothia CDRs (exemplary sites for substitution are shown in Table 1), except for one, two, three, four, five, or six amino acid substitutions in any one VH-CDR. In larger CDRs, e.g., VH-CDR-3, additional substitutions may be made in the CDR, as long as the VH comprising the VH-CDR specifically or preferentially binds to LT. In certain embodiments the amino acid substitutions are conservative.

In another embodiment, the present invention provides an isolated polypeptide comprising, consisting essentially of, or consisting of an immunoglobulin light chain variable region (VL) in which the VL-CDR1, VL-CDR2 and VL-CDR3 regions have polypeptide sequences which are identical to the VL-CDR1, VL-CDR2 and VL-CDR3 sequences of the antibodies described herein (e.g., Kabat CDRs or Chothia CDRs (exemplary sites for substitution are shown in Table 2), except for one, two, three, four, five, or six amino acid substitutions in any one VL-CDR. In certain embodiments the amino acid substitutions are conservative.

In one embodiment, changes to the CDRs of a binding molecule can be made to obtain a binding molecule which has improved properties, e.g. binding properties or physicochemical properties, e.g., solubility. For example, in one embodiment, changes may be made to one or more CDRs of the heavy or light chain which affect self-association to improve the solubility of the molecule. In one embodiment, such changes result in substitution of an amino acid with a replacement amino acid provided for by the motifs set forth in Tables 1 and 2. In one embodiment, at least one change is made to CDRL2 (e.g., of the 105 antibody). In another embodiment, two changes are made to CDRL2 (e.g., of the 105 antibody).

For example, in one embodiment, a version of the light chain of the 105 antibody having a mutation in CDRL2 of R at Kabat position 54 to K (version A), a second version having a mutation in CDRL2 of N at Kabat position 57 to S (version B), as well as a third version having both mutations in CDRL2 (comprising the K at Kabat position 54 and the S at Kabat position 57; version C) may be made. As shown in the instant examples, antibodies comprising these modified versions of CDRL2 demonstrated improved solubility.

LT binding molecules of the binding molecules of the invention may comprise antigen recognition sites, entire variable regions, or one or more CDRs derived from one or more starting or parental anti-LT antibodies of the invention.

In one embodiment, given the homology among the A0D9, 108, 9B4, and 107 heavy chain CDRs, various combinations can be made. For example, in one embodiment, an A0D9 heavy chain CDRH1 may be substituted for a 108, 9B4, or 107 CDRH1 and combined with CDRH2 and CDRH3 from a any of these antibody variable regions.

In another embodiment, given the homology among the A0D9, 108, 9B4, 101/103, and 107 light chain CDRs, various combinations can be made. For example, in one embodiment, an A0D9 light chain CDRL11 may be substituted for a 108, 9B4, 101/103, or 107 CDRL1 and combined with CDRL2 and CDRL3 from any of these antibody variable regions.

In another embodiment, the heavy chain of a first anti-LT antibody of the invention can be combined with the light chain of a second anti-LT antibody of the invention. For example, given the homology among the A0D9, 108, and 107 heavy chain CDRs, an A0D9 heavy chain may be combined with a 108 or 107 light chain to generate an anti-LT binding site. In another embodiment, a 108 heavy chain may be combined with an A0D9 or 107 light chain to generate an anti-LT binding site. In yet another embodiment, a 108 heavy chain may be combined with a A0D9 or 107 light chain to generate an anti-LT binding site.

In yet another embodiment, various versions of anti-LT antibody light and heavy chains can be combined. For example, in one embodiment, various versions of the 105 antibody light and heavy chains described here can be combined. As set forth herein, many of these versions demonstrate improved solubility as compared with the starting 105 antibody. Exemplary combinations of 105 light and heavy chains include: H1/L0 (heavy chain version 1 and light chain version 0); H1/Lversion A; H1/Lversion B; H1/L10; H1/L12; H1/L13; H11/L10; H11/L12; H11/L13; H14/L10; and H14/L12.

The invention also pertains to polynucleotide sequences encoding the subject binding molecules.

In certain embodiments, the polynucleotide or nucleic acid molecule is a DNA or RNA molecule. In the case of DNA, a polynucleotide comprising a nucleic acid which encodes a polypeptide normally may include a promoter and/or other transcription or translation control elements operably associated with one or more coding regions. In an operable association a coding region for a gene product, e.g., a polypeptide, is associated with one or more regulatory sequences in such a way as to place expression of the gene product under the influence or control of the regulatory sequence(s).

Nucleic acid molecules encoding anti-LT binding sites may be operably linked to nucleotide sequences encoding one or more constant region moieties or to other desired nucleotide sequences that may or may not be derived from an antibody. DNA fragments (such as a polypeptide coding region and a promoter associated therewith) are “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is an RNA molecule, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase. In one embodiment, a binding molecule of the invention is the mature form of the molecule lacking the signal peptide.

Also, as described in more detail elsewhere herein, the present invention includes compositions comprising one or more of the polynucleotides described above.

### III. Exemplary Forms of Binding Molecules

A. Anti-LT Antibodies

In certain embodiments, LT binding molecules of the invention are antibodies. Given the data disclosed in the instant application, it is apparent that antibodies that bind to LT and are superior to those previously generated can be made. In one embodiment, the invention pertains to antibodies that are functionally related to those disclosed herein. In one embodiment, the invention pertains to antibodies that are structurally related to those disclosed herein. In another embodiment, the invention pertains to antibodies that are structurally and functionally related to those disclosed herein. Antibodies of the present invention can be produced by methods known in the art for the synthesis of antibodies, in particular, by chemical synthesis or preferably, by recombinant expression techniques as described herein. For example, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Yet other embodiments of the present invention comprise the generation of human or substantially human antibodies, e.g., in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

In certain embodiments both the variable and constant regions of LT antibodies, or antigen-binding fragments, variants, or derivatives thereof are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as described in more detail elsewhere herein.

Polyclonal antibodies to an epitope of interest can be produced by various procedures well known in the art. For example, an antigen comprising the epitope of interest can be administered to various host animals including, but not limited to, rabbits, mice, rats, chickens, hamsters, goats, donkeys, etc., to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal LT antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using LT knockout mice to increase the regions of epitope recognition. Monoclonal antibodies can be prepared using a wide variety of techniques known in the art including the use of hybridoma and recombinant and phage display technology as described elsewhere herein.

Using art recognized protocols, in one example, antibodies are raised in mammals by multiple subcutaneous or intraperitoneal injections of the relevant antigen (e.g., purified LTα1β2 or cells expressing or cellular extracts comprising LTα1β2) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen. In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Those skilled in the art will also appreciate that DNA encoding antibodies or antibody fragments (e.g., antigen binding sites) may also be derived from antibody libraries, such as phage display libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv OE DAB (individual Fv region from light or heavy chains) or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002) each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Yet another exemplary embodiment, high affinity human Fab libraries are designed by combining immunoglobulin sequences derived from human donors with synthetic diversity in selected complementarity determining regions such as CDR H1 and CDR H2 (see, e.g., Hoet et al., Nature Biotechnol., 23:344-348 (2005), which is incorporated herein by reference). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. For example, DNA sequences encoding VH and VL regions are amplified or otherwise isolated from animal cDNA libraries (e.g., human or murine cDNA libraries of lymphoid tissues) or synthetic cDNA libraries. In certain embodiments, the DNA encoding the VH and VL regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH or VL regions are usually recombinantly fused to either the phage gene III or gene VIII. Phage expressing an antigen binding domain that binds to an antigen of interest (i.e., an LT polypeptide or a fragment thereof) can be selected or identified with antigen, e.g., using labeled antigen or antigen bound or captured to a solid surface or bead.

Additional examples of phage display methods that can be used to make antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies.

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988). See also, U.S. Pat. No. 5,565,332.)

An “affinity-matured” antibody is an antibody with one or more alterations in one or more CDRs thereof that result in an improvement in the affinity of the antibody for antigen, compared to a parent antibody that does not possess those alteration(s). Preferred affinity matured antibodies will have nanomolar or even picomolar affinities for the target antigen. Affinity-matured antibodies are produced by procedures known in the art. Marks et al Bio/Technology 10:779-783 (1992) describes affinity maturation by VH and VL domain shuffling. Random mutagenesis of CDR and/or framework residues is described by: Barbas et al, Proc Nat. Acad. Sci, USA 91:3809-3813 (1994); Schier et al., Gene 169:147-155 (1995); Yelton et al, J. Immunol. 155:1994-2004 (1995); Jackson et al, J. Immunol. 154.7):3310-9 (1995); and Hawkins et al, J. MoI Biol. 226:889-896 (1992).

B. Single Chain Binding Molecules

In other embodiments, a binding molecule of the invention may be a single chain binding molecule (e.g., a singe chain variable region or scFv). Techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain binding molecules. Single chain antibodies are formed by linking the heavy and light chain fragments of the Fv region via an amino acid bridge, resulting in a single chain antibody. Techniques for the assembly of functional Fv fragments in E coli may also be used (Skerra et al., Science 242:1038-1041 (1988)).

In certain embodiments, binding molecules of the invention are scFv molecules (e.g., a VH and a VL domain from an anti-LT antibody of the invention joined by an scFv linker) or comprise such molecules. scFv molecules may be conventional scFv molecules or stabilized scFv molecules. Stabilized scFvs comprising stabilizing mutations, disulfide bonds, or optimized linkers which confer improved stability (e.g., improved thermal stability) to the scFv or to a binding molecule comprising the scFv are described in detail in U.S. patent application Ser. No. 11/725,970, which is incorporated by reference herein in its entirety.

In other embodiments, binding molecules of the invention are polypeptides comprising scFv molecules. In certain embodiments, a multispecific binding molecule may be created by linking a scFv molecule (e.g., a stabilized scFv molecule) with an anti-LT antibody described supra, or a monospecific binding molecule comprising the binding site of one of the anti-LT antibodies, wherein the scFv molecule and the parent binding molecule have the same binding specificity. In one embodiment, a binding molecule of the invention is a naturally occurring anti-LT antibody to which an scFv molecule has been fused.

Stabilized scFv molecules have improved thermal stability (e.g., melting temperature (Tm) values greater than 54° C. (e.g. 55, 56, 57, 58, 59, 60° C. or greater) or T50 values greater than 39° C. (e.g. 40, 41, 42, 43, 44, 45, 46, 47, 48, 50, 51, 52, 53, 54, 55, 56, 57, 58, or 59° C.). The stability of scFv molecules of the invention or fusion proteins comprising them can be evaluated in reference to the biophysical properties (e.g., thermal stability) of a conventional (non-stabilized) scFv molecule or a binding molecule comprising a conventional scFv molecule. In one embodiment, the binding molecules of the invention have a thermal stability that is greater than about 0.1, about 0.25, about 0.5, about 0.75, about 1, about 1.25, about 1.5, about 1.75, about 2, about 3, about 4, about 5, about 6, about 7, about 8, about 9, or about 10 degrees Celsius than a control binding molecule (eg. a conventional scFv molecule).

In one embodiment, the scFv linker consists of the amino acid sequence (Gly₄Ser)₄ or comprises a (Gly₄Ser)₄ sequence. Other exemplary linkers comprise or consist of (Gly₄Ser)₃ and (Gly₄Ser)₅ sequences. scFv linkers of the invention can be of varying lengths. In one embodiment, an scFv linker of the invention is from about 5 to about 50 amino acids in length. In another embodiment, an scFv linker of the invention is from about 10 to about 40 amino acids in length. In another embodiment, an scFv linker of the invention is from about 15 to about 30 amino acids in length. In another embodiment, an scFv linker of the invention is from about 17 to about 28 amino acids in length. In another embodiment, an scFv linker of the invention is from about 19 to about 26 amino acids in length. In another embodiment, an scFv linker of the invention is from about 21 to about 24 amino acids in length.

In certain embodiments, the stabilized scFv molecules of the invention comprise at least one disulfide bond which links an amino acid in the VL domain with an amino acid in the VH domain. Cysteine residues are necessary to provide disulfide bonds. Disulfide bonds can be included in an scFv molecule of the invention, e.g., to connect FR4 of VL and FR2 of VH or to connect FR2 of VL and FR4 of VH. Exemplary positions for disulfide bonding include: 43, 44, 45, 46, 47, 103, 104, 105, and 106 of VH and 42, 43, 44, 45, 46, 98, 99, 100, and 101 of VL, Kabat numbering. Exemplary combinations of amino acid positions which are mutated to cysteine residues include: VH44-VL100, VH105-VL43, VH105-VL42, VH44-VL101, VH106-VL43, VH104-VL43, VH44-VL99, VH45-VL98, VH46-VL98, VH103-VL43, VH103-VL44, and VH103-VL45. In one embodiment, a disulfide bond links V_{H} amino acid 44 and V_{L} amino acid 100.

In one embodiment, a stabilized scFv molecule of the invention comprises an scFv linker having the amino acid sequence (Gly₄ Ser)₄ interposed between a V_{H} domain and a V_{L} domain, wherein the V_{H} and V_{L} domains are linked by a disulfide bond between an amino acid in the V_{H} at amino acid position 44 and an amino acid in the V_{L} at amino acid position 100.

In other embodiments the stabilized scFv molecules of the invention comprise one or more (e.g. 2, 3, 4, 5, or more) stabilizing mutations within a variable domain (VH or VL) of the scFv. In one embodiment, the stabilizing mutation is selected from the group consisting of: a) substitution of an amino acid (e.g., glutamine) at Kabat position 3 of VL, e.g., with an alanine, a serine, a valine, an aspartic acid, or a glycine; (b) substitution of an amino acid (e.g., serine) at Kabat position 46 of VL, e.g., with leucine; (c) substitution of an amino acid (e.g., serine) at Kabat position 49 of VL, e.g., with tyrosine or serine; (d) substitution of an amino acid (e.g., serine or valine) at Kabat position 50 of VL, e.g., with serine, threonine, and arginine, aspartic acid, glycine, or lysine; (e) substitution of amino acids (e.g., serine) at Kabat position 49 and (e.g., serine) at Kabat position 50 of VL, respectively with tyrosine and serine; tyrosine and threonine; tyrosine and arginine; tyrosine and glycine; serine and arginine; or serine and lysine; (f) substitution of an amino acid (e.g., valine) at Kabat position 75 of VL, e.g., with isoleucine; (g) substitution of an amino acid (e.g., proline) at Kabat position 80 of VL, e.g., with serine or glycine; (h) substitution of an amino acid (e.g., phenylalanine) at Kabat position 83 of VL, e.g., with serine, alanine, glycine, or threonine; (i) substitution of an amino acid (e.g., glutamic acid) at Kabat position 6 of VH, e.g., with glutamine; (j) substitution of an amino acid (e.g., lysine) at Kabat position 13 of VH, e.g., with glutamate; (k) substitution of an amino acid (e.g., serine) at Kabat position 16 of VH, e.g., with glutamate or glutamine; (l) substitution of an amino acid (e.g., valine) at Kabat position 20 of VH, e.g., with an isoleucine; (m) substitution of an amino acid (e.g., asparagine) at Kabat position 32 of VH, e.g., with serine; (n) substitution of an amino acid (e.g., glutamine) at Kabat position 43 of VH, e.g., with lysine or arginine; (o) substitution of an amino acid (e.g., methionine) at Kabat position 48 of VH, e.g., with an isoleucine or a glycine; (p) substitution of an amino acid (e.g., serine) at Kabat position 49 of VH, e.g., with glycine or alanine; (q) substitution of an amino acid (e.g., valine) at Kabat position 55 of VH, e.g., with a glycine; (r) substitution of an amino acid (e.g., valine) at Kabat position 67 of VH, e.g., with an isoleucine or a leucine; (s) substitution of an amino acid (e.g., glutamic acid) at Kabat position 72 of VH, e.g., with aspartate or asparagine; (t) substitution of an amino acid (e.g., phenylalanine) at Kabat position 79 of VH, e.g., with serine, valine, or tyrosine; and (u) substitution of an amino acid (e.g., proline) at Kabat position 101 of VH, e.g., with an aspartic acid.

C. Single Domain Binding Molecules

In certain embodiments, the binding molecule is or comprises a single domain binding molecule (e.g. a single domain antibody), also known as nanobodies. Exemplary single domain molecules include an isolated heavy chain variable domain (V_{H}) of an antibody, i.e., a heavy chain variable domain, without a light chain variable domain, and an isolated light chain variable domain (V_{L}) of an antibody, i.e., a light chain variable domain, without a heavy chain variable domain. Exemplary single-domain antibodies employed in the binding molecules of the invention include, for example, the Camelid heavy chain variable domain (about 118 to 136 amino acid residues) as described in Hamers-Casterman, et al., Nature 363:446-448 (1993), and Dumoulin, et al., Protein Science 11:500-515 (2002). Multimers of single-domain antibodies are also within the scope of the invention. Other single domain antibodies include shark antibodies (e.g., shark Ig-NARs). Shark Ig-NARs comprise a homodimer of one variable domain (V-NAR) and five C-like constant domains (C-NAR), wherein diversity is concentrated in an elongated CDR3 region varying from 5 to 23 residues in length In camelid species (e.g., llamas), the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH. Methods for making single domain binding molecules are described in U.S. Pat. Nos. 6,005,079 and 6,765,087, both of which are incorporated herein by reference.

D. Minibodies

In certain embodiments, the binding molecules of the invention are minibodies or comprise minibodies. Minibodies can be made using methods described in the art (see e.g., U.S. Pat. No. 5,837,821 or WO 94/09817A1). In certain embodiments, a minibody is a binding molecule that comprises only 2 complementarity determining regions (CDRs) of a naturally or non-naturally (e.g., mutagenized) occurring heavy chain variable domain or light chain variable domain, or combination thereof. An example of such a minibody is described by Pessi et al., Nature 362:367-369 (1993). Another exemplary minibody comprises a scFv molecule that is linked or fused to a CH3 domain or a complete Fc region. Multimers of minibodies are also within the scope of the invention.

E. Binding Molecule Fragments

Unless it is specifically noted, as used herein a “fragment” in reference to a binding molecule refers to an antigen-binding fragment, i.e., a portion of the binding which specifically binds to the antigen. In one embodiment, a binding molecule of the invention is an antibody fragment or comprises such a fragment. Antibody fragments that recognize specific epitopes may be generated by known techniques. For example, Fab and F(ab′)₂ fragments may be produced recombinantly or by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

F. Multivalent Minibodies

In one embodiment, the multispecific binding molecules of the invention are multivalent minibodies having at least one scFv fragment with a first binding site and at least one scFv with a second binding site. The binding sites of the two scFv molecules may be the same or different. In preferred embodiments, at least one of the scFv molecules is stabilized. An exemplary bispecific bivalent minibody construct comprises a CH3 domain fused at its N-terminus to a connecting peptide which is fused at its N-terminus to a VH domain which is fused via its N-terminus to a (Gly4Ser)n flexible linker which is fused at its N-terminus to a VL domain. In certain embodiments, multivalent minibodies may be biavalent, trivalent (e.g., triabodies), bispecific (e.g., diabodies), or tetravalent (e.g., tetrabodies).

In another embodiment, the binding molecules of the invention are scFv tetravalent minibodies, with each heavy chain portion of the scFv tetravalent minibody containing first and second scFv fragments having different binding specificities. In preferred embodiments at least one of the scFv molecules is stabilized. Said second scFv fragment may be linked to the N-terminus of the first scFv fragment (e.g. bispecific N_{H} scFv tetravalent minibodies or bispecific N_{L} scFv tetravalent minibodies). Alternatively, the second scFv fragment may be linked to the C-terminus of said heavy chain portion containing said first scFv fragment (e.g. bispecific C-scFv tetravalent minibodies). Where the first and second scFv fragments of a first heavy chain portion of a bispecific tetravalent minibody bind the same target LT molecule, at least one of the first and second scFv fragments of the second heavy chain portion of the bispecific tetravalent minibody may bind the same or different LT target molecule.

G. Multispecific Antibodies

Multispecific binding molecules of the invention may comprise at least two binding sites, wherein at least one of the binding sites is derived from or comprises a binding site from one of the monospecific binding molecules described supra. In certain embodiments, at least one binding site of a multispecific binding molecule of the invention is an antigen binding region of an antibody or an antigen binding fragment thereof (e.g. an antibody or antigen binding fragment described supra).

In certain embodiments, a multispecific binding molecule of the invention is bispecific. Bispecific binding molecules may be bivalent or of a higher valency (e.g., trivalent, tetravalent, hexavalent, and the like). Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

H. scFv-Containing Multispecific Binding Molecules

In one embodiment, the multispecific binding molecules of the invention are multispecific binding molecules comprising at least one scFv molecule, e.g. an scFv molecule described supra. In other embodiments, the multispecific binding molecules of the invention comprise two scFv molecules, e.g. a bispecific scFv (Bis-scFv). In certain embodiments, the scFv molecule is a conventional scFv molecule. In other embodiments, the scFv molecule is a stabilized scFv molecule described supra. In certain embodiments, a multispecific binding molecule may be created by linking a scFv molecule (e.g., a stabilized scFv molecule) with an anti-LT antibody described supra, or a monospecific binding molecule comprising the binding site of one of the anti-LT antibodies, wherein the scFv molecule and the parent binding molecule bind to different regions of LT/have different critical LT contact residues. In one embodiment, a binding molecule of the invention is a naturally occurring anti-LT antibody to which an scFv molecule has been fused. In one embodiment, such an scFv molecule is stabilized.

When a stabilized scFv is linked to a parent binding molecule, linkage of the stabilized scFv molecule preferably improves the thermal stability of the binding molecule by at least about 2° C. or 3° C. In one embodiment, the scFv-containing binding molecule of the invention has a 1° C. improved thermal stability as compared to a conventional binding molecule. In another embodiment, a binding molecule of the invention has a 2° C. improved thermal stability as compared to a conventional binding molecule. In another embodiment, a binding molecule of the invention has a 4, 5, 6° C. improved thermal stability as compared to a conventional binding molecule.

In one embodiment, the binding molecules of the invention are stabilized “antibody” or “immunoglobulin” molecules, e.g., naturally occurring antibody or immunoglobulin molecules (or an antigen binding fragment thereof) or genetically engineered antibody molecules that bind antigen in a manner similar to antibody molecules and that comprise an scFv molecule of the invention. As used herein, the term “immunoglobulin” includes a polypeptide having a combination of two heavy and two light chains whether or not it possesses any relevant specific immunoreactivity.

In one embodiment, the multispecific binding molecules of the invention comprise at least one scFv (e.g. 2, 3, or 4 scFvs, e.g., stabilized scFvs) linked to the C-terminus of an antibody heavy chain, wherein the scFv and antibody have different binding specificities. In another embodiment, the multispecific binding molecules of the invention comprise at least one scFv (e.g. 2, 3, or 4 scFvs, e.g., stabilized scFvs) linked to the N-terminus of an antibody heavy chain, wherein the scFv and antibody have different binding specificities. In another embodiment, the multispecific binding molecules of the invention comprise at least one scFv (e.g. 2, 3, or 4 scFvs or stabilized scFvs) linked to the N-terminus of an antibody light chain, wherein the scFv and antibody have different binding specificities. In another embodiment, the multispecific binding molecules of the invention comprise at least one scFv (e.g., 2, 3, or 4 scFvs or stabilized scFvs) linked to the N-terminus of the antibody heavy chain or light chain and at least one scFv (e.g., 2, 3, or 4 scFvs or stabilized scFvs) linked to the C-terminus of the heavy chain, wherein the scFvs have different binding specificity.

I. Multispecific Diabodies

In other embodiments, the binding molecules of the invention are multispecific diabodies. In one embodiment, the multispecific binding molecules of the invention are bispecific diabodies, with each arm of the diabody comprising tandem scFv fragments. In preferred embodiments, at least one of the scFv fragments is stabilized. In one embodiment, a bispecific diabody may comprise a first arm with a first binding specificity and a second arm with a second binding specificity. In another embodiment, each arm of the diabody may comprise a first scFv fragment with a first binding specificity and a second scFv fragment with a second binding specificity. In certain embodiments, a multispecific diabody can be directly fused to a complete Fc region or an Fc portion (e.g. a CH3 domain).

J. Multispecific Binding Molecule Fragments

In certain embodiments, binding molecule fragments of the invention may be made to be multispecific. Multispecific binding molecules of the invention include bispecific Fab2 or multispecific (e.g. trispecific) Fab3 molecules. For example, a multispecific binding molecule fragment may comprise chemically conjugated multimers (e.g. dimers, trimers, or tetramers) of Fab or scFv molecules having different specificities.

K. scFv2 Tetravalent Antibodies

In other embodiments, the multispecific binding molecules of the invention are scFv2 tetravalent antibodies with each heavy chain portion of the scFv2 tetravalent antibody containing an scFv molecule. In preferred embodiments, at least one of the scFv molecules are stabilized. The scFv fragments may be linked to the N-termini of a variable region of the heavy chain portions (e.g. N_{H} scFv2 tetravalent antibodies or N_{L} scFv2 tetravalent antibodies). Alternatively, the scFv fragments may be linked to the C-termini of the heavy chain portions of the scFv2 tetravalent antibody. Each heavy chain portion of the scFv2 tetravalent antibody may have variable regions and scFv fragments that bind the same or different target LT molecule or epitope. In the case of a multispecific molecule, where the scFv fragment and variable region of a first heavy chain portion of a scFc2 tetravalent antibody bind the same target molecule or epitope, at least one of the first and second scFv fragments of the second heavy chain portion of the bispecific tetravalent minibody binds a different target molecule or epitope.

L. Tandem Variable Domain Binding Molecules

In other embodiments, the multispecific binding molecule of the invention may comprise a binding molecule comprising tandem antigen binding sites. For example, a variable domain may comprise an antibody heavy chain that is engineered to include at least two (e.g., two, three, four, or more) variable heavy domains (VH domains) that are directly fused or linked in series, and an antibody light chain that is engineered to include at least two (e.g., two, three, four, or more) variable light domains (VL domains) that are direct fused or linked in series. The VH domains interact with corresponding VL domains to form a series of antigen binding sites wherein at least two of the binding sites bind the same, or different epitopes of LT. Tandem variable domain binding molecules may comprise two or more of heavy or light chains and are of higher order valency (e.g., bivalent or tetravalent). Methods for making tandem variable domain binding molecules are known in the art, see e.g. WO 2007/024715.

M. Multispecific Fusion Proteins

In another embodiment, a multispecific binding molecule of the invention is a multispecific fusion protein. As used herein the phrase “multispecific fusion protein” designates fusion proteins (as hereinabove defined) having at least two binding specificities described supra. Multispecific fusion proteins can be assembled, e.g., as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Preferred multispecific fusion proteins are bispecific. In certain embodiments, at least of the binding specificities of the multispecific fusion protein comprises an scFv, e.g., a stabilized scFv.

A variety of other multivalent antibody constructs may be developed by one of skill in the art using routine recombinant DNA techniques, for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al., U.S. Pat. No. 4,816,567; Morrison et al., Proc. Natl. Acad. Sci. U.S.A., 81, pp. 6851-55 (1984)).

Other methods which may be used to prepare multivalent antibody constructs are described in the following publications: Ghetie, Maria-Ana et al. (2001) Blood 97:1392-1398; Wolff, Edith A. et al. (1993) Cancer Research 53:2560-2565; Ghetie, Maria-Ana et al. (1997) Proc. Natl. Acad. Sci. 94:7509-7514; Kim, J. C. et al. (2002) Int. J. Cancer 97(4):542-547; Todorovska, Aneta et al. (2001) Journal of Immunological Methods 248:47-66; Coloma M. J. et al. (1997) Nature Biotechnology 15:159-163; Zuo, Zhuang et al. (2000) Protein Engineering (Suppl.) 13(5):361-367; Santos A. D., et al. (1999) Clinical Cancer Research 5:3118s-3123s; Presta, Leonard G. (2002) Current Pharmaceutical Biotechnology 3:237-256; van Spriel, Annemiek et al., (2000) Review Immunology Today 21(8) 391-397.

### IV. Modified Binding Molecules

In certain embodiments, at least one of the binding molecules of the invention may comprise one or more modifications. Modified forms of LT binding molecules of the invention can be made from whole precursor or parent antibodies using techniques known in the art.

In certain embodiments, modified LT binding molecules of the present invention are polypeptides which have been altered so as to exhibit features not found on the native polypeptide (e.g., a modification which results in reduction of function or enhancement of function, e.g., effector function). In one embodiment, one or more residues of the binding molecule may be chemically derivatized by reaction of a functional side group. In one embodiment, a binding molecule may be modified to include one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

In one embodiment, an LT binding molecule of the invention comprises a synthetic constant region wherein one or more domains are partially or entirely deleted (“domain-deleted binding molecules”). In certain embodiments compatible modified binding molecules will comprise domain deleted constructs or variants wherein the entire CH2 domain has been removed (ΔCH2 constructs). For other embodiments a short connecting peptide may be substituted for the deleted domain to provide flexibility and freedom of movement for the variable region. Those skilled in the art will appreciate that such constructs are particularly preferred due to the regulatory properties of the CH2 domain on the catabolic rate of the antibody. Domain deleted constructs can be derived using a vector encoding an IgG₁ human constant domain (see, e.g., WO 02/060955A2 and WO02/096948A2). This vector is engineered to delete the CH2 domain and provide a synthetic vector expressing a domain deleted IgG1 constant region.

In one embodiment, an LT binding molecule of the invention comprises an immunoglobulin heavy chain having deletion or substitution of a few or even a single amino acid as long as it permits association between the monomeric subunits. For example, in certain situations, the mutation of a single amino acid in selected areas of the CH2 domain may be enough to substantially reduce Fc binding. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the binding molecule may be altered through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified binding molecule. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides binding molecule that comprise, consist essentially of, or consist of, variants (including derivatives) of binding moieties (e.g., the VH regions and/or VL regions of an antibody molecule) described herein, which binding moieties immunospecifically bind to an LT polypeptide. Standard techniques known to those of skill in the art can be used to introduce mutations in the nucleotide sequence encoding an LT binding molecule, include, but are not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VH-CDR1, VH-CDR2, VH-CDR3, VL region, VL-CDR1, VL-CDR2, or VL-CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an LT polypeptide).

For example, it is possible to introduce mutations only in framework regions or only in CDR regions of a binding molecule of the invention (e.g., an antibody molecule). Introduced mutations may be silent or neutral missense mutations, i.e., have no, or little, effect on the ability to bind antigen, indeed some such mutations do not alter the amino acid sequence whatsoever. These types of mutations may be useful to optimize codon usage, or improve a hybridoma's antibody production. Alternatively, non-neutral missense mutations may alter a binding molecule's ability to bind antigen. For example, in an antibody the location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of an LT polypeptide) can be determined using techniques described herein or by routinely modifying techniques known in the art.

A. Covalent Attachment

LT binding molecules of the invention may be modified, e.g., by the covalent attachment of a molecule to the binding molecule such that covalent attachment does not prevent the binding molecule from specifically binding to its cognate epitope. For example, but not by way of limitation, the binding molecules of the invention may be modified (either to include or remove) glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

As discussed in more detail elsewhere herein, binding molecules of the invention may further be recombinantly fused to a heterologous polypeptide at the N- or C-terminus or chemically conjugated (including covalent and non-covalent conjugations) to polypeptides or other compositions. For example, LT-specific binding molecules may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

An LT binding molecule of the invention can be composed of amino acids joined to each other by peptide bonds or modified peptide bonds, i.e., peptide isosteres, and may contain amino acids other than the 20 gene-encoded amino acids. LT-specific binding molecules may be modified by natural processes, such as posttranslational processing, or by chemical modification techniques which are well known in the art. Such modifications are well described in basic texts and in more detailed monographs, as well as in a voluminous research literature. Modifications can occur anywhere in the LT-specific binding molecule, including the peptide backbone, the amino acid side-chains and the amino or carboxyl termini, or on moieties such as carbohydrates. It will be appreciated that the same type of modification may be present in the same or varying degrees at several sites in a given LT-specific binding molecule. Also, a given LT-specific binding molecule may contain many types of modifications. LT-specific binding molecule may be branched, for example, as a result of ubiquitination, and they may be cyclic, with or without branching. Cyclic, branched, and branched cyclic LT-specific binding molecule may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W.H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising an LT binding molecule, and a heterologous polypeptide. The heterologous polypeptide to which the antibody is fused may provide a desired functionality or may be useful to target LT polypeptide expressing cells. In one embodiment, a fusion protein of the invention comprises, consists essentially of, or consists of, a polypeptide having the amino acid sequence of any one or more of the binding sites of a binding molecule of the invention and a heterologous polypeptide sequence. In another embodiment, a fusion protein for use in the diagnostic and treatment methods disclosed herein comprises, consists essentially of, or consists of a polypeptide having the amino acid sequence of any one, two, or three of the VH-CDRs of an LT-specific binding molecule, or the amino acid sequence of any one, two, or three of the VL-CDRs of an LT-specific binding molecule, and a heterologous polypeptide sequence. In one embodiment, the fusion protein comprises a polypeptide having the amino acid sequence of a VH-CDR3 of an LT-specific binding molecule of the present invention, and a heterologous polypeptide sequence, which fusion protein specifically binds to at least one epitope of LT. In another embodiment, a fusion protein comprises a polypeptide having the amino acid sequence of at least one VH region of an LT-specific binding molecule of the invention and the amino acid sequence of at least one VL region of an LT-specific binding molecule of the invention and a heterologous polypeptide sequence. In one embodiment, the VH and VL regions of the fusion protein correspond to a single source binding molecule which specifically binds at least one epitope of LT. In yet another embodiment, a fusion protein for use in the diagnostic and treatment methods disclosed herein comprises a polypeptide having the amino acid sequence of any one, two, or three or more of the VH CDRs of an LT-specific binding molecule and the amino acid sequence of any one, two, or three or more of the VL CDRs of an LT-specific binding molecule, and a heterologous polypeptide sequence. In one embodiment, two, three, four, five, or six, of the VH-CDR(s) or VL-CDR(s) correspond to single source binding molecule of the invention. Nucleic acid molecules encoding these fusion proteins are also encompassed by the invention.

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

As discussed elsewhere herein, LT antibodies, or antigen-binding fragments, variants, or derivatives thereof of the invention may be fused to heterologous polypeptides to increase the in vivo half life of the polypeptides or for use in immunoassays using methods known in the art. For example, in one embodiment, PEG can be conjugated to the LT binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, LT binding molecules of the invention can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see for example U.S. Pat. Nos. 5,116,964 and 5,225,538). The precise site at which the fusion is made may be selected empirically to optimize the secretion or binding characteristics of the fusion protein. DNA encoding the fusion protein is then transfected into a host cell for expression.

LT binding molecules of the present invention may be used in non-conjugated form or may be conjugated to at least one of a variety of molecules, e.g., to improve the therapeutic properties of the molecule, to facilitate target detection, or for imaging or therapy of the patient. LT binding molecules of the invention can be labeled or conjugated either before or after purification, when purification is performed.

In particular, LT binding molecules of the invention may be conjugated to therapeutic agents, prodrugs, peptides, proteins, enzymes, viruses, lipids, biological response modifiers, pharmaceutical agents, or PEG.

Those skilled in the art will appreciate that conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the LT binding molecules of the invention are prepared in an analogous manner.

The present invention further encompasses LT binding molecules of the invention conjugated to a diagnostic or therapeutic agent. The LT binding molecules can be used diagnostically to, for example, monitor the development or progression of a disease as part of a clinical testing procedure to, e.g., determine the efficacy of a given treatment and/or prevention regimen. Detection can be facilitated by coupling the LT binding molecule to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

An LT binding molecule also can be detectably labeled by coupling it to a chemiluminescent compound. The presence of the chemiluminescent-tagged LT binding molecules is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an LT binding molecule can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the LT binding molecule will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the LT binding molecule, it is possible to detect the binding molecule through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

An LT binding molecule can also be detectably labeled using fluorescence emitting metals such as 152Eu, or others of the lanthanide series. These metals can be attached to the binding molecules using such metal chelating groups as diethylenetriaminepentacetic acid (DTPA) or ethylenediaminetetraacetic acid (EDTA).

Techniques for conjugating various moieties to binding molecules are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

In particular, binding molecules for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG. In another embodiment, a binding molecule for use in the diagnostic and treatment methods disclosed herein can be conjugated to a molecule that decreases vascularization of tumors. In other embodiments, the disclosed compositions may comprise binding molecules coupled to drugs or prodrugs. Still other embodiments of the present invention comprise the use of binding molecules conjugated to specific biotoxins or their cytotoxic fragments such as ricin, gelonin, Pseudomonas exotoxin or diphtheria toxin. The selection of which conjugated or unconjugated binding molecule to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, binding molecules may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99 labeled polypeptides may be prepared by ligand exchange processes, by reducing pertechnate (TcO₄⁻) with stannous ion solution, chelating the reduced technetium onto a Sephadex column and applying the binding polypeptides to this column, or by batch labeling techniques, e.g. by incubating pertechnate, a reducing agent such as SnCl₂, a buffer solution such as a sodium-potassium phthalate-solution, and the binding molecules. In any event, preferred radionuclides for directly labeling polypeptides are well known in the art and a particularly preferred radionuclide for direct labeling is ¹³¹I covalently attached via tyrosine residues. Binding molecules for use in the methods disclosed herein may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Additional preferred agents for conjugation to binding molecules, e.g., binding polypeptides are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Techniques for conjugating various moieties to a binding molecule are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

B. Reducing Immunogenicity

In certain embodiments, LT binding molecules of the invention or portions thereof are modified to reduce their immunogenicity using art-recognized techniques. For example, binding molecules or portions thereof can be humanized, primatized, or deimmunized. In one embodiment, chimeric binding molecules can be made or binding molecules may comprise at least a portion of a chimeric antibody molecule. In such case a non-human LT binding molecule, typically a murine or primate binding molecule, that retains or substantially retains the antigen-binding properties of the parent binding molecule, but which is less immunogenic in humans is constructed. This may be achieved by various methods, including (a) grafting the entire non-human variable domains onto human constant regions to generate chimeric binding molecule; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

In one embodiment, a binding molecule (e.g., an antibody) of the invention or portion thereof may be chimeric. A chimeric binding molecule is a binding molecule in which different portions of the binding molecule are derived from different animal species, such as antibodies having a variable region derived from a murine monoclonal antibody and a human immunoglobulin constant region. Methods for producing chimeric binding molecules are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) may be employed for the synthesis of said molecules. For example, a genetic sequence encoding a binding specificity of a mouse LT antibody molecule may be fused together with a sequence from a human antibody molecule of appropriate biological activity. As used herein, a chimeric binding molecule is a molecule in which different portions are derived from different animal species, such as those having a variable region derived from a murine monoclonal antibody and a human immunoglobulin constant region, e.g., humanized antibodies.

In another embodiment, a binding molecule of the invention or portion thereof is primatized. Methods for primatizing antibodies are disclosed by Newman, Biotechnology 10: 1455-1460 (1992). Specifically, this technique results in the generation of antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, a binding molecule (e.g., an antibody) of the invention or portion thereof is humanized. Humanized binding molecules are binding molecules having a binding specificity from a non-human species, i.e., having one or more complementarity determining regions (CDRs) from the non-human species antibody, and framework regions from a human immunoglobulin molecule. Often, framework residues in the human framework regions will be mutated, e.g., substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Other references for humanization of antibodies include: Kabat, E. A., Wu, T. T., Perry, H. M., Gottesman, K. S, and Foeller, C. (1991) Sequences of Proteins of Immunological Interest. 5^{th} Edition, U.S. Dept. Health and Human Services. U.S. Govt. Printing Office. Chothia, C., Lesk, A. M., Tramontano, A., Levitt, M., Smith-Gill, S. J., Air, G., Sheriff, S., Padlan, E. A., Davies, D., Tulip, W. R., Colman, P. M., Spinelli, S., Alzari, P. M. and Poljak, R. J. (1989) Nature 342:877-883. Chothia, C., Novotny, J., Bruccoleri, R. and Karplus, M. (1985) J. Mol. Biol. 186:651 Brensing-Kuppers J, Zocher I, Thiebe R, Zachau H G. (1997). Gene. 191(2):173-81. Matsuda F, Ishii K, Bourvagnet P, Kuma K, Hayashida H, Miyata T, Honjo T. (1998) J Exp Med. 188(11):2151-62.Carter P. J. and Presta L. J. (2000) “Humanized antibodies and methods for making them” U.S. Pat. No. 6,407,213 Johnson, T. A., Rassenti, L. Z., and Kipps, T. J. (1997) J. Immunol. 158:235-246, each of which is incorporated by reference herein. Exemplary humanized variable regions embraced by the instant application are set forth in the examples.

De-immunization can also be used to decrease the immunogenicity of a binding molecule. As used herein, the term “de-immunization” includes alteration of an binding molecule to modify T cell epitopes (see, e.g., WO9852976A1, WO0034317A2). For example, VH and VL sequences from the starting antibody may analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., LT-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In one embodiment, a binding molecule of the invention is a humanized antibody or comprises a humanized antibody variable region having an acceptor human framework or substantially human acceptor framework. An “acceptor human framework” for the purposes herein is a framework comprising the amino acid sequence of a VL or VH framework derived from a human immunoglobulin framework, or from a human consensus framework. An acceptor human framework “derived from” a human immunoglobulin framework or human consensus framework may comprise the same amino acid sequence thereof, or may contain certain amino acid sequence changes. In one embodiment, the VL acceptor human framework is identical in sequence to the VL human immunoglobulin framework sequence or human consensus framework sequence.

A “human consensus framework” is a framework that represents the most commonly occurring amino acid residue in a selection of human immunoglobulin VL or VH framework sequences. Generally, the selection of human immunoglobulin VL or VH sequences is from a subgroup of variable domain sequences. Human germline sequences or germline sequences with some consensus sequence (e.g., FR4) may be considered as well.

In one embodiment, acceptor framework sequences for the light and heavy chains are identified having high similarity to the murine starting antibody sequences in canonical, interface and veneer zone residues. CDR sequences are excluded when determining similarity to germline sequences. In one embodiment, acceptor sequences have the same length CDRs if (except CDR-H3); and require a minimum number of backmutations.

In one embodiment, acceptor frameworks that are more distant from stable consensus classes are chosen in order to improve the physico-chemical properties of humanized designs.

In one embodiment, for the 105 antibody, human germline sequence huL6 (with consensus human KV3 FR4) and human gil3004688 may be used as the acceptor frameworks for light and heavy chains respectively.

In one embodiment, a humanized 105 light chain is made comprising a backmutation at amino acid position 1 (E→D; i.e., E to D). In one embodiment, a backmutation at amino acid position 21 (L→I) is made. In another embodiment, a backmutation at amino acid position 68 (G→R) is made. In yet another embodiment, a backmutation at amino acid position 86 (Y→F) is made.

In one embodiment, a first version of the humanized light chain is made comprising a backmutation at position 1. In another embodiment, a second version of the 105 light chain is made comprising a backmutation at position 1, 21, and 86. In another embodiment, a third version of the 105 light chain is made comprising a backmutation at position 1, 21, 68, and 86.

Three different versions of the humanized LT105 light chain are described below The humanized light chain of LT105 included: Germline huL6 framework//consensus human KV4 FR4//LT105 L CDRs. Backmutations described below in L1, L2, and L3 are in lowercase, bold font. CDRs, including Chothia definition, are underlined.

> LO = graft
(SEQ ID NO: 20)
EIVLTQSPATLSLSPGERATLSCRASESVDNYGISFMHWYQQKPGQAPRLLIYRASNLESGIPARFSGSGS
GTDFTLTISSLEPEDFAVYYCQQSNKDPYTFGQGTKVEIK
> L1
(SEQ ID NO: 21)
dIVLTQSPATLSLSPGERATLSCRASESVDNYGISFMHWYQQKPGQAPRLLIYRASNLESGIPARFSGSGS
GTDFTLTISSLEPEDFAVYYCQQSNKDPYTFGQGTKVEIK
> L2
(SEQ ID NO: 22)
dIVLTQSPATLSLSPGERATiSCRASESVDNYGISFMHWYQQKPGQAPRLLIYRASNLESGIPARFSGSGS
GTDFTLTISSLEPEDFAVfYCQQSNKDPYTFGQGTKVEIK
> L3
(SEQ ID NO: 23)
dIVLTQSPATLSLSPGERATiSCRASESVDNYGISFMHWYQQKPGQAPRLLIYRASNLESGIPARFSGSGS
rTDFTLTISSLEPEDFAVfYCQQSNKDPYTFGQGTKVEIK

In one embodiment, a humanized 105 heavy chain is made comprising a backmutation at amino acid position 1 (E→D). In one embodiment, a backmutation at amino acid position 2r (A→V) is made. In another embodiment, a backmutation at amino acid position 25 (S→T) is made. In yet another embodiment, a backmutation at amino acid position 37 (V→I) is made. In yet another embodiment, a backmutation at amino acid position 47 (W→G) is made. In yet another embodiment, a backmutation at amino acid position 48 (I→M) is made. In yet another embodiment, a backmutation at amino acid position 49 (S→G) is made. In yet another embodiment, a backmutation at amino acid position 67 (F→I) is made. In yet another embodiment, a backmutation at amino acid position 78 (L→F) is made. In yet another embodiment, a backmutation at amino acid position 82 (M→L) is made.

In one embodiment, a first version of the humanized 105 heavy chain is made comprising a backmutation at position 24 and 47. In another embodiment, a second version of the 105 heavy chain is made comprising a backmutation at position 24, 37, 49, 67, and 78. In another embodiment, a third version of the 105 heavy chain is made comprising a backmutation at position 1, 24, 25, 37, 47, 49, 67, and 78. In another embodiment, a fourth version of the 105 heavy chain is made comprising a backmutation at position 1, 24, 25, 37, 47, 48, 49, 67, 78, and 82.

Four different versions of the humanized LT105 heavy chain are described below The humanized heavy chain of LT105 included: gil3004688 framework//LT105 H CDRs. Backmutations described below in H1, H2, H3, and H4 are in lowercase, bold font. CDRs, including Chothia definition, are underlined.

> HO = graft
(SEQ ID NO: 24)
EVQLVESGGGLVQPGGSLRLSCAASGYSITSGYYWNWVRQAPGKGLEWISYISYDGSNNYNPSLKNRFTIS
RDSAKNSLYLHMHSLRAEDTAVYYCARDAYSYGMDYWGQGTTVTVSS
> H1
(SEQ ID NO: 25)
EVQLVESGGGLVQPGGSLRLSCAvSGYSITSGYYWNWVRQAPGKGLEgISYISYDGSNNYNPSLKNRFTIS
RDSAKNSfYLHMHSLRAEDTAVYYCARDAYSYGMDYWGQGTTVTVSS
> H2
(SEQ ID NO: 26)
EVQLVESGGGLVQPGGSLRLSCAvSGYSITSGYYWNWiRQAPGKGLEgIgYISYDGSNNYNPSLKNRiTIS
RDSAKNSfYLHMHSLRAEDTAVYYCARDAYSYGMDYWGQGTTVTVSS
> H3
(SEQ ID NO: 27)
dVQLVESGGGLVQPGGSLRLSCAvtGYSITSGYYWNWiRQAPGKGLEgIgYISYDGSNNYNPSLKNRiTIS
RDSAKNSfYLHMHSLRAEDTAVYYCARDAYSYGMDYWGQGTTVTVSS
> H4
(SEQ ID NO: 28)
dVQLVESGGGLVQPGGSLRLSCAvtGYSITSGYYWNWiRQAPGKGLEgmgYISYDGSNNYNPSLKNRiTIS
RDSAKNSfYLHlHSLRAEDTAVYYCARDAYSYGMDYWGQGTTVTVSS

As set forth above additional alterations may be made to generate alternative versions of the 105 antibody and various light and heavy chain combinations can be made. For example, in one embodiment, a binding molecule of the invention comprises the light chain of the 105 antibody version 0 or the CDRs thereof. In another embodiment, a binding molecule of the invention comprises the heavy chain of the 105 antibody version 1 or the CDRs thereof. In another embodiment, a binding molecule of the invention comprises the light chain of the 105 antibody version 0 or the CDRs thereof in combination with the heavy chain of the 105 antibody version 1 or the CDRs thereof:

L0

(SEQ ID NO: 29)

| | |
|---|---|
| 1 | EIVLTQSPAT LSLSPGERAT LSCRASESVD NYGISFMHWY |
| | QQKPGQAPRL |
| 51 | LIYRASNLES GIPARFSGSG SGTDFTLTIS SLEPEDFAVY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

H1

(SEQ ID NO: 30)

| | |
|---|---|
| 1 | EVQLVESGGG LVQPGGSLRL SCAVSGYSIT SGYYWNWVRQ |
| | APGKGLEGIS |
| 51 | YISYDGSNNY NPSLKNRFTI SRDSAKNSFY LHMHSLRAED |
| | TAVYYCARDA |
| 101 | YSYGMDYWGQ GTTVTVSSAS TKGPSVFPLA PSSKSTSGGT |
| | AALGCLVKDY |
| 151 | FPEPVTVSWN SGALTSGVHT FPAVLQSSGL YSLSSVVTVP |
| | SSSLGTQTYI |
| 201 | CNVNHKPSNT KVDKKVEPKS CDKTHTCPPC PAPELLGGPS |
| | VFLFPPKPKD |
| 251 | TLMISRTPEV TCVVVDVSHE DPEVKFNWYV DGVEVHNAKT |
| | KPREEQYNST |
| 301 | YRVVSVLTVL HQDWLNGKEY KCKVSNKALP APIEKTISKA |
| | KGQPREPQVY |
| 351 | TLPPSRDELT KNQVSLTCLV KGFYPSDIAV EWESNGQPEN |
| | NYKTTPPVLD |
| 401 | SDGSFFLYSK LTVDKSRWQQ GNVFSCSVMH EALHNHYTQK |
| | SLSLSPG |

In another embodiment, a binding molecule of the invention comprises the light chain of version A of the 105 antibody or the CDRs thereof. In another embodiment, a binding molecule of the invention comprises the light chain of version B of the 105 antibody or the CDRs thereof. In another embodiment, a binding molecule of the invention comprises the light chain of version C of the 105 antibody or the CDRs thereof. For example, in one embodiment, such a light chain can be paired with a heavy chain version of a 105 antibody.

(SEQ ID NO: 31)

| | |
|---|---|
| 1 | EIVLTQSPAT LSLSPGERAT LSCRASESVD NYGISFMHWY |
| | QQKPGQAPRL |
| 51 | LIYKASNLES GIPARFSGSG SGTDFTLTIS SLEPEDFAVY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

Version B

(SEQ ID NO: 32)

| | |
|---|---|
| 1 | EIVLTQSPAT LSLSPGERAT LSCRASESVD NYGISFMHWY |
| | QQKPGQAPRL |
| 51 | LIYRASSLES GIPARFSGSG SGTDFTLTIS SLEPEDFAVY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

Version C

(SEQ ID NO: 33)

| | |
|---|---|
| 1 | EIVLTQSPAT LSLSPGERAT LSCRASESVD NYGISFMHWY |
| | QQKPGQAPRL |
| 51 | LIYKASSLES GIPARFSGSG SGTDFTLTIS SLEPEDFAVY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

In another embodiment, a binding molecule of the invention comprises the light chain of the 105 antibody version 10 or the CDRs thereof. In another embodiment, a binding molecule of the invention comprises the heavy chain of the 105 antibody version 1 or the CDRs thereof. In another embodiment, a binding molecule of the invention comprises the light chain of the 105 antibody version 10 or the CDRs thereof in combination with the heavy chain of the 105 antibody version 1 or the CDRs thereof:

L10

(SEQ ID NO: 34)

| | |
|---|---|
| 1 | AIQLTQSPSS LSASVGDRVT ITCRASESVD NYGISFMHWY |
| | QQKPGKAPKL |
| 51 | LIYKASSLES GVPSRFSGSG SGTDFTLTIS SLQPEDFATY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

In another embodiment, a binding molecule of the invention comprises the light chain of the 105 antibody version 12 or 13 or the CDRs thereof. In another embodiment, a binding molecule of the invention comprises the heavy chain of the 105 antibody version 1 or the CDRs thereof. In another embodiment, a binding molecule of the invention comprises the light chain of the 105 antibody version 12 or 13 or the CDRs thereof in combination with the heavy chain of the 105 antibody version 1 or the CDRs thereof:

L12

(SEQ ID NO: 35)

| | |
|---|---|
| 1 | DIQLTQSPSS LSASVGDRVT ITCRASESVD NYGISFMHWY |
| | RQKPGKAPKL |
| 51 | LIYKASSLES GVPSRFSGRG SGTDFTLTIS SLQPEDFATY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

L13

(SEQ ID NO: 36)

| | |
|---|---|
| 1 | DIRLTQSPSS LSASVGQRVT ISCRASESVD NYGISFMHWY |
| | RQKPGKAPKL |
| 51 | LIYKASSLES GVPSRFSGRG SGTDFTLTIS SLQPEDFATY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

In another embodiment, a binding molecule of the invention comprises the heavy chain of version 11 or 14 of the 105 antibody or the CDRs thereof, e.g., in combination with a light chain version of the 105 antibody.

H11

(SEQ ID NO: 37)

| | |
|---|---|
| 1 | EVQLVESGGG LVQPRGSLRL SCAVSGYSIT SGYYWNWIRQ |
| | APGKGLEWVS |
| 51 | YISYDGSNNY NPSLKNRFTI SRDNSKNTFY LQMNNLRAED |
| | TAAYYCARDA |
| 101 | YSYGMDYWGQ GTTVTVSSAS TKGPSVFPLA PSSKSTSGGT |
| | AALGCLVKDY |
| 151 | FPEPVTVSWN SGALTSGVHT FPAVLQSSGL YSLSSVVTVP |
| | SSSLGTQTYI |
| 201 | CNVNHKPSNT KVDKKVEPKS CDKTHTCPPC PAPELLGGPS |
| | VFLFPPKPKD |
| 251 | TLMISRTPEV TCVVVDVSHE DPEVKFNWYV DGVEVHNAKT |
| | KPREEQYNST |
| 301 | YRVVSVLTVL HQDWLNGKEY KCKVSNKALP APIEKTISKA |
| | KGQPREPQVY |
| 351 | TLPPSRDELT KNQVSLTCLV KGFYPSDIAV EWESNGQPEN |
| | NYKTTPPVLD |
| 401 | SDGSFFLYSK LTVDKSRWQQ GNVFSCSVMH EALHNHYTQK |
| | SLSLSPG |

H14

(SEQ ID NO: 38)

| | |
|---|---|
| 1 | EVQLQESGGG LVKPRGSLRL SCAVSGYSIT SGYYWNWIRQ |
| | APGKGLEWVS |
| 51 | YISYDGSNNY NPSLKNRFSI SRDNSKNTFY LKMNRLRAED |
| | SAAYYCARDA |
| 101 | YSYGMDYWGQ GTTVTVSSAS TKGPSVFPLA PSSKSTSGGT |
| | AALGCLVKDY |
| 151 | FPEPVTVSWN SGALTSGVHT FPAVLQSSGL YSLSSVVTVP |
| | SSSLGTQTYI |
| 201 | CNVNHKPSNT KVDKKVEPKS CDKTHTCPPC PAPELLGGPS |
| | VFLFPPKPKD |
| 251 | TLMISRTPEV TCVVVDVSHE DPEVKFNWYV DGVEVHNAKT |
| | KPREEQYNST |
| 301 | YRVVSVLTVL HQDWLNGKEY KCKVSNKALP APIEKTISKA |
| | KGQPREPQVY |
| 351 | TLPPSRDELT KNQVSLTCLV KGFYPSDIAV EWESNGQPEN |
| | NYKTTPPVLD |
| 401 | SDGSFFLYSK LTVDKSRWQQ GNVFSCSVMH EALHNHYTQK |
| | SLSLSPG |

In one embodiment, for the 102 antibody, human germline sequence huA3 (with consensus HUMKV2 FR4) and human germline sequence huVH3-11 (with consensus HUMHV3 FR4) are used.

One version of the variable light reshaped chain was designed, and four versions of the variable heavy reshaped chain was designed, in addition to the light and heavy CDR graft sequences. For the heavy chain, the first version contains the fewest backmutations and the next versions contain more backmutations (i.e. they are the least “humanized”). The murine A113 was substituted by 5113 (present in human HV FR4) in all versions of the heavy chain, and was not analyzed as a backmutation. Numbering is according to the Kabat scheme.

In one embodiment, a reshaped light chain of humanized LT102 (huLT102) includes a germline huA3 framework, consensus human KV2 FR4, and LT102 L CDRs. The backmutation in the light chain of hu102 included: I2V. V2 is a canonical residue supporting CDR-L1.

Exemplary humanized LT102 light chain sequence is described below (for details regarding backmutation see above). The humanized light chain of LT102 included: Germline huA3 framework//consensus human KV2 FR4//LT102 L CDRs. Backmutations are in lowercase bold font. CDRs, including Chothia definition, are underlined.

> L0 = graft

(SEQ ID NO: 39)

DIVMTQSPLSLPVTPGEPASISCRSSQNIVHSNGNTYLEWYLQKPGQSPQ
LLIYKVSNRFSGVPDRFSGSGSGTDFTLKISRVEAEDVGVYYCFQGSHFP
WTFGQGTKVEIK
> L1

(SEQ ID NO: 40)

DvVMTQSPLSLPVTPGEPASISCRSSQNIVHSNGNTYLEWYLQKPGQSPQ
LLIYKVSNRFSGVPDRFSGSGSGTDFTLKISRVEAEDVGVYYCFQGSHFP
WTFGQGTKVEIK

The four different versions of the humanized LT102 heavy chain are described below The humanized heavy chain of LT102 included: Germline huVH3-11 framework//consensus human HV3 FR4//LT102 H CDRs. Backmutations described below in H1, H2, H3, and H4 are in lowercase, bold font. CDRs, including Chothia definition, are underlined.

> H0 = graft

(SEQ ID NO: 41)

QVQLVESGGGLVKPGGSLRLSCAASGFTFSDYYMYWIRQAPGKGLEWVST
IGDGTSYTHYPDSVQGRFTISRDNAKNSLYLQMNSLRAEDTAVYYCARDL
GTGPFAYWGQGTLVTVSS
> H1

(SEQ ID NO: 42)

QVQLVESGGGLVKPGGSLRLSCAvSGFTFSDYYMYWIRQAPGKGLEWVST
IGDGTSYTHYPDSVQGRFTISRDNAKNSLYLQMNSLRAEDTAVYYCARDL
GTGPFAYWGQGTLVTVSS
> H2

(SEQ ID NO: 43)

eVQLVESGGGLVKPGGSLRLSCAvSGFTFSDYYMYWIRQAPGKGLEWVST
IGDGTSYTHYPDSVQGRFTISRDyAKNSLYLQMNSLRAEDTAVYYCARDL
GTGPFAYWGQGTLVTVSS
> H3

(SEQ ID NO: 44)

eVkLVESGGGLVKPGGSLRLSCAvSGFTFSDYYMYWIRQAPGKGLEWVST
IGDGTSYTHYPDSVQGRFTISRDyAKNSLYLQMNSLRAEDTAVYYCARDL
GTGPFAYWGQGTLVTVSS
> H4

(SEQ ID NO: 45)

eVkLVESGGGLVKPGGSLRLSCAvSGFTFSDYYMYWIRQAPGKGLEWVST
IGDGTSYTHYPDSVQGRFTISRDyAtNnLYLQMNSLRAEDTAVYYCARDL
GTGPFAYWGQGTLVTVSS

In one embodiment, a humanized 102 light chain is made comprising a backmutation at amino acid position 2 (I→V).

In one embodiment, a humanized 102 heavy chain is made comprising a backmutation at amino acid position 24 (A→V). In one embodiment, a humanized 102 heavy chain is made comprising a backmutation at amino acid position 73 (N→Y). In one embodiment, a humanized 102 heavy chain is made comprising a backmutation at amino acid position 3 (Q→K). In one embodiment, a humanized 102 heavy chain is made comprising a backmutation at amino acid position K→T). In one embodiment, a humanized 102 heavy chain is made comprising a backmutation at amino acid position 77 S→N).

In one embodiment, a first version of the humanized 102 heavy chain is made comprising a backmutation at position 24. In another embodiment, a second version of the 102 heavy chain is made comprising a backmutation at position 24, 1, and 73. In another embodiment, a third version of the 102 heavy chain is made comprising a backmutation at position 24, 1, 73, and 3. In another embodiment, a fourth version of the 102 heavy chain is made comprising a backmutation at position 24, 1, 73, 3, 75, and 77.

C. Effector Functions and Fc Modifications

LT binding molecules of the invention may comprise a constant region which mediates one or more effector functions. For example, binding of the C1 component of complement to an antibody constant region may activate the complement system thereby causing complement dependent cytotoxicity of target cells. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with an Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Certain embodiments of the invention include LT binding molecules in which at least one amino acid in one or more of the constant region domains has been deleted or otherwise altered so as to provide desired biochemical characteristics such as: reduced effector function(s), increased effector function(s), improved ability to non-covalently dimerize, increased ability to localize at the site of a tumor, reduced serum half-life, or increased serum half-life when compared with a whole, unaltered antibody of approximately the same immunogenicity. For example, certain binding molecules for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain LT binding molecules, an anti-LT binding site may be fused to an Fc portion. In one embodiment, the Fc portion may be a wild-type Fc portion derived from an antibody molecule. In another embodiment, the Fc portion may be mutated to change (e.g., increase or decrease) effector function using techniques known in the art. For example, the deletion or inactivation (through point mutations or other means) of a constant region domain may reduce Fc receptor binding of the circulating modified binding molecule thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or flexibility. The resulting physiological profile, bioavailability and other biochemical effects of the modifications, such as tumor localization, biodistribution and serum half-life, may easily be measured and quantified using well known immunological techniques without undue experimentation.

In certain embodiments, an Fc domain employed in a binding polypeptide of the invention is an Fc variant. As used herein, the term “Fc variant” refers to an Fc domain having at least one amino acid substitution relative to the wild-type Fc domain from which said Fc domain is derived. For example, wherein the Fc domain is derived from a human IgG1 antibody, the Fc variant of said human IgG1 Fc domain comprises at least one amino acid substitution relative to the wild-type Fc domain, e.g., designed to alter effector function or half-life of the binding molecule.

The amino acid substitution(s) of an Fc variant may be located at any position (ie., any EU convention amino acid position) within the Fc domain. In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

The binding polypeptides of the invention may employ any art-recognized Fc variant which is known to impart an improvement (e.g., reduction or enhancement) in effector function and/or FcR binding. Said Fc variants may include, for example, any one of the amino acid substitutions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO05/070963A1, WO05/077981A2, WO05/092925A2, WO05/123780A2, WO06/019447A1, WO06/047350A2, and WO06/085967A2 or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784, each of which is incorporated by reference herein.

The certain embodiments, a binding polypeptide of the invention comprising an Fc variant polypeptide comprising an amino acid substitution which alters the antigen-independent effector functions of the antibody, in particular the circulating half-life of the antibody. Such binding polypeptides exhibit either increased or decreased binding to FcRn when compared to binding polypeptides lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered polypeptide is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization the brain, kidney, and/or liver is desired. In one exemplary embodiment, the altered polypeptides of the invention exhibit reduced transport across the epithelium of kidney glomeruli from the vasculature. In another embodiment, the altered polypeptides of the invention exhibit reduced transport across the blood brain barrier (BBB) from the brain, into the vascular space. In one embodiment, a binding polypeptide with altered FcRn binding comprises an Fc domain having one or more amino acid substitutions within the “FcRn binding loop” of an Fc domain. The FcRn binding loop is comprised of amino acid residues 280-299 (according to EU numbering). In other embodiment, a binding polypeptide of the invention having altered FcRn binding affinity comprises an Fc domain having one or more amino acid substitutions within the 15 \acute{Å} FcRn “contact zone.” As used herein, the term 15 \acute{Å} FcRn “contact zone” includes residues at the following positions 243-261, 275-280, 282-293, 302-319, 336-348, 367, 369, 372-389, 391, 393, 408, 424, 425-440 (EU numbering). In preferred embodiments, a binding polypeptide of the invention having altered FcRn binding affinity comprises an Fc domain having one or more amino acid substitutions at any one of the following positions: 256, 277-281, 283-288, 303-309, 313, 338, 342, 376, 381, 384, 385, 387, 434, and 438. Exemplary amino acid substitutions which altered FcRn binding activity are disclosed in International PCT Publication No. WO05/047327 which is incorporated by reference herein.

In other embodiments, certain binding molecules for use in the diagnostic and treatment methods described herein have a constant region, e.g., an IgG4 heavy chain constant region, which is altered to reduce or eliminate glycosylation. For example, a binding polypeptide of the invention may also comprise an Fc variant comprising an amino acid substitution which alters the glycosylation of the binding polypeptide. For example, said Fc variant may have reduced glycosylation (e.g., N- or O-linked glycosylation) or may comprise an altered glycoform of the wild-type Fc domain (e.g., a low fucose or fucose-free glycan). Such low fucose or afucosylated forms of molecules may be made using alternative cell lines known in the art to produce such altered forms. In one embodiment, the Fc variant is afucosylated.

In exemplary embodiments, the Fc variant comprises reduced glycosylation of the N-linked glycan normally found at amino acid position 297 (EU numbering). In another embodiment, the binding polypeptide has an amino acid substitution near or within a glycosylation motif, for example, an N-linked glycosylation motif that contains the amino acid sequence NXT or NXS. In a particular embodiment, the binding polypeptide comprises an Fc variant with an amino acid substitution at amino acid position 228 or 299 (EU numbering). In more particular embodiments, the binding molecule comprises an IgG4 constant region comprising an S228P and a T299A mutation (EU numbering).

Exemplary amino acid substitutions which confer reduce or altered glycosylation are disclosed in International PCT Publication No. WO05/018572, which is incorporated by reference herein. In preferred embodiments, the binding molecules of the invention are modified to eliminate glycosylation. Such binding molecules may be referred to as “agly” binding molecules (e.g. “agly” antibodies). While not being bound by theory, it is believed that “agly” binding molecules may have an improved safety and stability profile in vivo. Exemplary agly binding molecules comprise an aglycosylated Fc region of an IgG4 antibody (“IgG4.P”) which is devoid of Fc-effector function thereby eliminating the potential for Fc mediated toxicity to the normal vital organs that express LT. In particular embodiments, agly binding molecules of the invention may comprise the IgG4.P or IgG4PE constant region as known in the art.

### V. Methods of Making Binding Molecules

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode separate chains of a binding molecule of the invention, e.g., the light and the heavy chains of an antibody, may be made, either simultaneously or separately, using reverse transcriptase and DNA polymerase in accordance with well known methods. For example, PCR may be initiated by consensus constant region primers or by more specific primers based on the published DNA and amino acid sequences. As discussed above, PCR also may be used to isolate DNA clones encoding separate binding molecule chains. In this case the libraries may be screened by consensus primers or larger homologous probes, such as mouse constant region probes. DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis. Following manipulation of the isolated genetic material to provide binding molecules of the invention, the polynucleotides encoding the LT binding molecules are typically inserted in an expression vector for introduction into host cells that may be used to produce the desired quantity of LT binding molecule.

Recombinant expression of a binding molecule, e.g., a heavy or light chain of an antibody which binds to a target molecule described herein, e.g., LT, requires construction of an expression vector containing a polynucleotide that encodes the binding molecule. Once a polynucleotide encoding a binding molecule (or a chain or portion thereof) of the invention has been obtained, the vector for the production of the binding molecule may be produced by recombinant DNA technology using techniques well known in the art. Thus, methods for preparing a protein by expressing a polynucleotide containing a binding molecule encoding nucleotide sequence are described herein. Methods which are well known to those skilled in the art can be used to construct expression vectors containing binding molecule coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a binding molecule of the invention, or a chain or domain thereof, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the nucleotide encoding the binding molecule (or chain or domain thereof) may be cloned into such a vector for expression of the entire binding molecule.

Where the binding molecule of the invention is a dimer, the host cell may be co-transfected with two expression vectors of the invention, the first vector encoding a first polypeptide monomer and the second vector encoding a second polypeptide monomer. The two vectors may contain identical selectable markers which enable equal expression of the monomers. Alternatively, a single vector may be used which encodes both monomers. In embodiments the monomers are antibody light and heavy chains, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the monomers of a binding molecule may comprise cDNA or genomic DNA. The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals. In particularly preferred embodiments the cloned variable region genes are inserted into an expression vector along with the heavy and light chain constant region genes (preferably human) synthetic as discussed above. In one embodiment, this is effected using a proprietary expression vector of Biogen IDEC, Inc., referred to as NEOSPLA (disclosed in U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the binding molecules of the invention may be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of LT binding molecules thereof in eukaryotic host cells. Compatible IRES sequences are disclosed in U.S. Pat. No. 6,193,980 which is also incorporated herein. Those skilled in the art will appreciate that such expression systems may be used to effectively produce the full range of LT binding molecules disclosed in the instant application.

More generally, once the vector or DNA sequence encoding a monomeric subunit of the LT binding molecule has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the binding molecule, and assayed for binding molecule synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques and the transfected cells are then cultured by conventional techniques to produce a binding molecule for use in the methods described herein. Thus, the invention includes host cells containing a polynucleotide encoding a binding molecule of the invention, or a monomer or chain thereof, operably linked to a heterologous promoter. In preferred embodiments for the expression of double-chained or dimeric binding molecules, vectors which separately encode binding molecule chains may be co-expressed in the host cell for expression of the entire binding molecule, as detailed below.

As used herein, “host cells” refers to cells which harbor vectors constructed using recombinant DNA techniques and encoding at least one heterologous gene. In descriptions of processes for isolation of binding molecules from recombinant hosts, the terms “cell” and “cell culture” are used interchangeably to denote the source of binding molecule unless it is clearly specified otherwise. In other words, recovery of polypeptide from the “cells” may mean either from spun down whole cells, or from the cell culture containing both the medium and the suspended cells.

A variety of host-expression vector systems may be utilized to express binding molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing binding molecule coding sequences; yeast (e.g., Saccharomyces, Pichia) transformed with recombinant yeast expression vectors containing binding molecule coding sequences; insect cell systems infected with recombinant virus expression vectors (e.g., baculovirus) containing binding molecule coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing binding molecule coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant binding molecules, are used for the expression of a recombinant binding molecule. For example, mammalian cells such as Chinese hamster ovary cells (CHO) in conjunction with a vector such as the major intermediate early gene promoter element from human cytomegalovirus is an effective expression system for antibodies and other binding molecules (Foecking et al., Gene 45:101 (1986); Cockett et al., Bio/Technology 8:2 (1990)).

The host cell line used for protein expression is often of mammalian origin; those skilled in the art are credited with ability to preferentially determine particular host cell lines which are best suited for the desired gene product to be expressed therein. Exemplary host cell lines include, but are not limited to, CHO (Chinese Hamster Ovary), DG44 and DUXB11 (Chinese Hamster Ovary lines, DHFR minus), HELA (human cervical carcinoma), CVI (monkey kidney line), COS (a derivative of CVI with SV40 T antigen), VERY, BHK (baby hamster kidney), MDCK, 293, WI38, R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express the binding molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the binding molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, anti-metabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of a binding molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing the binding molecule is amplifiable, increase in the level of inhibitor present in culture of host cell will increase the number of copies of the marker gene. Since the amplified region is associated with the binding molecule, production of the binding molecule will also increase (Crouse et al., Mol. Cell. Biol. 3:257 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding LT binding molecules of the invention can also be expressed non-mammalian cells such as bacteria or insect or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of binding molecules are desired, the subunits will then self-assemble into tetravalent binding molecules (e.g. tetravalent antibodies (WO02/096948A2)).

In bacterial systems, a number of expression vectors may be advantageously selected depending upon the use intended for the binding molecule being expressed. For example, when a large quantity of such a protein is to be produced, for the generation of pharmaceutical compositions of a binding molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the binding molecule coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85:12 (1977)). The presence of the trp1 lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

Once a binding molecule of the invention has been recombinantly expressed, it may be purified by any method known in the art for purification of a binding molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of binding molecules (e.g. antibodies) of the invention is disclosed in US 2002 0123057 A1.

### VI. Methods of Treatment Using Compositions Comprising Binding Molecules which Bind to LT

One embodiment of the present invention provides methods for treating a subject that would benefit from administration of an anti-LT binding molecule the method comprising, consisting essentially of, or consisting of administering to the animal an effective amount of a binding molecule or composition of the invention described herein.

In one embodiment, a binding molecule of the invention is administered to a subject suffering from a disorder associated with inflammation or an autoimmune response. In one embodiment, to binding molecule of the invention is administered to a subject suffering from cancer.

Exemplary inflammatory or autoimmune disorders include organ-specific diseases (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease that can affect multiple organ systems (for example, systemic lupus erythematosus (SLE), rheumatoid arthritis, polymyositis, etc.). In one embodiment, an autoimmune or inflammatory disorder for treatment with a binding molecule of the invention is one that has an ectopic lymphoid manifestation.

Exemplary autoimmune or inflammatory diseases include, for example, rheumatoid arthritis, Sjogren's syndrome, scleroderma, lupus such as SLE and lupus nephritis, polymyositis/dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and ANCA-associated vasculitis, including Churg-Strauss vasculitis, Wegener's granulomatosis, and microscopic polyangiitis), autoimmune neurological disorders (such as, for example, multiple sclerosis (MS), RRMS, SPMS, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, dermatomtositis, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, RA, IBD, including Crohn's disease and ulcerative colitis, ANCA-associated vasculitis, lupus, MS, Sjogren's syndrome, Graves' disease, IDDM, pernicious anemia, thyroiditis, and glomerulonephritis. Still more preferred are RA, IBD, lupus, and MS, and more preferred RA and IBD, and most preferred RA.

Exemplary non-autoimmune indications include follicular lymphoma, atherosclerosis, viral-induced hepatitis, bronchial asthma, and viral shock syndrome.

In one embodiment, the subject binding molecules are used to treat rheumatoid arthritis. As used herein, “rheumatoid arthritis” or “RA” refers to a recognized disease state that may be diagnosed according to the 2000 revised American Rheumatoid Association criteria for the classification of RA, or any similar criteria, and includes active, early, and incipient RA, as defined below. Physiological indicators of RA include symmetric joint swelling, which is characteristic though not invariable in rheumatoid arthritis. Fusiform swelling of the proximal interphalangeal (PIP) joints of the hands as well as metacarpophalangeal (MCP), wrists, elbows, knees, ankles, and metatarsophalangeal (MTP) joints are commonly affected and swelling is easily detected. Pain on passive motion is the most sensitive test for joint inflammation, and inflammation and structural deformity often limit the range of motion for the affected joint. Typical visible changes include ulnar deviation of the fingers at the MCP joints, hyperextension, or hyperflexion of the MCP and PIP joints, flexion contractures of the elbows, and subluxation of the carpal bones and toes. The subject with RA may be resistant to DMARDs, in that the DMARDs are not effective or fully effective in treating symptoms.

In one embodiment, candidates for therapy according to this invention include those who have experienced an inadequate response to previous or current treatment with TNF inhibitors.

In one embodiment, a binding molecule of the invention is used to treat active rheumatoid arthritis. A patient with “active rheumatoid arthritis” means a patient with active and not latent symptoms of RA. Subjects with “early active rheumatoid arthritis” are those subjects with active RA diagnosed for at least eight weeks but no longer than four years, according to the revised 1987 ACR criteria for the classification of RA. Subjects with “early rheumatoid arthritis” are those subjects with RA diagnosed for at least eight weeks but no longer than four years, according to the revised 1987 ACR criteria for classification of RA. Early RA includes, for example, juvenile-onset RA, juvenile idiopathic arthritis (JIA), or juvenile RA (JRA).

In one embodiment, a binding molecule of the invention is used to treat incipient rheumatoid arthritis. Patients with “incipient RA” have early polyarthritis that does not fully meet ACR criteria for a diagnosis of RA, but is associated with the presence of RA-specific prognostic biomarkers such as anti-CCP and shared epitope. They include patients with positive anti-CCP antibodies who present with polyarthritis, but do not yet have a diagnosis of RA, and are at high risk for going on to develop bonafide ACR criteria RA (95% probability).

“Joint damage” is used in the broadest sense and refers to damage or partial or complete destruction to any part of one or more joints, including the connective tissue and cartilage, where damage includes structural and/or functional damage of any cause, and may or may not cause joint pain/arthalgia. It includes, without limitation, joint damage associated with or resulting from inflammatory joint disease as well as non-inflammatory joint disease. This damage may be caused by any condition, such as an autoimmune disease, especially arthritis, and most especially RA. Exemplary such conditions include acute and chronic arthritis, RA including juvenile-onset RA, juvenile idiopathic arthritis (JIA), or juvenile RA (JRA), and stages such as rheumatoid synovitis, gout or gouty arthritis, acute immunological arthritis, chronic inflammatory arthritis, degenerative arthritis, type II collagen-induced arthritis, infectious arthritis, septic arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), rheumatic autoimmune disease other than RA, and significant systemic involvement secondary to RA (including but not limited to vasculitis, pulmonary fibrosis or Felty's syndrome). For purposes herein, joints are points of contact between elements of a skeleton (of a vertebrate such as an animal) with the parts that surround and support it and include, but are not limited to, for example, hips, joints between the vertebrae of the spine, joints between the spine and pelvis (sacroiliac joints), joints where the tendons and ligaments attach to bones, joints between the ribs and spine, shoulders, knees, feet, elbows, hands, fingers, ankles, and toes, but especially joints in the hands and feet.

In one embodiment, the subject has never been previously treated with drug(s), such as immunosuppressive agent(s), to treat the disorder, and in a particular embodiment has never been previously treated with a TNF antagonist. In an alternative embodiment, the subject has been previously treated with drug(s) to treat the disorder, including with a TNF antagonist.

In a still further aspect, the patient has relapsed with the disorder. In an alternative embodiment, the patient has not relapsed with the disorder.

In another aspect, the antibody herein is the only medicament administered to the subject to treat the disorder. In an alternative aspect, the binding molecule herein is one of the medicaments used to treat the disorder.

In a further aspect, the subject only has RA as an autoimmune disorder.

Alternatively, the subject only has MS as an autoimmune disorder. Still alternatively, the subject only has lupus, or ANCA-associated vasculitis, or Sjogren's syndrome as an autoimmune disorder.

### VIII. Pharmaceutical Compositions and Administration Methods

Methods of preparing and administering LT-specific binding molecules to a subject in need thereof are well known to or are readily determined by those skilled in the art. The route of administration of the binding molecule may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, binding molecules can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding molecule of the invention) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 (US-2002-0102208 A1), which is incorporated herein by reference in its entirety. Such articles of manufacture will preferably have labels or package inserts indicating that the associated compositions are useful for treating a subject suffering from, or predisposed to autoimmune or neoplastic disorders.

Effective doses of the compositions of the present invention, for treatment of hyperproliferative disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of hyperproliferative disorders with an antibody or fragment thereof, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

LT-specific binding molecule disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, binding molecules can be administered as a sustained release formulation, in which case less frequent administration is required. Dosage and frequency vary depending on the half-life of the antibody in the patient. The half-life of a binding molecule can also be prolonged via fusion to a stable polypeptide or moiety, e.g., albumin or PEG. In general, humanized antibodies show the longest half-life, followed by chimeric antibodies and nonhuman antibodies. In one embodiment, the binding molecules of the invention can be administered in unconjugated form, In another embodiment, the binding molecules for use in the methods disclosed herein can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding an LT-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. In some methods, agents are injected directly into a particular tissue where LTbR-expressing cells have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

LT binding molecules can optionally be administered in combination with other agents that are effective in treating the disorder or condition in need of treatment (e.g., prophylactic or therapeutic).

In keeping with the scope of the present disclosure, LT-specific binding molecules of the present invention may be administered to a human or other animal in accordance with the aforementioned methods of treatment in an amount sufficient to produce a therapeutic or prophylactic effect. The LT-specific antibodies binding molecules of the present invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding molecules according to the present invention may prove to be particularly effective.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All of the references cited above, as well as all references cited herein, are incorporated herein by reference in their entireties.

### EXAMPLES

### Example 1

### Cloning of Anti-Lymphotoxin Antibodies

Mouse monoclonal antibodies (mAbs) directed against a human lymphotoxin (LT) were prepared by injecting mice with LTα1β2 present on beads. LTα1β2 was linked to beads using art recognized techniques (using anti-myc antibody or via CnBr fixation to the bead surface).

Total cellular RNA from murine hybridoma cells was prepared using a Qiagen RNeasy mini kit following the manufacturer's recommended protocol. cDNAs encoding the variable regions of the heavy and light chains were cloned by RT-PCR from total cellular RNA, using random hexamers for priming of first strand cDNA. For PCR amplification of the murine immunoglobulin variable domains with intact signal sequences, a cocktail of degenerate forward primers hybridizing to multiple murine immunoglobulin gene family signal sequences and a single back primer specific for the 5′ end of the murine constant domain. PCR used Clontech Advantage 2 Polymerase mix following the manufacturer's recommended protocol. The PCR products were gel-purified and subcloned into Invitrogen's pCR2.1TOPO vector using their TOPO cloning kit following the manufacturer's recommended protocol. Inserts from multiple independent subclones were sequenced to establish a consensus sequence. Deduced mature immunoglobulin N-termini were consistent with those determined by Edman degradation from the hybridoma.

Assignment to specific subgroups was based upon BLAST analysis using consensus immunoglobulin variable domain sequences from the Kabat database (Kabat et al. (1991) Sequences of Proteins of Immunological Interest. 5th Edition, U.S. Dept. of Health and Human Services. U.S. Govt. Printing Office.). CDRs below are designated using the Kabat definitions.

### mAb A0D9

Shown below is the A0D9 mature heavy chain variable domain protein sequence, with CDRs underlined:

(SEQ ID NO: 46)

| | |
|---|---|
| 1 | QVQLKQSGPG LVQPSQSLSI TCTVSGFSLS TYGVHWVRQF |
| | PGKGLEWLGV |
| 51 | IWRGGNTNYN AAFMSRLTIS KDNSKSQVFF KMNSLQAKDT |
| | AIYYCVRNQI |
| 101 | YDGYYDYAMD YWGQGTSVTV SS |

The A0D9 heavy chain is a murine subgroup I(B) heavy chain.

Shown below is the DNA sequence of the A0D9 heavy chain variable domain (from pYL460), with its signal sequence underlined (heavy chain encoded signal is MAVLGLLFCLVTFPSCVLS (SEQ ID NO:47)):

(SEQ ID NO: 48)

| | |
|---|---|
| 1 | ATGGCTGTCC TGGGGCTGCT CTTCTGCCTG GTGACATTCC |
| | CAAGCTGTGT |
| 51 | CCTGTCCCAG GTGCAGCTGA AGCAGTCAGG ACCTGGCCTA |
| | GTGCAGCCCT |
| 101 | CACAGAGCCT GTCCATCACC TGCACAGTCT CTGGTTTCTC |
| | ATTATCTACC |
| 151 | TATGGTGTCC ACTGGGTTCG CCAGTTTCCA GGAAAGGGTC |
| | TGGAGTGGCT |
| 201 | GGGAGTGATA TGGAGAGGTG GAAACACAAA CTATAATGCA |
| | GCTTTCATGT |
| 251 | CCAGACTGAC CATCAGCAAG GACAATTCCA AGAGTCAAGT |
| | TTTCTTTAAA |
| 301 | ATGAACAGTC TGCAAGCTAA AGACACAGCC ATATATTATT |
| | GTGTCAGAAA |
| 351 | CCAGATCTAT GATGGTTACT ACGACTATGC TATGGACTAC |
| | TGGGGTCAGG |
| 401 | GAACCTCAGT CACCGTCTCC TCA |

Shown below is the A0D9 mature light chain variable domain protein sequence, with CDRs underlined:

(SEQ ID NO: 49)

| | |
|---|---|
| 1 | DIKMTQSPSS MYASLGERVT ITCKASQDIN TYLNWLQQKP |
| | GKSPKTLIYR |
| 51 | ANRLVDGVPS RFSGRGSGQD YSLTISSLEY EDVGIYYCLH |
| | YDAFPWTFGG |
| 101 | GTKLEIK |

The A0D9 light chain is a murine subgroup V kappa light chain.

Shown below is the DNA sequence of the mature light chain variable domain (from pYL463), with its signal sequence underlined (light chain encoded signal is MRAPAQFFGFLLLWFPGIKC (SEQ ID NO: 50)):

(SEQ ID NO: 51)

| | |
|---|---|
| 1 | ATGAGGGCCC CTGCTCAGTT TTTTGGCTTC TTGTTGCTCT |
| | GGTTTCCAGG |
| 51 | TATCAAATGT GACATCAAGA TGACCCAGTC TCCATCTTCC |
| | ATGTATGCAT |
| 101 | CTCTAGGAGA GAGAGTCACT ATCACTTGCA AGGCGAGTCA |
| | GGACATTAAT |
| 151 | ACCTATTTAA ACTGGCTCCA GCAGAAACCA GGGAAATCTC |
| | CTAAGACCCT |
| 201 | GATCTATCGT GCAAACAGAT TGGTAGATGG GGTCCCATCA |
| | AGGTTCAGTG |
| 251 | GCCGTGGATC TGGGCAAGAT TATTCTCTCA CCATCAGCAG |
| | CCTGGAATAT |
| 301 | GAAGATGTGG GAATTTATTA TTGTCTACAC TATGATGCAT |
| | TTCCGTGGAC |
| 351 | GTTCGGCGGA GGCACCAAGC TGGAAATCAA A |

### mAb A1D5

Shown below is the A1D5 mature heavy chain variable domain protein sequence, with CDRs underlined:

(SEQ ID NO: 52)

| | |
|---|---|
| 1 | EVQLQQSGPE LVKPGASVKI SCKASGYSFT GYFMNWMRQS |
| | HGKSLEWIGR |
| 51 | INPYNGDSFY NQKFKDKATL TVDKSSTTAH MELLSLTSED |
| | SAVYYCGRGY |
| 101 | DAMDYWGQGT SVTVSS |

### The A1D5 Heavy Chain is a Murine Subgroup I(B) Heavy Chain.

Shown below is the DNA sequence of the A1D5 heavy chain variable domain (from pYL338), with its signal sequence underlined (heavy chain encoded signal is MGWSCVMLFLL SVTVGVFS (SEQ ID NO:53)):

(SEQ ID NO: 54)

| | |
|---|---|
| 1 | ATGGGATGGA GCTGTGTAAT GCTCTTTCTC CTGTCAGTAA |
| | CTGTAGGTGT |
| 51 | GTTTTCTGAG GTTCAGCTGC AGCAGTCTGG ACCTGAGCTG |
| | GTGAAGCCTG |
| 101 | GGGCTTCAGT GAAGATATCC TGCAAGGCTT CTGGTTACTC |
| | ATTTACTGGC |
| 151 | TACTTTATGA ACTGGATGAG GCAGAGCCAT GGAAAGAGCC |
| | TTGAGTGGAT |
| 201 | TGGACGTATT AATCCTTACA ATGGTGATTC TTTCTACAAC |
| | CAGAAGTTCA |
| 251 | AGGACAAGGC CACATTGACT GTAGACAAAT CCTCTACCAC |
| | AGCCCACATG |
| 301 | GAGCTCCTGA GCCTGACATC TGAGGACTCT GCAGTCTATT |
| | ATTGTGGAAG |
| 351 | AGGATACGAC GCTATGGACT ACTGGGGTCA AGGAACCTCA |
| | GTCACCGTCT |
| 401 | CCTCA |

Shown below is the A1D5 mature light chain variable domain protein sequence, with CDRs underlined:

(SEQ ID NO: 55)

| | |
|---|---|
| 1 | DIQMTQTTSS LSASLGDRVT ISCRASQDIS NFLTWYQQKP |
| | DGTVKLLIYY |
| 51 | TSKLHSGVPS RFSGSGSGTD YSLTISNLEP GDIATYYCQQ |
| | VSKFPWTFGG |
| 101 | GAKLEIK |

The A1D5 Light Chain is a Murine Subgroup V Kappa Light Chain.

Shown below is the DNA sequence of the mature light chain variable domain (from pYL352), with its signal sequence underlined (light chain encoded signal is MVSTAQFLGLLLLCFQGTRC (SEQ ID NO: 56)):

(SEQ ID NO: 57)

| | |
|---|---|
| 1 | ATGGTGTCCA CAGCTCAGTT CCTTGGTCTC CTGTTGCTCT |
| | GTTTTCAAGG |
| 51 | TACCAGATGT GATATCCAGA TGACACAGAC TACATCCTCC |
| | CTGTCTGCCT |
| 101 | CTCTGGGAGA CAGAGTCACC ATTAGTTGCA GGGCAAGTCA |
| | GGACATTAGC |
| 151 | AATTTTTTAA CCTGGTATCA GCAGAAACCA GATGGAACTG |
| | TTAAACTCCT |
| 201 | GATCTACTAC ACATCAAAAT TACACTCAGG AGTCCCATCA |
| | AGGTTCAGTG |
| 251 | GCAGTGGGTC TGGGACAGAT TATTCTCTCA CCATTAGCAA |
| | CCTGGAACCG |
| 301 | GGTGATATTG CCACTTACTA TTGCCAACAG GTTAGTAAGT |
| | TTCCGTGGAC |
| 351 | GTTCGGTGGA GGCGCCAAGC TGGAAATCAA A |

mAbs LT101 and LT103

Antibodies LT101 (P1G4.4) and LT103 (P1G9.1) were found to be identical. Shown below is the LT101 and LT103 mature heavy chain variable domain protein sequence, with CDRs underlined:

(SEQ ID NO: 58)

| | |
|---|---|
| 1 | QVQLQQSGPE LVKPGASVQI SCKASGYVFS SSWMNWVKQR |
| | PGRGLEWIGR |
| 51 | IYPGDGDTDY TGKFKGKATL TADKSSNTAY MQLSSLTSVD |
| | SAVYFCASGY |
| 101 | FDFWGQGTPL TVSS |

### The Heavy Chain of Antibodies LT101 and LT103 are a Murine Subgroup II(B) Heavy Chain.

Shown below is the DNA sequence of the LT101 heavy chain variable domain (from pYL458 or pYL459), with its signal sequence underlined (heavy chain encoded signal is MGWSCIMFFLLSITAGVHC (SEQ ID NO: 59)):

(SEQ ID NO: 60)

| | |
|---|---|
| 1 | ATGGGATGGA GCTGTATCAT GTTCTTCCTC CTGTCAATAA |
| | CTGCAGGTGT |
| 51 | CCATTGCCAG GTCCAGCTGC AGCAGTCTGG ACCTGAGCTG |
| | GTGAAGCCTG |
| 101 | GGGCCTCAGT GCAGATTTCC TGCAAAGCTT CTGGCTACGT |
| | TTTCAGTAGT |
| 151 | TCTTGGATGA ACTGGGTGAA GCAGAGGCCT GGACGGGGTC |
| | TTGAGTGGAT |
| 201 | TGGGCGGATT TATCCTGGAG ATGGAGATAC TGACTACACT |
| | GGGAAGTTCA |
| 251 | AGGGCAAGGC CACACTGACT GCAGACAAAT CCTCCAACAC |
| | AGCCTACATG |
| 301 | CAGCTCAGCA GCCTGACCTC TGTGGACTCT GCGGTCTATT |
| | TCTGTGCAAG |
| 351 | TGGGTACTTT GACTTCTGGG GCCAAGGCAC CCCTCTCACC |
| | GTCTCCTCA |

Shown below is the LT101 and LT103 mature light chain variable domain protein sequence, with CDRs underlined:

(SEQ ID NO: 61)

| | |
|---|---|
| 1 | DITMTQSPSS MYASLGERVT ITCKASQDMN NYLRWFQQKP |
| | GKSPQTLIFR |
| 51 | ANRLVDGVPS RFSGSGSGQD YSLTISSLEF EDMGIYYCLQ |
| | HDKFPPTFGG |
| 101 | GTKLEIK |

### The Light Chain of LT101 and LT103 is a Murine Subgroup V Kappa Light Chain.

Shown below is the DNA sequence of the mature light chain variable domain (from pYL461 or pYL462), with its signal sequence underlined (light chain encoded signal is MRAPAQFLGILLLWFPGIKC (SEQ ID NO: 62)):

(SEQ ID NO: 63)

| | |
|---|---|
| 1 | ATGAGGGCCC CTGCTCAGTT TCTTGGCATC TTGTTGCTCT |
| | GGTTTCCAGG |
| 51 | TATCAAATGT GACATCACGA TGACCCAGTC TCCATCTTCC |
| | ATGTATGCAT |
| 101 | CTCTAGGAGA GAGAGTCACT ATCACTTGCA AGGCGAGTCA |
| | GGACATGAAT |
| 151 | AACTATTTAA GGTGGTTCCA GCAGAAACCA GGGAAGTCTC |
| | CTCAGACCCT |
| 201 | GATCTTTCGT GCAAACAGAT TGGTCGATGG GGTCCCATCA |
| | AGGTTCAGTG |
| 251 | GCAGTGGATC TGGGCAAGAT TATTCTCTCA CCATCAGCAG |
| | CCTGGAATTT |
| 301 | GAAGATATGG GAATTTATTA TTGTCTACAG CATGATAAAT |
| | TTCCTCCGAC |
| 351 | GTTCGGTGGA GGCACCAAGC TGGAAATCAA A |

### mAb LT102

Shown below is the LT102 (P1G8.2) mature heavy chain variable domain protein sequence, with CDRs underlined:

(SEQ ID NO: 64)

| | |
|---|---|
| 1 | EVKLVESGGG LVKPGGSLKL SCAVSGFTFS DYYMYWIRQT |
| | PEKRLEWVAT |
| 51 | IGDGTSYTHY PDSVQGRFTI SRDYATNNLY LQMTSLRSED |
| | TALYYCARDL |
| 101 | GTGPFAYWGQ GTLVTVSA |

### The LT102 Heavy Chain is a Murine Subgroup III(D) Heavy Chain.

Shown below is the DNA sequence of the LT102 heavy chain variable domain (from pYL375), with its signal sequence underlined (heavy chain encoded signal is MDFGLSWVFLVLVLKGVQC (SEQ ID NO: 65)):

(SEQ ID NO: 66)

| | |
|---|---|
| 1 | ATGGACTTCG GGTTGAGCTG GGTTTTCCTT GTCCTTGTTT |
| | TAAAAGGTGT |
| 51 | CCAGTGTGAA GTGAAGCTGG TGGAGTCTGG AGGAGGCTTA |
| | GTGAAGCCTG |
| 101 | GAGGGTCCCT GAAACTCTCC TGTGCAGTCT CTGGATTCAC |
| | TTTCAGTGAC |
| 151 | TATTATATGT ATTGGATTCG CCAGACTCCG GAAAAGCGGC |
| | TGGAGTGGGT |
| 201 | CGCAACCATT GGTGATGGTA CTAGTTACAC CCACTATCCA |
| | GACAGTGTGC |
| 251 | AGGGGCGATT CACCATCTCC AGAGACTATG CCACGAACAA |
| | CCTGTACCTG |
| 301 | CAAATGACTA GTCTGAGGTC TGAAGACACA GCCTTATATT |
| | ACTGTGCAAG |
| 351 | AGATCTTGGA ACCGGGCCTT TTGCTTACTG GGGCCAGGGG |
| | ACTCTGGTCA |
| 401 | CTGTCTCTGC A |

Shown below is the LT102 mature light chain variable domain protein sequence, with CDRs underlined:

(SEQ ID NO: 67)

| | |
|---|---|
| 1 | DVLMTQTPRS LPVSLGDQAS ISCRSSQNIV HSNGNTYLEW |
| | YLQKPGQSPK |
| 51 | LLIYKVSNRF SGVPDRFSGS GSGTDFTLKI SRVEAEDLGV |
| | YYCFQGSHFP |
| 101 | WTFGGGTKLE IK |

### The LT102 Light Chain is a Murine Subgroup II Kappa Light Chain.

Shown below is the DNA sequence of the mature light chain variable domain (from pYL378), with its signal sequence underlined (light chain encoded signal is MKLPVRLLVLMFWIPASSS (SEQ ID NO: 68)):

(SEQ ID NO: 69)

| | |
|---|---|
| 1 | ATGAAGTTGC CTGTTAGGCT GTTGGTGCTG ATGTTCTGGA |
| | TTCCTGCTTC |
| 51 | CAGCAGTGAC GTTTTGATGA CCCAAACTCC ACGCTCCCTG |
| | CCTGTCAGTC |
| 101 | TTGGAGATCA AGCCTCCATC TCTTGCAGAT CTAGTCAGAA |
| | CATTGTTCAT |
| 151 | AGTAATGGAA ACACCTATTT AGAATGGTAC CTGCAGAAAC |
| | CAGGCCAGTC |
| 201 | TCCAAAGCTC CTGATCTACA AAGTTTCCAA CCGATTTTCT |
| | GGGGTCCCAG |
| 251 | ACAGGTTCAG TGGCAGTGGA TCAGGGACAG ATTTCACACT |
| | CAAGATCAGC |
| 301 | AGAGTGGAGG CTGAGGATCT GGGAGTTTAT TACTGCTTTC |
| | AAGGTTCACA |
| 351 | TTTTCCTTGG ACATTCGGTG GAGGCACCAA GCTGGAGATC AAA |

### mAb LT105

Shown below is the LT105 (P2E9.7) mature heavy chain variable domain protein sequence, with CDRs underlined:

(SEQ ID NO: 70)

| | |
|---|---|
| 1 | DVQLQESGPG LVKPSQSLSL TCSVTGYSIT SGYYWNWIRQ |
| | FPGNKLEGMG |
| 51 | YISYDGSNNY NPSLKNRISI TRDSSKNQFF LKLNSVTAED |
| | SGTYYCARDA |
| 101 | YSYGMDYWGQ GTSVTVSS |

### The LT105 Heavy Chain is a Murine Subgroup I(A) Heavy Chain.

Shown below is the DNA sequence of the LT105 heavy chain variable domain (from pYL382), with its signal sequence underlined (heavy chain encoded signal is MMVLSLLYLLTAIPGILS (SEQ ID NO: 71)):

(SEQ ID NO: 72)

| | |
|---|---|
| 1 | ATGGACTTCG GGTTGAGCTG GGTTTTCCTT GTCCTTGTTT |
| | TAAAAGGTGT |
| 51 | CCAGTGTGAA GTGAAGCTGG TGGAGTCTGG AGGAGGCTTA |
| | GTGAAGCCTG |
| 101 | GAGGGTCCCT GAAACTCTCC TGTGCAGTCT CTGGATTCAC |
| | TTTCAGTGAC |
| 151 | TATTATATGT ATTGGATTCG CCAGACTCCG GAAAAGCGGC |
| | TGGAGTGGGT |
| 201 | CGCAACCATT GGTGATGGTA CTAGTTACAC CCACTATCCA |
| | GACAGTGTGC |
| 251 | AGGGGCGATT CACCATCTCC AGAGACTATG CCACGAACAA |
| | CCTGTACCTG |
| 301 | CAAATGACTA GTCTGAGGTC TGAAGACACA GCCTTATATT |
| | ACTGTGCAAG |
| 351 | AGATCTTGGA ACCGGGCCTT TTGCTTACTG GGGCCAGGGG |
| | ACTCTGGTCA |
| 401 | CTGTCTCTGC A |

Shown below is the LT105 mature light chain variable domain protein sequence, with CDRs underlined:

(SEQ ID NO: 73)

| | |
|---|---|
| 1 | DIVLTQSPAS LAVSLGQRAT ISCRASESVD NYGISFMHWY |
| | QQKPGQPPKL |
| 51 | LIYRASNLES GIPARFSGSG SRTDFTLTIN PVETDDVATF |
| | YCQQSNKDPY |
| 101 | TFGGGTKLEI K |

### The LT105 Light Chain is a Murine Subgroup III Kappa Light Chain.

Shown below is the DNA sequence of the mature light chain variable domain (from pYL383), with its signal sequence underlined (light chain encoded signal is METDTLLLWVLLLWVPGSTG (SEQ ID NO: 74)):

(SEQ ID NO: 75)

| | |
|---|---|
| 1 | ATGGAGACAG ACACACTCCT GCTATGGGTG CTGCTGCTCT |
| | GGGTTCCAGG |
| 51 | TTCCACAGGT GACATTGTGC TGACCCAATC TCCAGCTTCT |
| | TTGGCTGTGT |
| 101 | CTCTAGGGCA GAGGGCCACC ATCTCCTGCA GAGCCAGCGA |
| | AAGTGTTGAT |
| 151 | AATTATGGCA TTAGTTTTAT GCACTGGTAC CAGCAGAAAC |
| | CAGGACAGCC |
| 201 | ACCCAAACTC CTCATCTATC GTGCATCCAA CCTAGAATCT |
| | GGGATCCCTG |
| 251 | CCAGGTTCAG TGGCAGTGGG TCTAGGACAG ACTTCACCCT |
| | CACCATTAAT |
| 301 | CCTGTGGAGA CTGATGATGT TGCAACCTTT TACTGTCAGC |
| | AAAGTAATAA |
| 351 | GGATCCGTAC ACGTTCGGAG GGGGGACCAA GCTGGAAATA AAA |

### mAb LT107

Shown below is the LT107 (P5C4.1) mature heavy chain variable domain protein sequence, with CDRs underlined:

(SEQ ID NO: 76)

| | |
|---|---|
| 1 | QVQLKQSGPG LVQPSQNLSI TCTVSGFSLT NYGIHWIRQP |
| | PGKGLEWLGV |
| 51 | IWSGGSTDHN AAFISRLSIS KDNSKSQVFF TMNSLEVDDT |
| | AIYYCARNRA |
| 101 | YYRYEGGMDY WGQGTSVTVS S |

LT107 a murine subgroup I(B) heavy chain. Note the potential N-linked glycosylation site in FR1 that is shown in bold above.

Shown below is the DNA sequence of the LT107 heavy chain variable domain (from pYL447), with its signal sequence underlined (heavy chain encoded signal is MAVLGLLFCLVTFPSCVLS (SEQ ID NO: 77)):

(SEQ ID NO: 78)

| | |
|---|---|
| 1 | ATGGCTGTCC TGGGGCTGCT CTTCTGCCTG GTGACATTCC |
| | CAAGCTGTGT |
| 51 | CCTATCCCAG GTGCAGCTGA AACAGTCAGG ACCTGGCCTC |
| | GTGCAGCCCT |
| 101 | CACAGAACCT GTCCATCACC TGCACAGTCT CTGGTTTCTC |
| | ATTAACTAAC |
| 151 | TATGGTATAC ACTGGATTCG CCAGCCTCCA GGAAAGGGTC |
| | TGGAGTGGCT |
| 201 | GGGAGTGATA TGGAGTGGTG GAAGCACAGA CCATAATGCT |
| | GCTTTCATAT |
| 251 | CCAGACTGAG CATCAGCAAG GACAACTCCA AGAGCCAAGT |
| | TTTCTTTACA |
| 301 | ATGAACAGTC TGGAAGTTGA TGACACAGCC ATATACTACT |
| | GTGCCAGAAA |
| 351 | TAGAGCCTAC TATAGGTACG AGGGGGGTAT GGACTATTGG |
| | GGTCAAGGAA |
| 401 | CCTCAGTCAC CGTCTCCTCA |

Shown below is the LT107 mature light chain variable domain protein sequence, with CDRs underlined:

(SEQ ID NO: 79)

| | |
|---|---|
| 1 | DIKMTQSPSS MYASLGERVT ITCKASQDIN TYLNWFQQKP |
| | GKSPMTLIYR |
| 51 | ADRLLDGVPS RFSGSGSGQD YSLTISSLED EDMGIYYCQQ |
| | YDDFPLTFGA |
| 101 | GTKLELK |

This is a murine subgroup V kappa light chain. Shown below is the DNA sequence of the mature light chain variable domain (from pYL448), with its signal sequence underlined (light chain encoded signal is MVSSAQFLGILLLWFPGIKC (SEQ ID NO: 80)):

(SEQ ID NO: 81)

| | |
|---|---|
| 1 | ATGGTATCCT CAGCTCAGTT CCTTGGAATC TTGTTGCTCT |
| | GGTTTCCAGG |
| 51 | TATCAAATGT GACATCAAGA TGACCCAGTC TCCATCTTCC |
| | ATGTATGCAT |
| 101 | CTCTAGGAGA GAGAGTCACT ATCACTTGCA AGGCGAGTCA |
| | GGACATTAAT |
| 151 | ACCTATTTAA ACTGGTTCCA GCAGAAACCA GGGAAATCTC |
| | CTATGACCCT |
| 201 | GATCTATCGT GCAGACAGAT TGTTAGATGG GGTCCCATCA |
| | AGGTTCAGTG |
| 251 | GCAGTGGATC TGGGCAAGAT TATTCTCTCA CCATCAGCAG |
| | CCTGGAGGAT |
| 301 | GAGGATATGG GAATTTACTA TTGTCAACAG TATGATGACT |
| | TTCCTCTCAC |
| 351 | GTTCGGTGCT GGGACCAAGC TGGAGCTGAA A |

### mAb LT108

Shown below is the LT108 (P4F2.2) mature heavy chain variable domain protein sequence, with CDRs underlined:

(SEQ ID NO: 82)

| | |
|---|---|
| 1 | QVQLKQSGPG LVQPSQSLSI TCTVSGFSLT DYGIHWIRQP |
| | PGKGLEWLGV |
| 51 | IWSGGSTDHN AVFTSRLNIS KDNSKSQVFF KMNSLEPDDT |
| | AMYYCARNRA |
| 101 | YYRYEGGMDY WGQGTSVTVS S |

This is a murine subgroup I (B) heavy chain. Note the potential N-linked glycosylation site in FR3 that is shown in bold above. Shown below is the DNA sequence of the LT107 heavy chain variable domain (from pYL449), with its signal sequence underlined (heavy chain encoded signal is MAVLALLFCLVTFPSCVLS (SEQ ID NO: 83)):

(SEQ ID NO: 84)

| | |
|---|---|
| 1 | ATGGCTGTCT TAGCGCTGCT CTTCTGCCTG GTGACATTCC |
| | CAAGCTGTGT |
| 51 | CCTATCCCAG GTGCAGCTGA AGCAGTCAGG ACCTGGCCTC |
| | GTGCAGCCCT |
| 101 | CACAGAGCCT GTCCATCACC TGCACAGTCT CTGGTTTCTC |
| | ATTAACTGAC |
| 151 | TATGGTATAC ACTGGATTCG CCAGCCTCCA GGAAAGGGTC |
| | TGGAGTGGCT |
| 201 | GGGAGTGATA TGGAGTGGTG GAAGCACAGA CCATAATGCT |
| | GTCTTCACAT |
| 251 | CCAGACTGAA TATCAGCAAG GACAACTCCA AGAGTCAAGT |
| | TTTCTTTAAA |
| 301 | ATGAACAGTC TGGAACCTGA TGACACAGCC ATGTACTACT |
| | GTGCCAGAAA |
| 351 | TAGAGCCTAC TATAGGTACG AGGGGGGTAT GGACTACTGG |
| | GGTCAAGGAA |
| 401 | CCTCAGTCAC CGTCTCCTCA |

The heavy chains of LT107 and LT108 are 93.4% identical at the protein level, and IgBLAST analyses suggest that they were derived from similar V-D-J recombination events. Shown below is the alignment between LT107 (top-(SEQ ID NO: 76)) and LT108 (bottom-(SEQ ID NO: 82)) heavy chain variable domains:

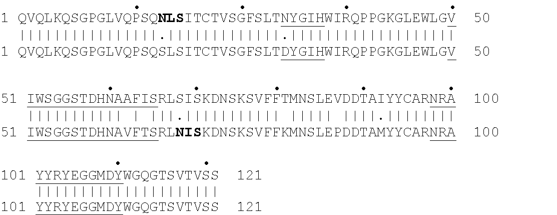

Shown below is the LT108 mature light chain variable domain protein sequence, with CDRs underlined:

(SEQ ID NO: 85)

| | |
|---|---|
| 1 | DIKMTQSPSS MYASLGERVT ITCKASQDIN TYLNWFQQKP |
| | GKSPMTLIYR |
| 51 | ADRLLDGVPS RFSGSGSGQD YSLTISSLED EDMGIYYCQQ |
| | YDDFPLTFGA |
| 101 | GTKLELK |

This is a murine subgroup V kappa light chain. At the protein level, it is 100% identical to the LT107 light chain. Shown below is the DNA sequence of the mature light chain variable domain (from pYL450), with its signal sequence underlined (light chain encoded signal is MVSSAQFLGILLLWFPGIKC (SEQ ID NO 86)):

(SEQ ID NO: 87)

| | |
|---|---|
| 1 | ATGGTATCCT CAGCTCAGTT CCTTGGAATC TTGTTGCTCT |
| | GGTTTCCAGG |
| 51 | TATCAAATGT GACATCAAGA TGACCCAGTC TCCATCTTCC |
| | ATGTATGCAT |
| 101 | CTCTAGGAGA GAGAGTCACT ATCACTTGCA AGGCGAGTCA |
| | GGACATTAAT |
| 151 | ACCTATTTAA ACTGGTTCCA GCAGAAACCA GGGAAATCTC |
| | CTATGACCCT |
| 201 | GATCTATCGT GCAGACAGAT TGTTAGATGG GGTCCCATCA |
| | AGGTTCAGTG |
| 251 | GCAGTGGATC TGGGCAAGAT TATTCTCTCA CCATCAGCAG |
| | CCTGGAGGAT |
| 301 | GAAGATATGG GAATTTACTA TTGTCAACAG TATGATGACT |
| | TTCCTCTCAC |
| 351 | GTTCGGTGCT GGGACCAAGC TGGAGCTGAA A |

It differs from the light chain of LT107 at a single nucleotide: a silent wobble position change in the codon for residue E81.
Below is the 9B4 mature heavy chain variable domain protein sequence, with CDRs underlined:

(SEQ ID NO: 88)

| | |
|---|---|
| 1 | QVTLKESGPG ILQPSQTLSL TCSFSGFSLS TSGMGVSWIR |
| | QPSGKGLEWL |
| 51 | AHIYWDDDKR YNPSLRSRLT ISKDTSRNQV FLKITSVDTA |
| | DTATYYCARR |
| 101 | EGYYGSSFDF DVWGAGTTVT VSS |

The heavy chain of antibody 9B4 is a murine subgroup I(B) heavy chain.

Shown below is the DNA sequence of the 9B4 heavy chain variable domain (from pYL573), with its signal sequence underlined (heavy chain encoded signal is MGRLTFSFLL LIVPAYVLS (SEQ ID NO: 89)):

(SEQ ID NO: 90)

| | |
|---|---|
| 1 | ATGGGCAGAC TTACATTCTC ATTCCTGCTG CTGATTGTCC |
| | CTGCATATGT |
| 51 | CCTTTCCCAG GTTACCCTGA AAGAGTCTGG CCCTGGGATA |
| | TTGCAGCCCT |
| 101 | CCCAGACCCT CAGTCTGACT TGTTCTTTCT CTGGGTTTTC |
| | ACTGAGCACT |
| 151 | TCTGGGATGG GTGTGAGCTG GATTCGTCAG CCTTCAGGAA |
| | AGGGTCTGGA |
| 201 | GTGGCTGGCA CACATTTACT GGGATGATGA CAAGCGCTAT |
| | AACCCATCCC |
| 251 | TGAGGAGCCG GCTCACAATC TCCAAGGATA CCTCCAGAAA |
| | CCAGGTATTC |
| 301 | CTCAAGATCA CCAGTGTGGA CACTGCAGAT ACTGCCACAT |
| | ACTACTGTGC |
| 351 | TCGAAGAGAG GGTTACTACG GTAGTAGCTT CGACTTCGAT |
| | GTCTGGGGCG |
| 401 | CAGGGACCAC GGTCACCGTC TCCTCT |

Shown below is the LT 9B4 mature light chain variable domain protein sequence, with CDRs underlined:

(SEQ ID NO: 91)

| | |
|---|---|
| 1 | QIVLSQSPAI LSASPGEKVT MTCRASSSVS YMIWYQQKPG |
| | SSPKPWIYAT |
| 51 | SSLASGVPTR FSGSGSGTSY SLTISRVEAA DAATYYCQQW |
| | SYNPLTFGAG |
| 101 | TKLELK |

This is a murine subgroup kappa VI kappa light chain. Shown below is the DNA sequence of the mature light chain variable domain (from pYL9B4), with its signal sequence underlined (light chain encoded signal is MDLQVQIFSFLLISASVKMSRG (SEQ ID NO: 92)):

(SEQ ID NO: 93)

| | |
|---|---|
| 1 | ATGGATTTAC AGGTGCAGAT TTTCAGCTTC CTGCTAATCA |
| | GTGCTTCAGT |
| 51 | CAAAATGTCC AGAGGACAAA TTGTTCTCTC CCAGTCTCCA |
| | GCAATCCTGT |
| 101 | CTGCATCTCC AGGGGAGAAG GTCACAATGA CTTGCAGGGC |
| | CAGCTCAAGT |
| 151 | GTGAGTTACA TGATCTGGTA CCAACAGAAG CCAGGATCCT |
| | CCCCCAAACC |
| 201 | CTGGATTTAT GCCACATCCA GCCTGGCTTC TGGAGTCCCT |
| | ACTCGCTTCA |
| 251 | GTGGCAGTGG GTCTGGGACC TCTTACTCTC TCACAATCAG |
| | CAGAGTGGAG |
| 301 | GCTGCAGATG CTGCCACTTA TTACTGCCAG CAGTGGAGTT |
| | ATAACCCGCT |
| 351 | CACGTTCGGT GCTGGGACCA AGCTGGAGCT GAAA |

### CDR Consensus Sequences

Sequence analysis of the various anti-LTα1β2 antibodies identified a number of consensus sequences within the CDRs. Table 1 describes the consensus sequences identified for the heavy chain sequences, and Table 2 describes the consensus sequences identified for the light chain sequences.

TABLE 1
Consensus sequences of heavy chain of anti-LT antibodies

| Antibody | | Antibody | |
|---|---|---|---|
| Designation | CDR1 | Designation | CDR2 |
| A0D9 | GFSLSTYGVH | A0D9 | VIWRGGNTNYNAAFMS (SEQ ID NO: 95) |
| | (SEQ ID NO: 94) | | |
| 108 | GFSLTDYGIH (SEQ ID | 108 | VIWSGGSTDHNAVFTS (SEQ ID NO: 98) |
| | NO: 97) | | |
| 107 | GFSLTNYGIH (SEQ ID | 107 | VIWSGGSTDHNAAFIS (SEQ ID NO: 101) |
| | NO: 100) | | |
| 9B4 | GFSLSTSGMGVS (SEQ | | |
| | ID NO: 103) | | |
| Consensus A | GFSLX₁X₂Y/SGX₃H/G | Consensus B | VIWX₁GGX₂TX₃X₄NAX₅FX₆S (SEQ ID NO: |
| | X₄ X₅ (SEQ ID NO: 105) | | 106) |
| | X₁ = S or T | | X₁ = R or S |
| | X₂ = T, D, or N | | X₂ = N or S |
| | X₃ = V, M, or I | | X₃ = N or D |
| | X₄ = absent or V | | X₄ = Y or H |
| | X₅ = absent or S | | X₅ = A or V |
| | (7/10 or 12 | | X₆ = M, T, or I |
| | identical) | | (10/16 identical) |
| A1D5 | GYSFTGYFMN (SEQ ID | A1D5 | RINPYNGDSFYNQKFKD (SEQ ID NO: |
| | NO: 109) | | 110) |
| 102 | GFTFSDYYMY | 102 | TIGDGTSYTHYPDSVQG (SEQ ID NO: 112) |
| | (SEQ ID NO: 111) | | |
| 101/103 | GYVFSSSWMN | 101/103 | RIYPGDGDTDYTGKFKG (SEQ ID NO: 115) |
| | (SEQ ID NO: 114) | | |
| 105 | GYSITSGYYWN | 105 | GYISYDGSNNYNPSLKN (SEQ ID NO: 118) |
| | (SEQ ID NO: 117) | | |
| | | 9B4 | HIYWDDDKRYNPS (SEQ ID NO: 119) |
| Consensus C | GX₁X₂X₃X₄X₅X₆X₇X₈X₉X₁₀ | Consensus D | X₁X₂X₃X₄X₅X₆X₇X₈X₉X₁₀YX₁₁X₁₂X₁₃X₁₄X₁₅X₁₆ |
| | (SEQ ID NO: 120) | | (SEQ ID NO: 15) |
| | X₁ = Y, F | | X₁ = R, T, G, or absent |
| | X₂ = S, T, or V | | X₂ = I, H |
| | X₃ = F or I | | or Y |
| | X₄ = T or S | | X₃ = N, G, Y, or I |
| | X₅ = G, D, or S | | X₄ = P, D, Y, or S |
| | X₆ = Y, S, or G | | X₅ = Y, W or G |
| | X₇ = F, Y, or W | | X₆ = N, T, or D |
| | X₈ = M or Y | | X₇ = G, D or S |
| | X₉ = N, Y or W | | X₈ = D, Y, or S |
| | X₁₀ = absent or N | | X₉ = S, T, K, or N |
| | | | X₁₀ = F, H, D, R, or N |
| | | | X₁₁ = N, P, or T |
| | | | X₁₂ = Q, D, G, or P |
| | | | X₁₃ = K or S |
| | | | X₁₄ = F, V, or L |
| | | | X₁₅ = K or Q |
| | | | X₁₆ = D, G, or N |

| Antibody | Antibody | |
|---|---|---|
| Designation | Designation | CDR3 |
| A0D9 | A0D9 | NQIYDGYYDYAMDY |
| | | (SEQ ID NO: 96) |
| 108 | 108 | NRAYYRYEGGMDY |
| | | (SEQ ID NO: 99) |
| 107 | 107 | NRAYYRYEGGMDY |
| | | (SEQ ID NO: 102) |
| 9B4 | 9B4 | REGYYGSSFDFDV |
| | | (SEQ ID NO: 104) |
| | Consensus E | G/AYYG/A |
| | | (SEQ ID NO: 16) |
| Consensus A | 105 | DAYSYGMDY |
| | | (SEQ ID NO: 107) |
| | A1D5 | GYDAMDY |
| | | (SEQ ID NO: 108) |
| A1D5 | | |
| 102 | 102 | GTGPFAY (SEQ ID NO: 113) |
| 101/103 | 101/103 | GYFDF (SEQ ID NO: 116) |
| 105 | | |
| Consensus C | | |

TABLE 2
Consensus sequences of light chain of anti-LT antibodies

| Antibody | | Antibody | | Antibody | |
|---|---|---|---|---|---|
| Designation | CDR1 | Designation | CDR2 | Designation | CDR3 |
| A0D9 | KASQDINTYLN | A0D9 | RANRLVD (SEQ ID NO: 122) | | |
| | (SEQ ID NO: 121) | | | | |
| 108 | KASQDINTYLN | 108 | RADRLLD (SEQ ID NO: 123) | 108 | QQYDDFPLT |
| | (SEQ ID NO: 121) | | | | (SEQ ID NO: 124) |
| 107 | KASQDINTYLN | 107 | RADRLLD (SEQ ID NO: 123) | 107 | QQYDDFPLT |
| | (SEQ ID NO: 121) | | | | (SEQ ID NO: 124) |
| A1D5 | RASQDISNFLT | 101/103 | RANRLVD (SEQ ID NO: 126) | A1D5 | QQVSKFPWT |
| | (SEQ ID NO: 125) | | | | (SEQ ID NO: 127) |
| 101/103 | KASQDMNNYLR | Consensus B | RAX₁RLX₂D (SEQ ID NO: 129) | 102 | FQGSHFPWT |
| | (SEQ ID NO: 128) | | X₁ = N or D | | (SEQ ID NO: 130) |
| | | | X₂ = V or L | | |
| | | | (5/7 identical) | | |
| Consensus A | X₁ASQDX₂X₃X₄X₅LX₆ | | | 105 | QQSNKDPYT |
| | (SEQ ID NO: 131) | | | | (SEQ ID NO: 132) |
| | X₁ = K or R | | | | |
| | X₂ = I or M | | | | |
| | X₃ = N or S | | | | |
| | X₄ = T or N | | | | |
| | X₅ = Y or F | | | | |
| | X₆ = N, T, or R | | | | |
| | (5/11 identical) | | | | |
| | | | | 9B4 | QQWSYNPLT |
| | | | | | (SEQ ID NO: 133) |
| | | | | Consensus C | X₁QX₂X₃X₄X₅PX₆T |
| | | | | | (SEQ ID NO: 18) |
| | | | | | X₁ = Q or F |
| | | | | | X₂ = Y, V, G, W, or S |
| | | | | | X₃ = D, S, or N |
| | | | | | X₄ = D, H, Y, or K |
| | | | | | X₅ = F, N |
| | | | | | or D |
| | | | | | X₆ = W, L, or Y |
| | | | | | (3/9 identical) |
| 105 | RASESVDNYGISFMH | A1D5 | YTSKLHS (SEQ ID NO: | A0D9 | LHYDAFPWT |
| | (SEQ ID NO: 134) | | 135) | | (SEQ ID NO: 136) |
| 9B4 | RASSSVSYMI | 102 | KVSNRFS (SEQ ID NO: | 101/103 | LQHDKFPPT |
| | (SEQ ID NO: 137) | | 138) | | (SEQ ID NO: 139) |
| Consensus F | RASX₁SVX₂X₃X₄X₅ | 105 | RASNLES | | |
| | (SEQ ID NO: 140) | | (SEQ ID NO: 141) | | |
| | X₁ = E or S | | | | |
| | X₂ = D or S | | | | |
| | X₃ = N or Y | | | | |
| | X₄ = Y or M | | | | |
| | X₅ = G or I | | | | |
| | | 105A | KASNLES (SEQ ID NO: | | |
| | | | 142) | | |
| | | 105B | RASSLES (SEQ ID NO: | | |
| | | | 143) | | |
| | | 105C | KASSLES (SEQ ID NO: | | |
| | | | 144) | | |
| | | 9B4 | ATSSLAS (SEQ ID NO: | | |
| | | | 145) | | |
| 102 | RSSQNIVHSNGNTYLE | | | | |
| | (SEQ ID NO: 146) | | | | |
| | | Consensus D | X₁X₂SX₃X₄X₅S (SEQ ID | Consensus E | LX₁X₂DX₃FPX₄T (SEQ ID |
| | | | NO: 147) | | NO: 148) |
| | | | X₁ = A, Y, R, or K | | X₁ = H or Q |
| | | | X₂ = T, A, or V | | X₂ = H or Y |
| | | | X₃ = K, S or N | | X₃ = A or K |
| | | | X₄ = L or R | | X₄ = W or P |
| | | | X₅ = H, E, A, or F | | (5/9 identical) |
| | | | (2/7 identical) | | |

### Example 2

### In Vitro Activity of Anti-Lymphotoxin (LT) Antibodies IL-8 Release Assay

The IL-8 release assay was used to determine the functional activity of the anti-LT antibodies described in Example 1. The IL-8 release assay is based on the secretion of IL-8, which is observed after soluble recombinant human lymphotoxin α1β2 binds to cell surface lymphotoxin beta receptor on A375 cells (human melanoma cell line). The IL-8 release assay measures the ability of an antibody to block this IL-8 secretion by binding to soluble lymphotoxin α1β2, preventing it from binding to the lymphotoxin beta receptor. The IL-8 that is secreted into the media supernatant is then measured with an ELISA assay.

The antibody was diluted to the appropriate concentrations and incubated with soluble recombinant human lymphotoxin α1β2 (170 ng/ml) for 1 hour at room temperature in a 96-well microtiter plate. The concentration of lymphotoxin α1β2 was optimized by titration experiments that determined the maximum amount of IL-8 release.

Fifteen to twenty thousand A375 cells were then added to each well, and the plate was incubated for 17 hours at 37° C. 5% CO₂ At the end of the incubation period, the plate was centrifuged and the supernatant was harvested. The supernatants were tested for IL-8 concentration with a standard sandwich ELISA assay. The IL-8 concentrations were plotted versus antibody concentrations, and an IC50 was determined from a 4-parameter curve fit of the data (see FIGS. 1A and 1B for inhibition curves). Table 3 describes the calculated IC50 values for each antibody. In calculating the IC50 values, the antibody concentration present during the pre-incubation step with LTα1β2 (rather than the concentration of antibody after addition of cells and buffer which was 4× lower).

TABLE 3
Summary of IC50 determinations for inhibition of IL-8 release and percent
inhibition of IL-8 release

| Antibody | IC50 nM | Maximum % Inhibition |
|---|---|---|
| 9B4 | 0.6 | 95 |
| 102 | 0.406; 0.991 | 92 |
| 103 | 1.11 | 90 |
| 105 | 0.52; 1.056 | 100 |
| 107 | 0.53 | 95 |
| 108 | 1.3 | 100 |
| A1D5 | 2.5 | 94 |
| A0D9 | 1.67 | 94 |
| C37 | — | No inhibition |
| B27 | — | No inhibition |
| B9 | Approximately 500 nM | 53% @667 nM |
| | (estimate) | |

### LT α3 ELISA

In addition, binding experiments revealed that of the anti-LT antibodies described in Example 1, only mAb LT101/LT103 bound LTα3 (soluble homotrimer), while the others did not. MAb LT101/LT103 was able to block the LTα1β2-LTBR interaction (about 70% maximum blockade) as measured using the assay below. However, LT101/103 could not block the interaction between LTα3 and TNFR-Ig (p55) (assessed in blocking elisa format).

For the LTa3 ELISA, microtiter plates were coated with LTα3 (1 or 5 ug/ml in PBS) then nonspecific binding sites were blocked with a 1% casein buffer. Samples (antibodies, receptor-Ig) were added and binding detected with HRP-conjugated anti-murine Ig antibodies. For assessment of ability of mAbs to block interaction between LTα3 and TNFR-hIg (p55), plates were coated with LTα3 and blocked as described above. Serial dilutions of antibodies were added to plate 30 minutes prior to TNFR-Ig addition. Binding of TNFR-hIg to plate-bound LTα3 was detected with an HPR conjugated anti-human Ig antibody.

### LTBR-Ig Blocking Assay (II-23 Assay)

II-23 cells were incubated with 50 ng/ml PMA for 4 hours at 37° C. 5% CO2. The cells were washed and 500,000 cells were added to each well of a 96-well plate. The antibody was diluted to the appropriate concentrations and added to the II-23 cells. After a 30 minute incubation period at 4° C., the biotin labeled LTβR-Ig is added to each well for a final concentration of 1 ug/ml. The cells are incubated at 4° C. for an additional 30 minutes, and then washed 3 times. Streptavidin-PE was diluted to 1/500 and added to each well and incubated for 1 hour at 4° C. The cells were washed once and read by FACS analysis. The mean fluorescence intensity is plotted versus antibody concentrations, and an IC50 is determined from a 4-parameter curve fit of the data.

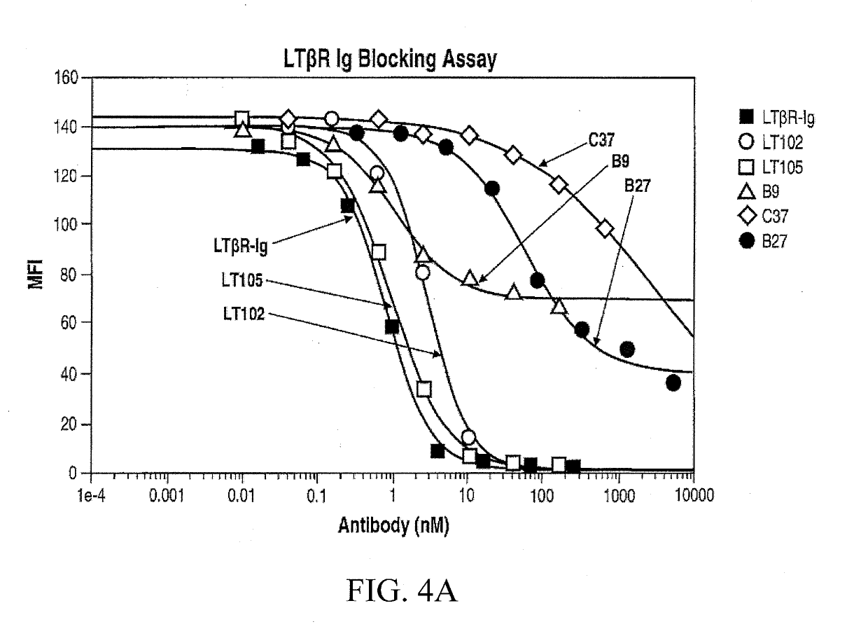

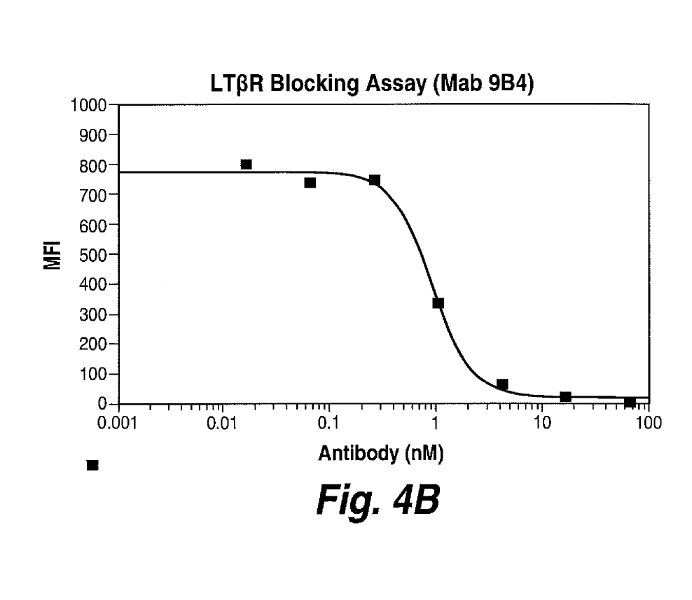

A number of mAbs identified had greater than 90% potency in an II-23 assay, including LT105, 9B4 LT102, A1.D5, and AOD9. mAbs LT102 and LT105 had greater than 98% blockade in an II-23 assay. As shown in FIG. 4, LT102 and LT105 exhibited superior potency in an II-23 blocking assay relative to anti-LT antibodies B9 (see U.S. Pat. No. 5,925,351), C37, and B27 (C37 and B27 are both described in Browning et al. (1995) J Immunol 154:33). A summary of the data are shown in Table 4:

TABLE 4
Maximum Percent Inhibition of LTβR binding to LT

| Antibody | Maximum % Inhibition |
|---|---|
| A0D9 | 92 |
| 105 | 97 |
| 9B4 | 99 |
| 103 | 77 |
| 102 | 98 |
| 107 | 80 |
| 108 | 81 |
| A1D5 | 92 |
| B9 | 44 |

### Cross-Reactivity

LT105, 9B4 and A1D5 also bound to LT from cynomolgus macaques (Macaca fascicularis) as did LT102 on a low plateau. A summary of the cross-reactivity assessment for some of the anti-LT mAbs is described below in Table 5. It is noteworthy that certain of the prior art antibodies did not bind to Cyno LT (e.g., B9).

TABLE 5

| mAb | A1D5 | LT102 | LT105 | 9B4 | LT107-9 | CE25 |
|---|---|---|---|---|---|---|
| Human LT | + | + | + | + | + | + (low |
| | | | | | | plateau) |
| Cyno LT | + | + (low | + | + | +/− | + (low |
| | | plateau) | | | | plateau) |

### Epitope Analysis

Cross-blocking experiments were performed to determine the epitopes bound by the new anti-LT antibodies described in Example 1. Cross-reactivity was also determined for anti-LT antibodies known in the art. Table 6 provides an overview of the cross-blocking study.

TABLE 6
Cross-blocking results

| | LT012 | LT105 | 9B4 | LT107 | A1D5 | A0D9 | B9 | C37 | B27 |
|---|---|---|---|---|---|---|---|---|---|
| LT102 | | − | − | − | − | − | − | − | − |
| LT105 | − | | + | − | − | − | − | − | − |
| 9B4 | | + | | | | | | | |
| LT107 | − | − | − | | − | + | − | − | − |
| A1D5 | − | − | − | − | | − | − | − | − |
| A0D9 | − | − | − | + | − | | − | − | − |
| B9 | − | − | − | − | − | − | | − | − |
| C37 | − | − | − | − | − | − | − | | + |
| B27 | − | − | − | − | − | − | − | + | |

As described in Table 6, there was limited cross-reactivity among the new anti-LT antibodies. Furthermore, LT102, LT105, 9B4, LT9B4, LT107, A1D5, A0D9 all bound epitopes distinct from anti-LT antibodies B9, C37, and B27.

As LT102 bound cyno LT with a lower plateau relative to human LT. This result suggested that critical contact point(s) for LT102 were likely in the non-homologous region between cyno and human LT. As such, variant forms of human LTβ were designed in this region based on molecular modelling, including the following amino acid substitutions: D151R/Q153R; R193A/R194A; D151R/Q153R/R193A/R194A; PLK(96, 97, 98)WMS; TTK(106, 107, 108)ASQ; TTK(106, 107, 108)AWQ; FA(231, 232)YR; T114R; DAE(121, 122, 123)PTH; and P172R.

The results showed that LTBR-Fc (positive control) at concentrations of both 100 ng/ml and 10 ng/ml, bound to all members of the mutant LT panel. Antibody LT102, however, bound to all members of the mutant LT panel (at the same concentrations as the LTBR-Fc positive control), except for mutants R193A/R194A and D151R/Q153R/R193A/R194A. Thus, residues R193 and R194 are critical for LT102 binding to human LT.

Antibody LT105 was found to bind to cyno LT but not murine LT. This result suggested that critical contact point(s) for LT105 were likely in the non-homologous region between cyno and murine LT. Mutant forms of human LT were designed within this region (based on the likelihood of interacting with LTBR). Variant forms of human LT were designed based on molecular modelling, including the following amino acid substitutions: D151R/Q153R; R193A/R194A; D151R/Q153R/R193A/R194A; PLK(96, 97, 98)WMS; TTK(106, 107, 108)ASQ; TTK(106, 107, 108)AWQ; FA(231, 232)YR; T114R; DAE(121, 122, 123)PTH; and P172R.

The results showed that LTBR-Fc (positive control) at concentrations of both 100 ng/ml and 10 ng/ml, bound to all members of the mutant LT panel. Antibody LT105, however, did not bind mutants PLK(96, 97, 98)WMS; TTK(106, 107, 108)ASQ; and TTK(106, 107, 108)AWQ. Thus, P96/L97/K98 and T106/T107/K108 were found to be critical to LT binding for LT105. 9B4 was found to cross compete with LT105 and its binding to be affected by the P96/L97/K98 mutations to LTβ, but not by mutations at positions 106, 107, or 108.

In conclusion, the epitope mapping of LT102, LT105, 9B4 and A1D5 using mutant forms of human LTβ showed that R193/R194 are critical for LT102 binding, and that P96/L97/K98 and T106/T107/K108 are critical residues for LT105 and 9B4 binding. Similar mutant studies revealed that residue P172 is critical to A1D5 binding to human LT, and that residues D151/Q153 are critical for LT107 and A0D9 binding.

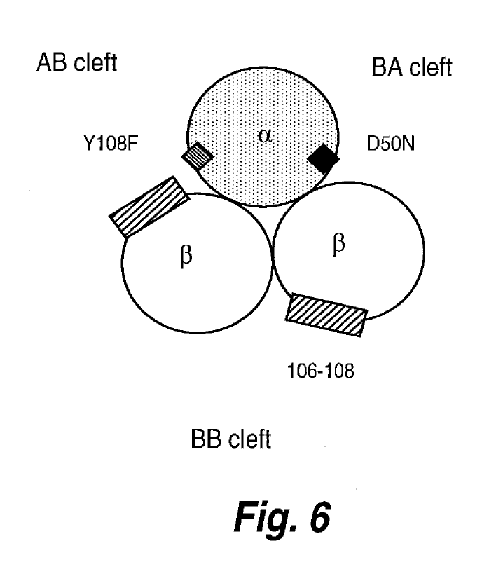

A schematic of the LT heterotrimer is described in FIG. 6. On subunit LTα, D50N and Y108F mutations define the sides of the αβ/βα clefts. In addition, LTB mutations that block LT105 binding align closely to the Y108F site.

### Example 3

### In Vivo Activity of Anti-Lymphotoxin (LT) Antibodies

The following materials and methods were used in this Example:

MICE: NOD-scid IL2rgnull pups (<72 hrs old) were irradiated (100 rads) and immediately received 3×10⁴ human CD34+ cord blood cells via RO sinus injection. For additional details, see Pearson et al. (2008) Curr Top Microbiol Immunol 324:25.

REAGENTS: LT102, LT105, and B9 are murine anti-human LTa1b2 (mIgG1) antibodies (BIIB, no cross to murine LT). BBF6 is a hamster anti-murine LTa1b2 antibody (BIIB, no cross to human LT). Murine LTBR-mIgG1 was used as a positive control for blockade of LT-LTBR interactions (shown to bind human LT with a ˜2× lower affinity than for murine LT). MOPC-21 is a murine IgG1 antibody used as an isotype control antibody.

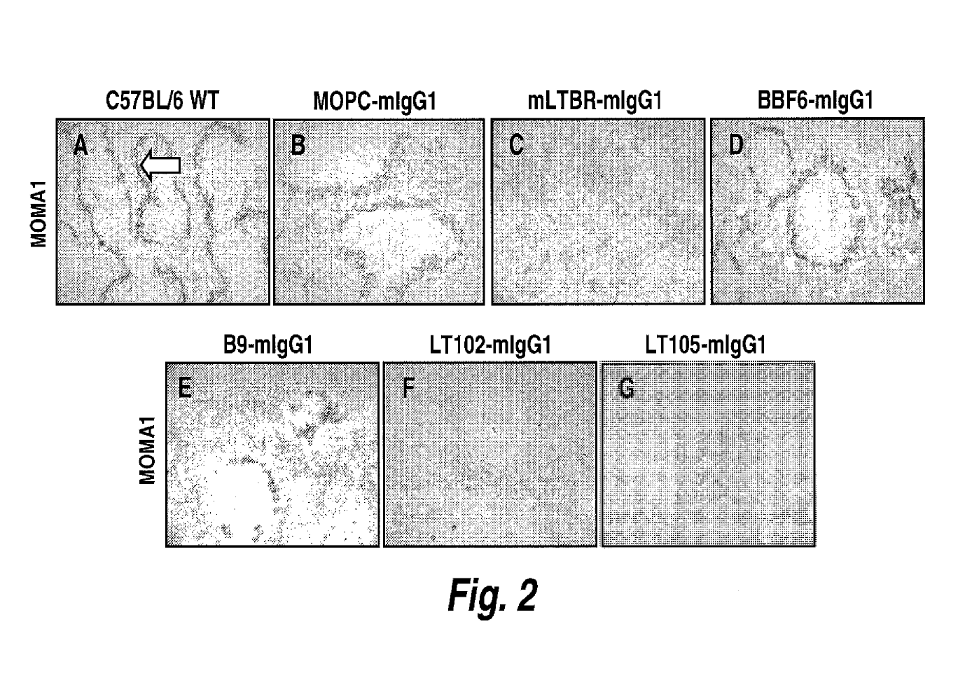

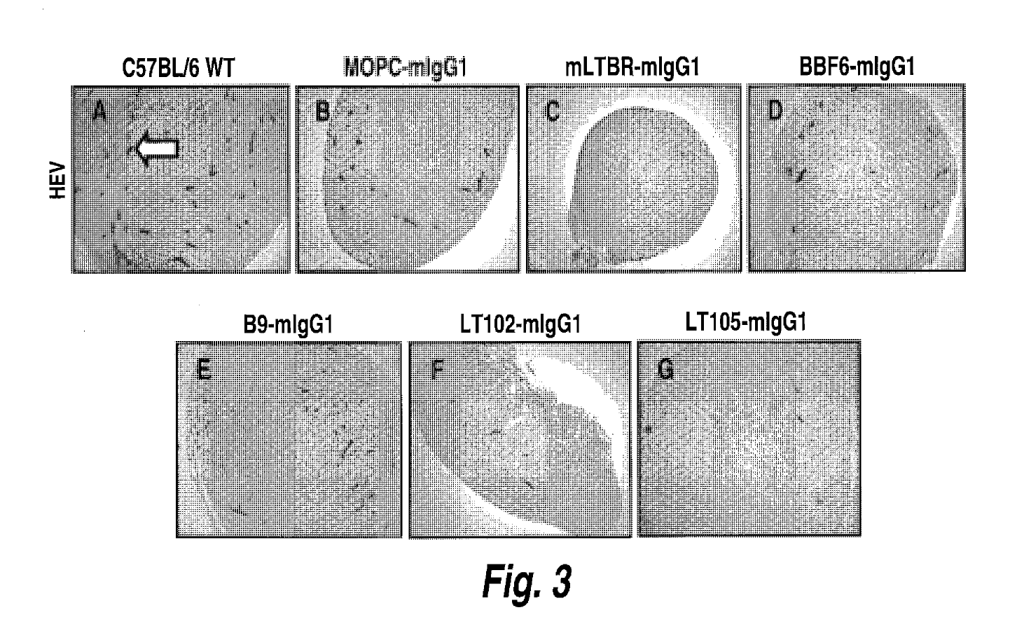

DOSING: At approximately 4 months of age, reconstituted mice were randomized into groups (n=5 mice/group). Mice were injected with either isotype control (MOPC-21), positive control (mLTBR-mIgG1), BBF6, B9, LT102 or LT105 at 50 ug/mouse/week (FIG. 2) or 200 ug/mouse/week (FIG. 3) (intraperitoneal administration, 5 injections total, n=5 mice/group). 7 days after the final injection, tissues were collected for analysis.

HISTOLOGICAL ANALYSES: PNAd/MECA79 (HEV): Lymph node tissue was fixed in 10% neutral buffered formalin for 24 hours and stored in paraffin blocks. 3 um sections were cut, deparaffinized and antigen retrival (Dako) was performed. Endogenous peroxidase block (Dako) and Fc block (rabbit serum) followed prior to application of rat anti-mouse PNAd primary antibody (1:300) (BD). A biotinylated rabbit anti-rat IgG (H+L) secondary antibody (Vector) and ABC Standard Kit (Vectastain) were used prior to development with DAB substrate (Vector). Mayer's hematoxylin (Sigma) nuclear counterstain was the final step before slides were serially dehydrated in 95% and 100% alcohol and stored with Permount coverslips.

Sialoadhesin/MOMA-1: 10 um sections were cut from spleen tissue frozen in OCT with methylbutane and stored at −80° C. Slides were fixed in acetone, rehydrated in 1×TBS and endogenous peroxidase block and Fc block (BSA) were performed. Sections were stained with a rat anti-mouse MOMA-1 FITC primary antibody (1:100) (Serotec). Anti-FITC-AP secondary antibody (Roche) was used prior to development with an AP Substrate Kit (Vector). Sections were covered using Crystal Mount and allowed to air dry at room temperature overnight.

To investigate the functional activity of the anti-human LTα1β2 mAbs, LT102 and LT105 with regard to the historical mAb, B9, NOD-scid IL2rynull mice engrafted with CD34+ human cord blood cells were used. These mice support the development of many components of a functional human immune system. In particular, chimeric mice have been successfully reconstituted and demonstrate MECA-79+ HEVs in peripheral lymph nodes and a sialoadhesin/MOMA-1+ ring of macrophages in the spleen. Such structures are LT-LTβR dependent and, thus, can be used as a readout of the activity of administered anti-LT antibodies

Chimerized (huSCID) mice injected with MOPC-21 have a splenic sialoadhesin/MOMA-1+ metallophilic macrophage ring similar to that observed in wild-type, C57BL/6 mice, evidenced by positive MOMA-1 staining (see FIGS. 2A and 2B). Histological analysis showed that blockage of human LTα1β2 resulted in loss of splenic MOMA-1+ metallophilic macrophages. Inhibition of LTβR by injecting huSCID mice with mLTβR-mIg resulted in a disappearance of MOMA-1+ metallophilic macrophages (see FIG. 2C). This was not recapitulated with huSCID mice injected with antibody BBF6, a blocking mAb to murine LT α1β2 (see FIG. 2D), confirming that the source of LT α1β2 is human. HuSCID mice injected with the new antibodies to human LT α1β2 (LT102 and LT105) also showed similar loss of MOMA-1 staining (see FIGS. 2F and 2G). Notably, treatment with the prior art anti-human LT antibody, B9, did not result in loss of the MOMA-1+ macrophage structure (FIG. 2E).

High endothelial venules (HEVs) are specialized structures that assist cell entry into the lymph nodes. Development and maintenance of these structures have been shown to depend on LTβR expression. Histological analysis showed HEVs could be reduced with the blockade of human LT α1β2. In the chimeric model, HEVs were similarly demonstrated to be present in wild type mice (C57BL/6) and huSCID mice injected with MOPC-21 (FIGS. 3A,B), although in reduced frequency, but similarly depend on LTBR signaling as they were lost with LTβR-Ig treatment (huSCID mice injected with mLTBR-mIgG1) (FIG. 3C). As expected, administration of an anti-murine LT α1β2 mAb (BBF6) to huSCID mice had no effect (FIG. 3D). Blockade of huLT α1β2 in huSCID mice injected with either LT102 or LT105 significantly reduced HEVs (FIGS. 3F and 3G) while treatment with the prior art antibody, B9, had minimal effect on the HEV structure (FIG. 3E)

In conclusion, it was shown that the new anti-human LT antibodies, LT102 and LT105, have functional in vivo activity, superior to the prior art mAb, B9, including on targets that are likely to be critical for treating human disease. This was evidenced by a decreased density of CD169+ (sialoadhesin/MOMA-1/Siglec-1) macrophages. This conclusion is also supported by a decreased density of HEV and functional PNAd/MAdCAM (disrupted trafficking to lymph nodes).

### Example 4

### Humanization of Anti-Lymphotoxin (LT) Antibody LT105

The sequences of the murine LT105 light and heavy chains are set forth below:

### Light Chain:

| (SEQ ID NO: 149) | | |
|---|---|---|
| 1 | DIVLTQSPAS LAVSLGQRAT ISCRASESVDNYGI SFMHWYQQKP GQPPKLLIYR | 50 |
| 51 | ASNLESGIPA RFSGSGSRTD FTLTINP VET DDVATFYCQQ SNKDPYTFGG | 100 |
| 101 | GTKLEIK | |

### Heavy Chain:

| (SEQ ID NO: 150) | | |
|---|---|---|
| 1 | DVQLQESGPG LVKPSQSLSL TCSVT [symbol] SGY YWNWIRQF PGNKLEGMGY | 50 |
| 51 | ISYDGSNNYN PSLKNRISIT RDSSKNQFFL K LNSVTAEDSGTY YCARDAYSYGM | 100a |
| 101 | DYWGQGTSVT VSS | |

Underline: Kabat CDR residues
Italic: Chotia CDR residues
Bold: Canonical residues
Numbering is according to the Kabat scheme throughout this example.

### Analysis of the Murine Variable Regions

The complementarity determining regions (CDRs) contain the residues most likely to bind antigen and must be retained in the reshaped antibody. CDRs are defined by sequence according to Kabat et al (1991). CDRs fall into canonical classes (Chothia et al, 1989) where key residues determine to a large extent the structural conformation of the CDR loop. These residues are almost always retained in the reshaped antibody. The CDRs of the heavy and light chain were classified into canonical classes as follows:

| Light Chain: | | Heavy Chain: | |
|---|---|---|---|
| L1: 15 residues | Class 4 | H1: 6 (+5 Chothia) residues | Class 2 |
| L2: 7 residues | Class 1 | H2: 16 residues | Class 1 |
| L3: 9 residues | Class 1 | H3: 9 residues | No canonical |
| | | | class |

The canonical residues important for these CDR classes are indicated in Table 4.

TABLE 4
Canonical Residues mAb LT105

| | | |
|---|---|---|
| L1 | Class 4 | 2(I) 25(A) 27b(V) 33(M) 71(F) |
| L2 | Class 1 | 48(I) 51(A) 52(S) 64(G) |
| L3 | Class 1 | 90(Q) 95(P) |
| H1 | Class 2 | 24(V) 26(G) 27(Y) 29(1) 34(W) 94(R) |
| H2 | Class 1 | 55(G) 71(R) |

| | |
|---|---|
| H3 | No Canonical Class |

The variable light and heavy chains were compared with the consensus (Kabat et al, 1991) and germline sequences (Matsuda et al, 1998, Brensing-Kuppers et al, 1997) for murine and human subgroups using BLAST program and compiled consensus and germline blast protein sequence databases.

The variable light chain is a member of murine subgroup Kappa 3 (89% identity in 111 amino acid overlap; CDR-L3 is 1 residue shorter than usual) and likely originated from murine mu21-5 germline (94% identity in 99 amino acid overlap), as shown below.

| mu21-5 | | | | |
|---|---|---|---|---|
| LT105: | 1 | DIVLTQSPASLAVSLGQRATISCRASESVDNYGISFMHWYQQKPGQPPKLLIYRASNLES | 60 | |
| | | DIVLTQSPASLAVSLGQRATISCRASESVD+YG SFMHWYQQKPGQPPKLLIYRASNLES | | |
| Mu21-5: | 1 | DIVLTQSPASLAVSLGQRATISCRASESVDSYGNSFMHWYQQKPGQPPKLLIYRASNLES | 60 | |
| LT015: | 61 | GIPARFSGSGSRTDFTLTINPVETDDVATFYCQQSNKDP | 99 | (SEQ ID NO: 151) |
| | | GIPARFSGSGSRTDFTLTINPVE DDVAT+YCQQSN+DP | | (SEQ ID NO: 152) |
| Mu21-5: | 61 | GIPARFSGSGSRTDFTLTINPVEADDVATYYCQQSNEDP | 99 | (SEQ ID NO: 153) |

The variable heavy chain is a member of murine subgroup Heavy 1A (81% identity in 117 amino acid overlap; CDR-H1 and CDR-H2 are each 1 residue shorter than usual) and likely originated from murine VH36-60 germline (81% identity in 97 amino acid overlap), as shown below.

| muVH36-60 | | | | |
|---|---|---|---|---|
| LT105: | 1 | DVQLQESGPGLVKPSQSLSLTCSVTGYSITSGYYWNWIRQFPGNKLEGMGYISYDGSNNY | 60 | |
| | | +VQLQESGP LVKPSQ+LSLTCSVTG SITS Y WNWIR+FPGNKLE MGYISY GS Y | | |
| muVH3-60: | 1 | EVQLQESGPSLVKPSQTLSLTCSVTGDSITSDY-WNWIRKFPGNKLEYMGYISYSGSTYY | 59 | |
| LT105: | 61 | NPSLKNRISITRDSSKNQFFLKLNSVTAEDSGTYYCAR | 98 | (SEQ ID NO: 154) |
| | | NPSLK+RISITRD+SKNQ++L+LNSVT+ED+ TYYCAR | | (SEQ ID NO: 155) |
| muVH3-60: | 60 | NPSLKSRISITRDTSKNQYYLQLNSVTSEDTATYYCAR | 97 | (SEQ ID NO: 156) |

The variable light chain corresponds to human subgroup Kappa 4 (67% identity in 111 amino acid overlap; CDR-L1 is 2 residues shorter than usual) and is the closest to human B3 germline (66% identity in 99 amino acid overlap), as shown below.

| huB3 | | | | |
|---|---|---|---|---|
| LT105: | 1 | DIVLTQSPASLAVSLGQRATISCRASESV--DNYGISFMHWYQQKPGQPPKLLIYRASNL | 58 | |
| | | DIV+TQSP SLAVSLG+RATI+C++S+SV + +++ WYQQKPGQPPKLLIY AS | | |
| huB3: | 1 | DIVMTQSPDSLAVSLGERATINCKSSQSVLYSSNNKNYLAWYQQKPGQPPKLLIYWASTR | 60 | |
| LT105: | 59 | ESGIPARFSGSGSRTDFTLTINPVETDDVATFYCQQSNKDP | 99 | (SEQ ID NO: 157) |
| | | ESG+P RFSGSGS TDFTLTI+ ++ +DVA +YCQQ P | | (SEQ ID NO: 158) |
| huB3: | 61 | ESGVPDRFSGSGSGTDFTLTISSLQAEDVAVYYCQQYYSTP | 101 | (SEQ ID NO: 159) |

The variable heavy chain corresponds to human subgroup Heavy 2 (69% identity in 114 amino acid overlap; CDR-H1 is 1 residue shorter than usual; CDR-H2 is 3 residues shorter than usual) and is the closest to human VH4-28 germline (68% identity in 98 amino acid overlap), as shown below.

| huVH4-28 | | | | |
|---|---|---|---|---|
| LT105: | 2 | VQLQESGPGLVKPSQSLSLTCSVTGYSITSGYYWNWIRQFPGNKLEGMGYISYDGSNNYN | 61 | |
| | | VQLQESGPGLVKPS +LSLTC+V+GYSI+S +W WIRQ PG LE +GYI Y GS YN | | |
| huVH4-28: | 2 | VQLQESGPGLVKPSDTLSLTCAVSGYSISSSNWWGWIRQPPGKGLEWIGYIYYSGSTYYN | 61 | |
| LT105: | 62 | PSLKNRISITRDSSKNQFFLKLNSVTAEDSGTYYCAR | 98 | (SEQ ID NO: 160) |
| | | PSLK+R++++ D+SKNQF LKL+SVTA D+ YYCAR | | (SEQ ID NO: 161) |
| huVH4-28: | 62 | PSLKSRVTMSVDTSKNQFSLKLSSVTAVDTAVYYCAR | 98 | (SEQ ID NO: 162) |

### Modeling the Structure of the Variable Regions

For this humanization the model of LT105 variable regions was built based on the crystal structure PDB ID 2F58 for the light and heavy chains, using Modeler, SCWRL sidechain placement, and brief minimization in vacuum with the Gromos96 43b1 parameter set. 2F58 and LT105 have CDRs and framework regions of equal lengths.

### Analysis of the Reshaped Variable Regions

To choose antibody acceptor framework sequences for the light and heavy chains, candidates were identified having high similarity to the murine LT105 sequences in canonical, interface and veneer zone residues; the same length CDRs if possible (except CDR-H3); a minimum number of backmutations (i.e., changes of framework residue types from that of the human acceptor to that of the LT105 mature murine antibody). Human germline sequences filled in with human consensus residues in the FR4 framework region were considered as well.

### Frameworks Chosen:

Human germline sequence huL6 (with consensus human KV3 FR4) and human gil3004688 were selected from multiple candidates as the acceptor frameworks for light and heavy chains respectively (see sequences described below). Acceptor frameworks that were more distant from stable KV3 and HV3 consensus classes were chosen in order to improve the physico-chemical properties of humanized designs.

>LT105L
(SEQ ID NO: 149)

DIVLTQSPASLAVSLGQRATISCRASESVDNYGISFMHWYQQKPGQPPKLLIYRASNLESGIPARFSGSG
SRTDFTLTINPVETDDVATFYCQQSNKDPYTFGGGTKLEIK
>huL6

(SEQ ID NO: 163)

EIVLTQSPATLSLSPGERATLSCRASQSVSSYLAWYQQKPGQAPRLLIYDASNRATGIPARFSGSG
SGTDFTLTISSLEPEDFAVYYC
> Consensus human KV3 FR4 region

(SEQ ID NO: 181)
----------------------------------------------------------------------
-------------------------------FGQGTKVEIK
>LT105H
(SEQ ID NO: 150)
DVQLQESGPGLVKPSQSLSLTCSVTGYSITSGYYWNWIRQFPGNKLEGMGYIS YDGSNNYNPSLKNRISI
TRDSSKNQFFLKLNSVTAEDSGTYYCARDAYSYGMDYWGQGTSVTVSS
>gi|3004688
(SEQ ID NO: 164)
EVQLVESGGGLVQPGGSLRLSCAASGFTFSNYEMNWVRQAPGKGLEWISYISNGDNTIYYADSVKGRFTI
SRDSAKNSLYLHMHSLRAEDTAVYYCARGDYGGNGYFYYYAMDVWGQGTTVTVSS

CDRs, including Chothia definition, are underlined.

### Humanization Designs for LT105

The three different versions of the humanized LT105 light chain are described below The humanized light chain of LT105 included: Germline huL6 framework//consensus human KV4 FR4//LT105 L CDRs. Backmutations described below in L1, L2, and L3 are in lowercase, bold font. CDRs, including Chothia definition, are underlined.

> L0 = graft
(SEQ ID NO: 20)
EIVLTQSPATLSLSPGERATLSCRASESVDNYGISFMHWYQQKPGQAPRLLIYRASNLESGIPARFSGSGSGTDF
TLTISSLEPEDFAVYYCQQSNKDPYTFGQGTKVEIK
> L1
(SEQ ID NO: 21)
dIVLTQSPATLSLSPGERATLSCRASESVDNYGISFMHWYQQKPGQAPRLLIYRASNLESGIPARFSGSGSGTDF
TLTISSLEPEDFAVYYCQQSNKDPYTFGQGTKVEIK
> L2
(SEQ ID NO: 22)
dIVLTQSPATLSLSPGERATiSCRASESVDNYGISFMHWYQQKPGQAPRLLIYRASNLESGIPARFSGSGSGTDF
TLTISSLEPEDFAVfYCQQSNKDPYTFGQGTKVEIK
> L3
(SEQ ID NO: 23)
dIVLTQSPATLSLSPGERATiSCRASESVDNYGISFMHWYQQKPGQAPRLLIYRASNLESGIPARFSGSGSrTDF
TLTISSLEPEDFAVfYCQQSNKDPYTFGQGTKVEIK

The four different versions of the humanized LT105 heavy chain are described below The humanized heavy chain of LT105 included: gil3004688 framework//LT105 H CDRs. Backmutations described below in H1, H2, H3, and H4 are in lowercase, bold font. CDRs, including Chothia definition, are underlined.

> H0 = graft

(SEQ ID NO: 24)

EVQLVESGGGLVQPGGSLRLSCAASGYSITSGYYWNWVRQAPGKG
LEWISYISYDGSNNYNPSLKNRFTISRDSAKNSLYLHMHSLRAEDTAVY
YCARDAYSYGMDYWGQGTTVTVSS
> H1

(SEQ ID NO: 25)

EVQLVESGGGLVQPGGSLRLSCAvSGYSITSGYYWNWVRQAPGKG
LEgISYISYDGSNNYNPSLKNRFTISRDSAKNSfYLHMHSLRAEDTAVY
YCARDAYSYGMDYWGQGTTVTVSS
> H2

(SEQ ID NO: 26)

EVQLVESGGGLVQPGGSLRLSCAvSGYSITSGYYWNWiRQAPGKG
LEgIgYISYDGSNNYNPSLKNRiTISRDSAKNSfYLHMHSLRAEDTAVY
YCARDAYSYGMDYWGQGTTVTVSS
> H3

(SEQ ID NO: 27)

dVQLVESGGGLVQPGGSLRLSCAvtGYSITSGYYWNWiRQAPGKG
LEgIgYISYDGSNNYNPSLKNRiTISRDSAKNSfYLHMHSLRAEDTAVY
YCARDAYSYGMDYWGQGTTVTVSS
> H4

(SEQ ID NO: 28)

dVQLVESGGGLVQPGGSLRLSCAvtGYSITSGYYWNWiRQAPGKG
LEgmgYISYDGSNNYNPSLKNRiTISRDSAKNSfYLHlHSLRAEDTAVY
YCARDAYSYGMDYWGQGTTVTVSS

### Example 5

### Humanization of Anti-Lymphotoxin (Lt) Antibody LT102

The sequences of the murine LT102 light and heavy chains are set forth below:

### Light Chain:

| (SEQ ID NO: 165) | | |
|---|---|---|
| 1 | DVLMTQTPRS LPVSLGDQAS ISCRSSQNIVHSNGN TYLEWYLQKP GQSPKLLIYK | 50 |
| 51 | VSNRFSGVPD RFSGSGSGTD FTLKISR VEA EDLGVYYCFQ GSHFPWTFGG | 100 |
| 101 | GTKLEIK | |

### Heavy Chain:

| (SEQ ID NO: 166) | | |
|---|---|---|
| 1 | EV KLVESGGG LVKPGGSLKL SCAVS [symbol] DY YMYWIRQT PEKRLEWVAT | 50 |
| 51 | IGDGTSYTHYP DSVQGRFTIS RDYATNNLYL QMTSLRSEDTALY YCARDLGTGPF | 100a |
| 101 | AY WGQGTLVT VSA | |

Underline: Kabat CDR residues
Italic: Chotia CDR residues
Bold: Canonical residues
Numbering is according to the Kabat scheme throughout this example.

### Analysis of the Murine Variable Regions

The complementarity determining regions (CDRs) contain the residues most likely to bind antigen and must be retained in the reshaped antibody. CDRs are defined by sequence according to Kabat et al (1991). CDRs fall into canonical classes (Chothia et al, 1989) where key residues determine to a large extent the structural conformation of the CDR loop. These residues are almost always retained in the reshaped antibody. The CDRs of the heavy and light chain were classified into canonical classes as follows:

| | |
|---|---|
| Light Chain: | Heavy Chain: |

| | | | |
|---|---|---|---|
| L1: 16 residues | Class 4 | H1: 5 residues | Class 1 |
| L2: 7 residues | Class 1 | H2: 17 residues | Class 3 |
| L3: 9 residues | Class 1 | H3: 9 residues | Nocanonical |
| | | | class |

The canonical residues important for these CDR classes are indicated in Table 1.

TABLE 5

| | | |
|---|---|---|
| L1 | Class 4 | Ap2(V) 25(S) 27b(I) 33(L) 71(F) |
| L2 | Class 1 | 48(1) 51(V [atypical]) 52(S) 64(G) |
| L3 | Class 1 | 90(Q) 95(P) |
| H1 | Class 1 | 24(V) 26(G) 27(F) 29(F) 34(M) 94(R) |
| H2 | Class 3 | 54(T [atypical]) 71(R) |

| | |
|---|---|
| H3 | No Canonical Class |

The variable light and heavy chains were compared with the consensus (Kabat et al, 1991) and germline sequences (Matsuda et al, 1998, Brensing-Kuppers et al, 1997) for murine and human subgroups using BLAST program and to query a database comprising consensus and germline sequences. CDRs were excluded from the sequences for comparisons to germline.

The variable light chain of LT102 is a member of murine subgroup Kappa 2 (94% identity in 112 amino acid overlap) and likely originated from murine mucr1 germline (97% identity in 100 amino acid overlap). A comparison between the VL of LT102 and mucr1 is shown below.

| mucr1 | | | | |
|---|---|---|---|---|
| Query: | 1 | DVLMTQTPRSLPVSLGDQASISCRSSQNIVHSNGNTYLEWYLQKPGQSPKLLIYKVSNRF | 60 | |
| | | DVLMTQTP SLPVSLGDQASISCRSSQ+IVHSNGNTYLEWYLQKPGQSPKLLIYKVSNRF | | |
| Sbjct: | 1 | DVLMTQTPLSLPVSLGDQASISCRSSQSIVHSNGNTYLEWYLQKPGQSPKLLIYKVSNRF | 60 | |
| Query: | 61 | SGVPDRFSGSGSGTDFTLKISRVEAEDLGVYYCFQGSHFP | 100 | (SEQ ID NO: 167) |
| | | SGVPDRFSGSGSGTDFTLKISRVEAEDLGVYYCFQGSH P | | (SEQ ID NO: 168) |
| Sbjct: | 61 | SGVPDRFSGSGSGTDFTLKISRVEAEDLGVYYCFQGSHVP | 100 | (SEQ ID NO: 169) |

The variable heavy chain is a member of murine subgroup Heavy 3D (80% identity in 118 amino acid overlap) and likely originated from murine VH37.1 germline (86% identity in 98 amino acid overlap). A comparison between the VH of LT102 and VH37.1 is shown below.

| muVH37.1 | | | | |
|---|---|---|---|---|
| Query: | 1 | EVKLVESGGGLVKPGGSLKLSCAVSGFTFSDYYMYWIRQTPEKRLEWVATIGDGTSYTHY | 60 | |
| | | EVKLVESGGGLVKPGGSLKLSCA SGFTFS Y M W+RQTPEKRLEWVATI G SYT+Y | | |
| Sbjct: | 1 | EVKLVESGGGLVKPGGSLKLSCAASGFTFSSYGMSWVRQTPEKRLEWVATISGGGSYTYY | 60 | |
| Query: | 61 | PDSVQGRFTISRDYATNNLYLQMTSLRSEDTALYYCAR | 98 | (SEQ ID NO: 170) |
| | | PDSV+GRFTISRD A NNLYLQM+SLRSEDTALYYCAR | | (SEQ ID NO: 171) |
| Sbjct: | 61 | PDSVKGRFTISRDNAKNNLYLQMSSLRSEDTALYYCAR | 98 | (SEQ ID NO: 172) |

The variable light chain corresponds to human subgroup Kappa 2 (77% identity in 112 amino acid overlap) and is the closest to human A3 germline (76% identity in 100 amino acid overlap). A comparison of the VL of LT102 and huA3 is shown below.

| >huA3 | | | | |
|---|---|---|---|---|
| Query: | 1 | DVLMTQTPRSLPVSLGDQASISCRSSQNIVHSNGNTYLEWYLQKPGQSPKLLIYKVSNRF | 60 | |
| | | D++MTQ+P SLPV+ G+ ASISCRSSQ+++HSNG YL+WYLQKPGQSP+LLIY SNR | | |
| Sbjct: | 1 | DIVMTQSPLSLPVTPGEPASISCRSSQSLLHSNGYNYLDWYLQKPGQSPQLLIYLGSNRA | 60 | |
| Query: | 61 | SGVPDRFSGSGSGTDFTLKISRVEAEDLGVYYCFQGSHFP | 100 | (SEQ ID NO: 167) |
| | | SGVPDRFSGSGSGTDFTLKISRVEAED+GVYYC Q P | | (SEQ ID NO: 173) |
| Sbjct: | 61 | SGVPDRFSGSGSGTDFTLKISRVEAEDVGVYYCMQALQTP | 100 | (SEQ ID NO: 174) |

The variable heavy chain corresponds to human subgroup Heavy 3 (72% identity in 117 amino acid overlap) and is the closest to human VH3-21 germline (73% identity in 98 amino acid overlap). A comparison of the VH of LT102 and huVH3-21 is shown below.

| >huVH3-21 | | | | |
|---|---|---|---|---|
| Query: | 1 | EVKLVESGGGLVKPGGSLKLSCAVSGFTFSDYYMYWIRQTPEKRLEWVATIGDGTSYTHY | 60 | |
| | | EV+LVESGGGLVKPGGSL+LSCA SGFTFS Y M W+RQ P K LEWV++I +SY +Y | | |
| Sbjct: | 1 | EVQLVESGGGLVKPGGSLRLSCAASGFTFSSYSMNWVRQAPGKGLEWVSSISSSSSYIYY | 60 | |
| Query: | 61 | PDSVQGRFTISRDYATNNLYLQMTSLRSEDTALYYCAR | 98 | (SEQ ID NO: 170) |
| | | DSV+GRFTISRD A N+LYLQM SLR+EDTA+YYCAR | | (SEQ ID NO: 175) |
| Sbjct: | 61 | ADSVKGRFTISRDNAKNSLYLQMNSLRAEDTAVYYCAR | 98 | (SEQ ID NO: 176) |

### Modeling the Structure of the Variable Regions

For the humanization of LT102, a model of the LT102 variable regions was built based on the crystal structure PDB ID 1CLZ for the light and heavy chains, using Modeler, SCWRL sidechain placement, and brief minimization in vacuum with the Gromos96 43b1 parameter set. 1CLZ has 1 extra residue in CDR-H3.

### Analysis of the Reshaped Variable Regions

### Method:

To choose antibody acceptor framework sequences for the light and heavy chains, we used an antibody sequence database and query tools to identify suitable templates with the highest similarity to the murine LT102 sequences in canonical, interface and veneer zone residues; the same length CDRs (except CDR-H3); a minimum number of backmutations (i.e., changes of framework residue types from that of the human acceptor to that of the LT102 mature murine antibody); and no backmutations at all in the positions (L 4, 38, 43, 44, 58, 62, 65-69, 73, 85, 98 and H 2, 4, 36, 39, 43, 45, 69, 70, 74, 92) (see Carter and Presta, 2000). Human germline sequences filled in with human consensus residues in the FR4 region were considered as well.

### Frameworks Chosen:

Human germline sequence huA3 (with consensus HUMKV2 FR4) and human germline sequence huVH3-11 (with consensus HUMHV3 FR4) were selected from multiple candidates as the acceptor frameworks for light and heavy chains respectively. Sequences are described below.

>LT102L

(SEQ ID NO: 165)

DVLMTQTPRSLPVSLGDQASISCRSSQNIVHSNGNTYLEWYLQKPGQS
PKLLIYKVSNRFSGVPDRFSGSGSGTDFTLKISRVEAEDLGVYYCFQGS
HFPWTFGGGTKLEIK
>huA3

(SEQ ID NO: 177)

DIVMTQSPLSLPVTPGEPASISCRSSQSLLHSNGYNYLDWYLQKPGQSP
QLLIYLGSNRASGVPDRFSGSGSGTDFTLKISRVEAEDVGVYYC
> Consensus human KV2 FR4 region

(SEQ ID NO: 178)

----------------------------------------------
-----------------------------------------------
---------FGQGTKVEIK
>LT102H

(SEQ ID NO: 166)

EVKLVESGGGLVKPGGSLKLSCAVSGFTFSDYYMYWIRQTPEKRLEWV
ATIGDGTSYTHYPDSVQGRFTISRDYATNNLYLQMTSLRSEDTALYYCAR
DLGTGPFAYWGQGTLVTVSA
>huVH3-11

(SEQ ID NO: 179)

QVQLVESGGGLVKPGGSLRLSCAASGFTFSDYYMSWIRQAPGKGLEWV
SYISSSGSTIYYADSVKGRFTISRDNAKNSLYLQMNSLRAEDTAVYYCAR
> Consensus human HV3 FR4 region

(SEQ ID NO: 180)

------------------------------------------------
------------------------------------------------
-----------WGQGTLVTVSS

### CDRs, Including Chothia Definition, are Underlined.

For this antibody, not all canonical residue backmutations could be avoided: germline huA3 differs from LT102 L at 3 canonical residues (L 2, 27b, 51), and germline huVH3-11 differs from LT102 H at 1 canonical residue (H 24).

One version of the variable light reshaped chain was designed, and four versions of the variable heavy reshaped chain was designed, in addition to the light and heavy CDR graft sequences. For the heavy chain, the first version contains the fewest backmutations and the next versions contain more backmutations (i.e. they are the least “humanized”). The murine A113 was substituted by 5113 (present in human HV FR4) in all versions of the heavy chain, and was not analyzed as a backmutation. Numbering is according to the Kabat scheme.

### Backmutations in Reshaped VL

The reshaped light chain of humanized LT102 (huLT102) included a germline huA3 framework, consensus human KV2 FR4, and LT102 L CDRs. The backmutation in the light chain of hu102 included: 12V. V2 is a canonical residue supporting CDR-L1.

### Backmutations in Reshaped VH

The four versions of the reshaped heavy chain of humanized LT102 (huLT012) each included germline huVH3-11 framework, consensus human HV3 FR4, and LT102 H CDRs.

### Humanization Designs for LT102

The humanized LT102 light chain sequence is described below (for details regarding backmutation see above). The humanized light chain of LT102 included: Germline huA3 framework//consensus human KV2 FR4//LT102 L CDRs. Backmutations are in lowercase bold font. CDRs, including Chothia definition, are underlined.

> L0 = graft

(SEQ ID NO: 39)

DIVMTQSPLSLPVTPGEPASISCRSSQNIVHSNGNTYLEWYLQKPGQSPQ
LLIYKVSNRFSGVPDRFSGSGSGTDFTLKISRVEAEDVGVYYCFQGSHFP
WTFGQGTKVEIK
> L1

(SEQ ID NO: 40)

DvVMTQSPLSLPVTPGEPASISCRSSQNIVHSNGNTYLEWYLQKPGQSPQ
LLIYKVSNRFSGVPDRFSGSGSGTDFTLKISRVEAEDVGVYYCFQGSHFP
WTFGQGTKVEIK

The four different versions of the humanized LT102 heavy chain are described below The humanized heavy chain of LT102 included: Germline huVH3-11 framework//consensus human HV3 FR4//LT102 H CDRs. Backmutations described below in H1, H2, H3, and H4 are in lowercase, bold font. CDRs, including Chothia definition, are underlined.

> H0 = graft

(SEQ ID NO: 41)

QVQLVESGGGLVKPGGSLRLSCAASGFTFSDYYMYWIRQAPGKGLEWVS
TIGDGTSYTHYPDSVQGRFTISRDNAKNSLYLQMNSLRAEDTAVYYCAR
DLGTGPFAYWGQGTLVTVSS
> H1

(SEQ ID NO: 42)

QVQLVESGGGLVKPGGSLRLSCAvSGFTFSDYYMYWIRQAPGKGLEWVS
TIGDGTSYTHYPDSVQGRFTISRDNAKNSLYLQMNSLRAEDTAVYYCAR
DLGTGPFAYWGQGTLVTVSS
> H2

(SEQ ID NO: 43)

eVQLVESGGGLVKPGGSLRLSCAvSGFTFSDYYMYWIRQAPGKGLEWVS
TIGDGTSYTHYPDSVQGRFTISRDyAKNSLYLQMNSLRAEDTAVYYCAR
DLGTGPFAYWGQGTLVTVSS
> H3

(SEQ ID NO: 44)

eVkLVESGGGLVKPGGSLRLSCAvSGFTFSDYYMYWIRQAPGKGLEWVS
TIGDGTSYTHYPDSVQGRFTISRDyAKNSLYLQMNSLRAEDTAVYYCAR
DLGTGPFAYWGQGTLVTVSS
> H4

(SEQ ID NO: 45)

eVkLVESGGGLVKPGGSLRLSCAvSGFTFSDYYMYWIRQAPGKGLEWVS
TIGDGTSYTHYPDSVQGRFTISRDyAtNnLYLQMNSLRAEDTAVYYCAR
DLGTGPFAYWGQGTLVTVSS

### Example 6

### Alterations to Improved Solubility

The L0 H1 (Light chain of the 105 antibody version 0/heavy chain of the 105 antibody version 1) combination of humanized 105 light and heavy chains was chosen for expression and stability studies:

L0

(SEQ ID NO: 29)

| | |
|---|---|
| 1 | EIVLTQSPAT LSLSPGERAT LSCRASESVD NYGISFMHWY |
| | QQKPGQAPRL |
| 51 | LIYRASNLES GIPARFSGSG SGTDFTLTIS SLEPEDFAVY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

H1

(SEQ ID NO: 30)

| | |
|---|---|
| 1 | EVQLVESGGG LVQPGGSLRL SCAVSGYSIT SGYYWNWVRQ |
| | APGKGLEGIS |
| 51 | YISYDGSNNY NPSLKNRFTI SRDSAKNSFY LHMHSLRAED |
| | TAVYYCARDA |
| 101 | YSYGMDYWGQ GTTVTVSSAS TKGPSVFPLA PSSKSTSGGT |
| | AALGCLVKDY |
| 151 | FPEPVTVSWN SGALTSGVHT FPAVLQSSGL YSLSSVVTVP |
| | SSSLGTQTYI |
| 201 | CNVNHKPSNT KVDKKVEPKS CDKTHTCPPC PAPELLGGPS |
| | VFLFPPKPKD |
| 251 | TLMISRTPEV TCVVVDVSHE DPEVKFNWYV DGVEVHNAKT |
| | KPREEQYNST |
| 301 | YRVVSVLTVL HQDWLNGKEY KCKVSNKALP APIEKTISKA |
| | KGQPREPQVY |
| 351 | TLPPSRDELT KNQVSLTCLV KGFYPSDIAV EWESNGQPEN |
| | NYKTTPPVLD |
| 402 | SDGSFFLYSK LTVDKSRWQQ GNVFSCSVMH EALHNHYTQK |
| | SLSLSPG |

The solubility of the H1/L0 version of humanized 105 was found to be 9.9 mg/ml. Mutations were made to several light chain CDR residues thought to be responsible for self-association (and therefore insolubility) of the molecule. A version of the light chain having a mutation in CDRL2 of R at Kabat position 54 to K (version A), a second version having a mutation in CDRL2 of N at Kabat position 57 to S (version B), as well as a third version having both mutations in CDRL2 (comprising the K at Kabat position 54 and the S at Kabat position 57; version C) were made. Version A showed no precipitate at 28.6 mg/ml and version B showed no precipitate at 34.9 mg/ml.

Version A

(SEQ ID NO: 31)

| | |
|---|---|
| 1 | EIVLTQSPAT LSLSPGERAT LSCRASESVD NYGISFMHWY |
| | QQKPGQAPRL |
| 51 | LIYKASNLES GIPARFSGSG SGTDFTLTIS SLEPEDFAVY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

Version B

(SEQ ID NO: 32)

| | |
|---|---|
| 1 | EIVLTQSPAT LSLSPGERAT LSCRASESVD NYGISFMHWY |
| | QQKPGQAPRL |
| 51 | LIYRASSLES GIPARFSGSG SGTDFTLTIS SLEPEDFAVY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

Version C

(SEQ ID NO: 33)

| | |
|---|---|
| 1 | EIVLTQSPAT LSLSPGERAT LSCRASESVD NYGISFMHWY |
| | QQKPGQAPRL |
| 51 | LIYKASSLES GIPARFSGSG SGTDFTLTIS SLEPEDFAVY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

In an attempt to further improve solubility, a new version of the light chain was made which included both the R54K and N57S CDRL2 mutations found in version C of the light chain, and also included a new framework selected to provide an increased total charge, arriving at resulting sequence L10:

L10

(SEQ ID NO: 34)

| | |
|---|---|
| 1 | AIQLTQSPSS LSASVGDRVT ITCRASESVD NYGISFMHWY |
| | QQKPGKAPKL |
| 51 | LIYKASSLES GVPSRFSGSG SGTDFTLTIS SLQPEDFATY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

The L10 version of the light chain when combined with H1 showed a solubility of greater than 100 mg/ml.

Additional versions of the light chain were also made, including L12 and L13:

L12

(SEQ ID NO: 35)

| | |
|---|---|
| 1 | DIQLTQSPSS LSASVGDRVT ITCRASESVD NYGISFMHWY |
| | RQKPGKAPKL |
| 51 | LIYKASSLES GVPSRFSGRG SGTDFTLTIS SLQPEDFATY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

L13

(SEQ ID NO: 36)

| | |
|---|---|
| 1 | DIRLTQSPSS LSASVGQRVT ISCRASESVD NYGISFMHWY |
| | RQKPGKAPKL |
| 51 | LIYKASSLES GVPSRFSGRG SGTDFTLTIS SLQPEDFATY |
| | YCQQSNKDPY |
| 101 | TFGQGTKVEI KRTVAAPSVF IFPPSDEQLK SGTASVVCLL |
| | NNFYPREAKV |
| 151 | QWKVDNALQS GNSQESVTEQ DSKDSTYSLS STLTLSKADY |
| | EKHKVYACEV |
| 201 | THQGLSSPVT KSFNRGEC |

L12 in combination with H1 also showed no precipitate at 100 mg/ml, L13 in combination with H1 showed no precipitate at 48 mg/ml.

Additional heavy chain versions were also made, including H11 and H14.

H11

(SEQ ID NO: 37)

| | |
|---|---|
| 1 | EVQLVESGGG LVQPRGSLRL SCAVSGYSIT SGYYWNWIRQ |
| | APGKGLEWVS |
| 51 | YISYDGSNNY NPSLKNRFTI SRDNSKNTFY LQMNNLRAED |
| | TAAYYCARDA |
| 101 | YSYGMDYWGQ GTTVTVSSAS TKGPSVFPLA PSSKSTSGGT |
| | AALGCLVKDY |
| 151 | FPEPVTVSWN SGALTSGVHT FPAVLQSSGL YSLSSVVTVP |
| | SSSLGTQTYI |
| 201 | CNVNHKPSNT KVDKKVEPKS CDKTHTCPPC PAPELLGGPS |
| | VFLFPPKPKD |
| 251 | TLMISRTPEV TCVVVDVSHE DPEVKFNWYV DGVEVHNAKT |
| | KPREEQYNST |
| 301 | YRVVSVLTVL HQDWLNGKEY KCKVSNKALP APIEKTISKA |
| | KGQPREPQVY |
| 351 | TLPPSRDELT KNQVSLTCLV KGFYPSDIAV EWESNGQPEN |
| | NYKTTPPVLD |
| 401 | SDGSFFLYSK LTVDKSRWQQ GNVFSCSVMH EALHNHYTQK |
| | SLSLSPG |

H14

(SEQ ID NO: 38)

| | |
|---|---|
| 1 | EVQLQESGGG LVKPRGSLRL SCAVSGYSIT SGYYWNWIRQ |
| | APGKGLEWVS |
| 51 | YISYDGSNNY NPSLKNRFSI SRDNSKNTFY LKMNRLRAED |
| | SAAYYCARDA |
| 101 | YSYGMDYWGQ GTTVTVSSAS TKGPSVFPLA PSSKSTSGGT |
| | AALGCLVKDY |
| 151 | FPEPVTVSWN SGALTSGVHT FPAVLQSSGL YSLSSVVTVP |
| | SSSLGTQTYI |
| 201 | CNVNHKPSNT KVDKKVEPKS CDKTHTCPPC PAPELLGGPS |
| | VFLFPPKPKD |
| 251 | TLMISRTPEV TCVVVDVSHE DPEVKFNWYV DGVEVHNAKT |
| | KPREEQYNST |
| 301 | YRVVSVLTVL HQDWLNGKEY KCKVSNKALP APIEKTISKA |
| | KGQPREPQVY |
| 351 | TLPPSRDELT KNQVSLTCLV KGFYPSDIAV EWESNGQPEN |
| | NYKTTPPVLD |
| 401 | SDGSFFLYSK LTVDKSRWQQ GNVFSCSVMH EALHNHYTQK |
| | SLSLSPG |

Combinations of L10 with H11 or H14 showed much lower solubility than had been observed in combination with H1, 3.7 and greater than 28 mg/ml, respectively. Additional combinations were also tested and the data are presented in the table below:

| Heavy/Light chain combination | Solubility (mg/ml) |
|---|---|
| H1/L0 | 9.9 |
| H1/version A | >28.6 |
| H1/version B | >34.9 |
| H1/L10 | >100 |
| H1/L12 | >100 |
| H1/L13 | >48 |
| H11/L10 | 3.7 |
| H11/L12 | 11 |
| H11/L13 | 4.4 |
| H14/L10 | >28 |
| H14/L12 | >15 |

### Example 7

### Binding of Antibodies to LT

The availability of an LTbR binding site in the presence of a competitor was determined using the following methods:

Biacore Chip Preparation.

All experiments were performed using a Biacore 3000 instrument. The anti-Flag antibody M2 was immobilized on a CM5 sensorchip using the Biacore Amine Coupling kit according to manufacturer's instructions. Briefly, antibody was diluted to 50 pg/ml in 10 mM acetate, pH 5.0 and 30 μl was injected over chip surfaces that had been activated with a 30 μl injection of 1:1 N-hydroxsuccinimide (NHS): 1-Ethyl-3(3-dimethylaminopropyl)-carbodiimide hydrochloride (EDC). Excess free amine groups were then capped with a 50 μl injection of 1 M Ethanolamine. Typical immobilization level was 4000-6000 RU. All samples were prepared in assay buffer (10 mM HEPES pH 7.0+150 mM NaCl+0.05% detergent p-20+0.05% BSA). This same buffer was used as the running buffer during sample analysis. For immobilizations this same buffer without BSA was used.

Biacore Binding Assays.

Soluble Flag-tagged LTα1β2 was diluted in assay buffer to 200 nM and injected over the M2 derivatized surface, or an underivatized surface as a background control, at a flow rate of 25 μl/min. The surface was allowed to stabilize for 2 minutes while buffer flowed over the surface at 25 μl/min. A saturating concentration of competitor (i.e. 8 μM LTβR-Ig, 2 μM antibody LT105, 4 μM antibody B9, 4 μM antibody LT102 or 2 μM antibody 9B4; determined in separate experiments) was injected for 3 min at 25 μl/min. Again this surface was allowed to stabilize under buffer flow for 3 min. Following stabilization 20 μM monomeric LTβR in assay buffer was injected over the surface for 4 min at 25 μl/min. The surface was then regenerated with 2 injections of 3 M Guanidine hydrochloride in 0.5 M KCl.

The stoichiometry of binding of each component to the affinity captured LTα1β2 was determined as follows:

Competitor sites available=[(Competitor molecular weight)/(Ligand molecular weight)]×(Ligand response) (1)

Competitors bound=(net Competitor response)/(Competitor sites available) (2)

LTβR sites available=[(LTβR molecular weight)/(Ligand molecular weight)]×(net Ligand response) (3)

LTβR bound=(net LTβR response)/(LTβR sites available) (4)

Using these methods, 2LTβR binding sites were identified on LTα1β2, distinguished by their affinity for LTbR (site 1 exhibited an an affinity of approximately 50 nM and site 2 exhibited an affinity of approximately 1500 nM).

The antibodies tested bind with high apparent affinity (0.3 nM or better), while the Fab fragments tested (LT105 and B9) bind with low affinity (2 nM or weaker) as compared to the intact antibody. Thus, each of the antibodies tested binds to a single LTα1β2 trimer bivalently with high affinity.

As illustrated in the table below, in the presence of bound LTβR-Ig, LT105, LT102, or 9B4, there are no LTβR binding sites available, while in the presence of B9, one LTβR binding site remains available. Thus, while the prior art B9 antibody is capable of bivalent high affinity interaction with LTα1β2, it can block only one receptor binding site. In contrast, in the presence of bound LT105, LT102, and 9B4 antibodies (that have been demonstrated herein to more completely block the binding of LT to LTβR), no LTβR binding sites are available.

| | | molar equivalents LTβR |
|---|---|---|
| | molar equivalents | bound in presence of |
| Competitor | competitor bound | competitor |
| LTbR-Ig | 1.0 | 0 |
| LT105 | 1.0 | 0 |
| LT102 | 0.88 | 0.12 |
| 9B4 | 1.0 | 0 |
| B9 | 0.78 | 1.2 |
| no competitor | N/A | 1.5 |

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An isolated antibody that binds to lymphotoxin (LT) or antigen binding fragment thereof, wherein said antibody,
(a) blocks an LT-induced biological activity in a cell by at least about 70% under conditions in which a reference antibody, B9, (Produced by the cell line B9.C9.1, deposited with the ATCC under Accession number HB 11962) blocks the LT-induced biological activity in a cell by about 50%;
(b) blocks an LT-induced biological activity in a cell at an IC50 of less than 100 nM; or
(c) blocks LTβR-Ig binding to a cell by at least 85%.

2-3. (canceled)

4. The isolated antibody or molecule comprising an antigen binding region thereof of claim 1, wherein the LT-induced biological activity is IL-8 release.

5-6. (canceled)

7. The isolated antibody or molecule comprising an antigen binding region thereof of claim 1, wherein,
(a) the human constant region is an IgG1 constant region that has been altered to reduce binding to at least one Fc receptor or;
(b) the human constant region is an IgG1 constant region that has been altered to enhance binding to at least one Fc receptor.

8-15. (canceled)

16. The isolated antibody or antigen binding fragment thereof of claim 1, wherein the antibody or antigen binding fragment binds two sites on LT leaving no site for LTβR binding.

17-18. (canceled)

19. An isolated antibody, or antigen binding fragment thereof, that specifically binds to an epitope of LT, wherein the binding to the LT epitope by the antibody is competitively blocked in a dose-dependent manner by,
(a) the 102 antibody;
(b) the AOD9 antibody;
(c) 101/103 antibody;
(d) the 105 antibody;
(e) the 9B4 antibody;
(f) the A1D5 antibody;
(g) the 107 antibody; or
(h) the 108 antibody.

20. The isolated antibody, or antigen binding fragment thereof, of claim 19, wherein
(a) amino acids 193 and 194 of LTβ are critical for binding of the 102 antibody;
(b) amino acids 96, 97, 98, 106, 107, and 108 of LTβ are critical for binding of the 105 antibody;
(c) amino acids 96, 97, and 98 of LTβ are critical for binding of the 9B4 antibody;
(d) amino acid 172 of LTβ is critical for binding of the A1D5 antibody; and
(e) amino acids 151 and 153 of LTβ are critical for binding of the 107 antibody.

21. An isolated antibody, or antigen binding fragment thereof, that specifically binds to the same epitope as the antibody or fragment of claim 1, or competes for binding to LT with the antibody or fragment of claim 1.

22. The isolated antibody or fragment of claim 21, that specifically binds to an epitope of LT, wherein the binding to the LT epitope by the antibody is competitively blocked in a dose-dependent manner by,
(a) the 102 antibody;
(b) the AOD9 antibody;
(c) 101/103 antibody;
(d) the 105 antibody;
(e) the 9B4 antibody;
(f) the A1D5 antibody;
(g) the 107 antibody; or
(h) the 108 antibody.

23-34. (canceled)

35. A lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein the light and heavy chain CDRs are derived from an antibody selected from the group consisting of AOD9, 108, 107, A1D5, 102,101/103, 9B4 and 105.

36-43. (canceled)

44. A lymphotoxin binding molecule comprising a heavy chain variable region comprising heavy chain CDRs CDRH1, CDRH2 and CDRH3 and light chain variable region comprising light chain CDRs CDRL1, CDRL2, and CDRL3, wherein
(a) CDRH1 comprises the sequence GFSLX1X2Y/SGX3H;
(b) CDRH2 comprises the sequence VIWX1GGX2TX3X4NAX5FX6S;
(c) CDRL1 comprises the sequence RASX1SVX2X3X4X5 or X1ASQDX2X3X4X5LX6;
(d) CDRL2 comprises the sequence RAX1RLX2D;
(e) CDRL2 comprises the sequence X1X2SX3X4X5S;
(f) CDRL3 comprises the sequence X1QX2X3X4X5PX6T; or
(g) CDRL3 comprises the sequence LX1X2DX4FPX6T;
and wherein X is any amino acid.

45-50. (canceled)

51. A lymphotoxin binding molecule comprising a light chain variable region comprising heavy chain CDRs CDRH1, CDRH2 and CDRH3 of a 105 antibody variant and light chain variable region comprising light chain CDRs CDRL1, CDRL2, and CDRL3 of a 105 variant.

52-54. (canceled)

55. The lymphotoxin binding molecule of claim 51, wherein the binding molecule comprises the light chain variable region of the 105 variant version L10 and the heavy chain variable region of the 105 variant version H1.

56. A composition comprising the isolated antibody or antigen binding region thereof of claim 1, and a carrier.

57. (canceled)

58. A method of treating a subject that would benefit from treatment with an anti-LT binding molecule comprising administering the antibody of claim 1 and a pharmaceutically acceptable carrier to the subject such that treatment occurs.

59. (canceled)

60. The method of claim 58, wherein the inflammatory disorder is selected from group consisting of rheumatoid arthritis, multiple sclerosis, Crohn's disease, ulcerative colitis, a transplant, lupus, inflammatory liver disease, psoriasis, Sjorgren's syndrome, multiple sclerosis (e.g., SPMS), viral-induced hepatitis, autoimmune hepatitis, type I diabetes, atherosclerosis, and viral shock syndrome.

61-62. (canceled)

63. The method of claim 58, wherein the cancer is selected from the group consisting of multiple myeloma and indolent follicular lymphoma.

64. A nucleic acid molecule encoding the antibody of claim 1.

65. A nucleic acid molecule encoding the binding molecule of claim 44.

66. The nucleic acid molecule of claim 64 which is in a vector.

67. A host cell comprising the vector of claim 66.

68. A method of producing the antibody or binding molecule, comprising
(i) culturing the host cell of claim 67 such that the antibody or binding molecule is secreted in host cell culture media (ii) isolating the antibody or binding molecule from the media.

69. Use of a composition of claim 56 in the manufacture of a medicament for the treatment of a disorder associated with inflammation.

70. (canceled)
